(19) United States
(12) Patent Application Publication
(10) Pub. No.: US 2023/0332107 A1
(43) Pub. Date: Oct. 19, 2023

(54) CELL THERAPY FOR DIABETES

(71) Applicant: Vertex Pharmaceuticals Incorporated, Boston, MA (US)

(72) Inventors: Felicia PAGLIUCA, Boston, MA (US); George HARB, Boston, MA (US); Jason GAGLIA, Cambridge, MA (US); Andrea VERGANI, Boston, MA (US); Christopher THANOS, Boston, MA (US)

(21) Appl. No.: 18/162,129

(22) Filed: Jan. 31, 2023

Related U.S. Application Data
(60) Provisional application No. 63/305,575, filed on Feb. 1, 2022.

(51) Int. Cl. C12N 5/071

(52) U.S. Cl. CPC C12N 5/0678; C12N 2501/15; C12N 2501/727; C12N 2501/999; C12N 2506/02

(57) ABSTRACT

Disclosed herein are compositions, kits, and methods related to cell therapy for a disease characterized by high blood glucose levels over a long period of time, such as diabetes. In some aspects, the methods provided herein relate to administration of non-native pancreatic cells to subject with a disease characterized by high blood glucose levels over a long period of time, such as diabetes. In some aspects, the disclosure provides pharmaceutical compositions including non-native cells.

### CROSS REFERENCE TO RLEATED APPLICATIONS

This application claims the benefit of U.S. Provisional Pat. Application No. 63/305,575, filed Feb. 1, 2022, which application is herein specifically incorporated by reference in its entirety.

### INCORPORATION BY REFERENCE OF SEQUENCE LISTING

The Sequence Listing in an XML filed, named as 41639_SequenceListing.xml of 28 KB, created on Jan. 19, 2023, and submitted via Patent Center, is incorporated herein by reference.

### BACKGROUND

Generation of stem cell derived β-cells can provide a potentially useful step toward the generation of islets and pancreatic organs. One of the rapidly growing diseases that may be treatable by stem cell derived tissues is diabetes. Type 1 diabetes results from autoimmune destruction of β-cells in the pancreatic islet. Type 2 diabetes results from peripheral tissue insulin resistance and β-cell dysfunction. Diabetic patients, particularly those suffering from type 1 diabetes, can potentially be cured through transplantation of new β-cells. Patients transplanted with cadaveric human islets can be made insulin independent for 5 years or longer via this strategy, but this approach is limited because of the scarcity and quality of donor islets. Generation of an unlimited supply of human β-cells from stem cells can extend this therapy to millions of new patients and can be an important test case for translating stem cell biology into the clinic.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY

Disclosed herein, in some aspects, is a method of treating a subject in need thereof, comprising: administering to the subject via infusion a first pharmaceutical composition comprising a population of cells in a liquid suspension, wherein the population of cells comprises from 1 × 10⁸ to 10 × 10⁸ (e.g., 3 × 10⁸ to 8.5 × 10⁸) cells, and wherein the population of cells comprises non-native cells expressing C-peptide and ISL1. In some cases, the method elevates stimulated blood C-peptide level of the subject to at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, 550 pmol/L, 700 pmol/L, 800 pmol/L, 900 pmol/L, 1000 pmol/L, 1400 pmol/L, or between 150-350 pmol/L, 250-400 pmol/L, 400-1400 pmol/L, 400-1200 pmol/L, 400-1000 pmol/L, 400-800 pmol/L, 400-600 pmol/L, 600-800 pmol/L, 600-1400 pmol/L, or 900-1200 pmol/L, when measured using a mixed meal tolerance test at least about 1, 2, 3, 4, 5, or 6 months after the administration. In some cases, the method elevates stimulated blood C-peptide level of the subject to at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, or 550 pmol/L, 700 pmol/L, 800 pmol/L, 900 pmol/L, 1000 pmol/L, 1400 pmol/L or between 150-350 pmol/L, 250-400 pmol/L, 400-1400 pmol/L, 400-1200 pmol/L, 400-1000 pmol/L, 400-800 pmol/L, 400-600 pmol/L, 600-800 pmol/L, 600-1400 pmol/L, or 900-1200 pmol/L, when measured using a mixed meal tolerance test about 1, 2, 3, 4, 5, 6, 7, 8, 9, 10, 11, or 12 months after the administration. In some cases, the method elevates stimulated blood C-peptide level of the subject to at least at least 200 pmol/L, 300 pmol/L, 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, 550 pmol/L, 600 pmol/L, or 650 pmol/L, or between 150-350 pmol/L, 200-700 pmol/L, 150-700 pmol/L, 300-650 pmol/L, or 500-700 pmol/L, when measured using a mixed meal tolerance test about 3 months after the administration.

In some cases, the method elevates stimulated blood C-peptide level of the subject to about at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, 550 pmol/L, 560 pmol/L, 700 pmol/L, 800 pmol/L, 900 pmol/L, 1000 pmol/L, 1400 pmol/L or between 150-350 pmol/L, 250-400 pmol/L, 400-1400 pmol/L, 400-1200 pmol/L, 400-1000 pmol/L, 400-800 pmol/L, 400-600 pmol/L, 600-800 pmol/L, 600-1400 pmol/L, or 900-1200 pmol/L, when measured using a mixed meal tolerance test about 6 months after the administration. In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured at least about 1, 2, 3, 4, 5, or 6 months after the administration. In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured about 1, 2, 3, 4, 5, 6, 7, 8, 9, 10, 11, or 12 months after the administration. In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured about 3 months after the administration. In some cases, the method elevates blood C-peptide level of the subject under fasting condition to about 200 pmol/L, 220 pmol/L, 240 pmol/L, 260 pmol/L, or 280 pmol/L, when measured about 3 months after the administration.

In some cases, prior to the administration, the subject has stimulated blood C-peptide level of less than 100 pmol/L, 80 pmol/L, 60 pmol/L, 40 pmol/L, 30 pmol/L, 20 pmol/L, or 10 pmol/L when measured using a mixed meal tolerance test. In some cases, prior to the administration, the subject has undectable stimulated blood C-peptide level when measured using a mixed meal tolerance test. In some cases, prior to the administration, the subject has undetectable stimulated blood C-peptide level when measured under fasting condition.

In some cases, the method reduces hemoglobin A1c level (Hb1Ac) level of the subject to less than 8%, 7.5%, 7.0%, 6.5%, 6.0%, 5.5% or between 5-8%, 5-7.5%, 5-6.5%, 5-5.5%, 5.5-8%, 5.5-7%, 5.5-6.5%, 6-8%, 6-7%, or 6-6.5%, when measured using a HbA1c test at least about 1, 2, 3, 4, 5, or 6 months after the administration. In some cases, the method reduces Hb1Ac level of the subject to less than 8%, 7.5%, 7.0%, 6.5%, 6.0%, 5.5% or 5% or between 5-8%, 5-7.5%, 5-6.5%, 5-5.5%, 5.5-8%, 5.5-7%, 5.5-6.5%, 6-8%, 6-7%, or 6-6.5%, when measured using a HbA1c test about 1, 2, 3, 4, 5, 6, 7, 8, 9, 10, 11, or 12 months after the administration. In some cases, the method reduces Hb1Ac level of the subject to less than 8%, 7.5%, 7.0%, or 6.75% or between 5-8.0%, 6.5-8.0%, 6.5-7.5%, 6.5-7.0%, or 7-8% when measured about 3 months after the administration. In some cases, the method reduces Hb1Ac level of the subject to less than 8%, 7.5%, 7.0%, 6.5%, 6.0%, 5.5%, or 5%, or between 4.5-8%, 5-8%, 5-7.5%, 5-7%, 5-6.5%, 5-5.5%, 5.5-8%, 5.5-7%, 5.5-6.5%, 6-8%, 6-7%, or 6-6.5%when measured about 9 months after the administration.

In some cases, prior to the administration, the subject has a baseline Hb1Ac level of more than 7.0%, 7.3%, 7.8%, 8.0%, 8.1%, 8.2%, 8.3%, 8.4%, 8.5%, 8.6%, 8.8%, or 9.0%. In some cases, prior to the administration, the subject receives infusion of insulin at a level of at least about 20 U/day, 22 U/day, 24 U/day, 26 U/day, 28 U/day, 30 U/day, 32 U/day, or 34 U/day. In some cases, prior to the administration, the subject has a medical history of severe hypoglycemic events. In some cases, prior to the administration, the subject has a medical history of impaired hypoglycemia awareness. In some cases, prior to the administration, the subject presented with no residual endogenous islet cell function. In some cases, the no residual endogenous islet cell function is indicated by undetectable fasting C-peptide and by undetectable stimulated C-peptide at the Mixed Meal Tolerance Test. In some cases, the no residual endogenous islet cell function is indicated by sustained stimulated glucose levels greater than 350, 400, 450, 475, or 500 mg/dL at the Mixed Meal Tolerance Test. In some cases, prior to the administration, the subject had an HbA1c level greater than 7.7%, 8%, 8.2%, or 8.5%. In some cases, prior to the administration, the subject was receiving greater than 10, 15, 20, 25, 30 or 35 units per day of insulin.

In some cases, the subject has a disease characterized by high blood sugar levels over a prolonged period of time. In some cases, the disease is diabetes. In some cases, the disease is Type 1 diabetes.

In some cases, the population of cells comprises at most about 12 × 10⁸, 10 × 10⁸, 8 × 10⁸, 7 × 10⁸, 6.5 × 10⁸, 6 × 10⁸, 5.5 × 10⁸, 5 × 10⁸, 4.5 × 10⁸, 4 × 10⁸, 3.5 × 10⁸, 3 × 10⁸, 2.5 × 10⁸, or 2 × 10⁸ cells. In some cases, the population of cells comprises about 1 × 10⁸ to about 7 × 10⁸, about 1 × 10⁸ to about 6 × 10⁸, about 1 × 10⁸ to about 5 × 10⁸, about 1 × 10⁸ to about 4 × 10⁸, about 1 × 10⁸ to about 3 × 10⁸, about 1 × 10⁸ to about 2 × 10⁸, about 1.5 × 10⁸ to about 6.5 × 10⁸, about 2 × 10⁸ to about 7 × 10⁸, about 2 × 10⁸ to about 6 × 10⁸, about 2 × 10⁸ to about 5 × 10⁸, about 2 × 10⁸ to about 4 × 10⁸, about 2 × 10⁸ to about 3 × 10⁸, about 2.5 × 10⁸ to about 5.5 × 10⁸, about 3 × 10⁸ to about 7 × 10⁸, about 3 × 10⁸ to about 6 × 10⁸, about 3 × 10⁸ to about 5 × 10⁸, about 3 × 10⁸ to about 4 × 10⁸, about 3.5 × 10⁸ to about 4.5 × 10⁸, or about 3.8 × 10⁸ to about 42 × 10⁸ cells. In some cases, the population of cells comprises about about 3.5 × 10⁸ to about 45 × 10⁸ cells. In some cases, the population of cells comprises about about 3.5 × 10⁸ to about 8.5 × 10⁸ cells.

In some cases,the subject is administered via infusion a second pharmaceutical composition comprising a population of cells in a liquid suspension, wherein the population of cells comprises from 1 × 10⁸ to 10 × 10⁸ (e.g., from 3 × 10⁸ to 8.5 × 10⁸, from 4 × 10⁸ to 8.5 × 10⁸, or from 5 × 10⁸ to 8.5 × 10⁸ cells), and wherein the population of cells comprises non-native cells expressing C-peptide and ISL1, wherein the second pharmaceutical composition is administered to the subject at a later point in time than the first pharmaceutical composition. In some cases, the second pharmaceutical composition is administered to the subject at least 3 months, 4 months, 5 months, 6 months, 7 months, 8 months, 9 months, 10 months, 11 months, 12 months, 15 months or between 3-12 months, 3-10 months, 3-9 months, 3-7 months, 3-5 months, 5-12 months, 8-10 months, 7-12 months, 9-15 months, or 9-12 months after the subject is administered the first pharmaceutical composition. In some cases, the first pharmaceutical composition comprises 3.5 × 10⁸ to about 8.5 × 10⁸ cells and wherein the second pharmaceutical composition comprises 3.5 × 10⁸ to about 8.5 × 10⁸ cells. In some cases, the first pharmaceutical composition comprises 3.5 × 10⁸ to about 8.5 × 10⁸ cells and wherein the second pharmaceutical composition comprises 3.5 × 10⁸ to about 4.5 × 10⁸ cells.

In some cases, the subject is also administered at least one immunosuppressant. In some cases, the at least one immunosuppressant comprises selected from the group consisting of Thymoglobulin, Etanercept, Basiliximab, Tacrolimus, Sirolimus, and Mycophenolate mofetil. In some cases, the subject is also administered Thymoglobulin and Etanercept. In some cases, the subject is also administered Thymoglobulin and Basiliximab. In some cases, the subject is also administered Tacrolimus and Sirolimus. In some cases, the subject is also administered Tacrolimus and Basiliximab. In some cases, the subject is also administered is also administered Sirolimus and Mycophenolate..

In some cases, the first pharmaceutical composition comprises a sugar. In some cases, the sugar is sucrose or glucose. In some cases, the liquid suspension comprises the sugar at a concentration of between about 0.05% and about 1.5%.

In some cases, the first pharmaceutical composition comprises a CMRL medium. In some cases, the first pharmaceutical composition comprises HypoThermosol® FRS Preservation Media.

In some cases, the population of cells in the first pharmaceutical composition further comprises non-native cells expressing glucagon but not somatostatin, and non-native cells expressing somatostatin but not glucagon.

In some cases of the method, (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells in the first pharmaceutical composition express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells in the first pharmaceutical composition express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells in the first pharmaceutical composition express somatostatin but not glucagon.

In some cases of the method, (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases of the method, (a) 35-60% of the cells in the population of cells express C-peptide and ISL1 but not VMAT1; (b) 4-25%, of the cells in the population of cells express glucagon but not somatostatin; and (c) 1-10% of the cells in the population of cells express somatostatin but not glucagon. In some cases of the method, (a) 40-60% of the cells in the population of cells express C-peptide and ISL1 but not VMAT1; (b) 10-25%, of the cells in the population of cells express glucagon but not somatostatin; and (c) 4-10% of the cells in the population of cells express somatostatin but not glucagon.

In some cases, less than 40%, less than 35%, less than 30%, less than 25%, less than 20%, less than 18%, less than 15%, less than 12%, or less than 10% of the cells in the population of cells express VMAT1 but not C-peptide. In some cases, no less than 50%, 40%, 30%, or 20% of the cells in the first pharmaceutical composition are NKX6.1⁺/ISL1⁺ cells, as determined by flow cytometry. In some cases, no less than 20% of the cells in the first pharmaceutical composition are NKX6.1⁺/ISL1⁺ cells, as determined by flow cytometry. In some cases, no less than 40%, 35%, 30%, 26 %, 25%, or 20% of the cells in the first pharmaceutical composition are NKX6.1⁻/ISL1⁺ cells, as determined by flow cytometry. In some cases, no less than 26 % of the cells in the first pharmaceutical composition are NKX6.1⁻/ISL1⁺ cells, as determined by flow cytometry. In some cases, between 5-25%, 5-40%, 5-35%, or 8-20% of the cells in the first pharmaceutical composition are NKX6.1⁻/ISL1⁺ cells, as determined by flow cytometry. In some cases, no more than 50%, 45%, 40%, 35%, 30%, or 25% of the cells in the first pharmaceutical composition are NKX6.1⁺/ISL1⁻ cells, as determined by flow cytometry. In some cases, no more than 50% of the cells in the first pharmaceutical composition are NKX6.1⁺/ISL1⁻ cells, as determined by flow cytometry. In some cases, between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, 40-50% of the cells in the pharmaceutical composition are NKX6.1⁺/ISL1⁺ cells, as determined by flow cytometry. In some cases, between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, or 40-50% of the cells in the pharmaceutical composition are NKX6.1⁻/ISL1⁺ cells, as determined by flow cytometry. In some cases, between 20-50%, 20-45%, 20-40%, 20-35%, 20-30%, 20-25%, 25-50%, 25-40%, 25-35%, 30-60%, 30-50%, 30-40%, 30-35%, 35-50%, 40-50%, 10-20%, or 10-25% of the cells in the pharmaceutical composition are NKX6.1⁺/ISL1⁻ cells, as determined by flow cytometry. In some cases of the method, at least 20% of the cells in the composition are NKX6.1⁺/ISL1⁺ cells; at least 25% of the cells in the composition are NKX6.1⁻/ISL1⁺ cells; and 10-50% of the cells in the composition are NKX6.1⁺/ISL1⁻ cells. In some cases, the composition comprises NKX6.1⁺/ISL1⁺ cells that display a GSIS in vitro. In some cases, the composition comprises NKX6.1⁺/ISL1⁺ cells that display a GSIS in vivo. In some cases, the population of cells are generated from stem cells in vitro. In some cases, non-native cells expressing C-peptide and ISL1 but not VMAT1 in the population of cells exhibit glucose-stimulated insulin secretion response in vitro. In some cases, secretion of insulin by the non-native cells expressing C-peptide and ISL1 but not VMAT1 in response to a glucose challenge is proportional to glucose concentration of the glucose challenge. In some cases, secretion of insulin by the non-native cells expressing C-peptide and ISL1 but not VMAT1 secrete insulin in response to a first glucose challenge, a second glucose challenge, and a third glucose challenge, wherein the first glucose challenge, the second glucose challenge, and the third glucose challenge are applied sequentially.

In some cases, at least a portion of the cells in the population of cells are present in plurality of cell clusters. In some cases, the cell clusters are about 50 µm to about 500 µm, about 50 µm to about 400 µm, about 50 µm to about 300 µm, about 60 µm to about 400 µm, about 60 µm to about 300 µm, about 60 µm to about 250 µm, about 75 µm to about 400 µm, about 75 µm to about 300 µm, about 75 µm to about 250 µm, about 125 µm to about 225 µm, about 130 µm to about 160 µm, about 170 µm to about 225 µm, about 140 µm to about 200 µm, about 140 µm to about 170 µm, about 160 µm to about 220 µm, about 170 µm to about 215 µm, or about 170 µm to about 200 µm in diameter. In some embodiments, a cell cluster is between about 80 and 270 microns in diameter.

In some cases, the population of cells are present as a single cell suspension in the first pharmaceutical composition.

In some cases, in the first pharmaceutical composition, the population of cells is suspended in a serum-free solution.

In some cases, the method further comprises administering to the subject an immune response modulator. In some cases, the immune response modulator is administered concurrently with the first pharmaceutical composition. In some cases, the immune response modulator is administered prior to or subsequent to the first pharmaceutical composition. In some cases, the immune response modulator is not a steroid. In some cases, the immune response modulator comprises azathioprine, mycophenolic acid, leflunomide, teriflunomide, methotrexate, tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, anti-lymphocyte globulin antibodies, CTLA-4, abatacept, belatacept, etanercept, pegsunercept, aflibercept, alefacept, rilonacept, eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, atorolimumab, cedelizumab, fontolizumab, maslimomab, morolimumab, pexelizumab, reslizumab, rovelizumab, siplizumab, talizumab, telimomab aritox, vapaliximab, vepalimomab, alemtuzumab, mycophenolate mofetil, FTY720, or any combination thereof. In some cases, the immune response modulator comprises tacrolimus, cyclosporine, mycophenolate mofetil, azathioprine, everolimus, sirolimus, abatacept, belatacept, antithymocyte globulin, alemtuzumab, rituximab, basiliximab, daclizumab, muromonab-CD3, efalizumab, FTY720, or any combination thereof. In some cases, the immune response modulator comprises a corticosteroid.

In some cases, the first pharmaceutical composition is infused into portal vein of the subject.

In some cases, the subject receives a single dose of the first pharmaceutical composition.

In some cases, the non-native cells expressing C-peptide and ISL1 cells were obtained from a culture of cells comprising PDX1-positive, NKX6.1-positive, insulin-positive cells treated with i) a TGF-β signaling pathway inhibitor (e.g., Alk5i), ii) a thyroid hormone signaling pathway activator (e.g., GC-1), iii) a BMP pathway inhibitor (e.g., LDN193189), iv) a ROCK inhibitor (e.g., thiazovivin), v) a protein kinase inhibitor (e.g., staurosporine), and/or vi) an epigenetic modifier (e.g., DZNEP). In some cases, the non-native cells expressing C-peptide and ISL1 cells were obtained from a culture of cells comprising PDX1-positive, NKX6.1-positive, insulin-positive cells treated with i) a TGF-β signaling pathway inhibitor (e.g., Alk5i), ii) a thyroid hormone signaling pathway activator (e.g., GC-1), iii) a BMP pathway inhibitor (e.g., LDN193189), iv) a ROCK inhibitor (e.g., thiazovivin), v) a protein kinase inhibitor (e.g., staurosporine), and/or vi) an epigenetic modifier (e.g., DZNEP) for at least 3 days. In some cases, the PDX1-positive, NKX6.1-positive, insulin-positive cells were also treated with human serum albumin. In some cases, the PDX1-positive, NKX6.1-positive, insulin-positive cells were also treated with human serum albumin for at least 6 days. In some cases,the PDX1-positive, NKX6.1-positive, insulin-positive cells were obtained from a culture of cells comprising PDX1-positive, NKX6.1-positive, insulin-negative cells treated with i) a TGF-β signaling pathway inhibitor (e.g., Alk5i), ii) a TH signaling pathway activator (e.g., GC-1), iii) at least one SHH pathway inhibitor (e.g., Sant1), iv) a RA signaling pathway activator (e.g., retinoic acid), v) a γ-secretase inhibitor (e.g., XXI), vi) at least one growth factor from the epidermal growth factor (EGF) family (e.g., betacellulin), vii) a BMP pathway inhibitor (e.g., LDN193189), viii) a ROCK inhibitor (e.g., thiazovivin), ix) a protein kinase inhibitor (e.g., staurosporine), and/or x) an epigenetic modifier (e.g., DZNEP).

In some cases, the PDX1-positive, NKX6.1-positive, insulin-positive cells were obtained from a culture of cells comprising PDX1-positive, NKX6.1-positive, insulin-negative cells treated with i) a TGF-β signaling pathway inhibitor (e.g., Alk5i), ii) a TH signaling pathway activator (e.g., GC-1), iii) at least one SHH pathway inhibitor (e.g., Sant1), iv) a RA signaling pathway activator (e.g., retinoic acid), v) a γ-secretase inhibitor (e.g., XXI), vi) at least one growth factor from the epidermal growth factor (EGF) family (e.g., betacellulin), vii) a BMP pathway inhibitor (e.g., LDN193189), viii) a ROCK inhibitor (e.g., thiazovivin), ix) a protein kinase inhibitor (e.g., staurosporine), and/or x) an epigenetic modifier (e.g., DZNEP) for 2 or 3 days, followed by contacting the PDX1-positive, NKX6.1-positive pancreatic progenitor cells with i) a TGF-β signaling pathway inhibitor (e.g., Alk5i), ii) a TH signaling pathway activator (e.g., GC-1), iii) a γ-secretase inhibitor (e.g., XXI), iv) a BMP pathway inhibitor (e.g., LDN193189), v) a ROCK inhibitor (e.g., thiazovivin), vi) a protein kinase inhibitor (e.g., staurosporine), and vii) an epigenetic modifier (e.g., DZNEP), for a period of four or five days.

In some cases, the PDX1-positive, NKX6.1-positive, insulin-positive cells were obtained from a culture of cells comprising PDX1-positive, NKX6.1-positive, insulin-negative cells treated with i) a TGF-β signaling pathway inhibitor (e.g., Alk5i), ii) a TH signaling pathway activator (e.g., GC-1), iii) at least one SHH pathway inhibitor (e.g., Sant1), iv) a RA signaling pathway activator (e.g., retinoic acid), v) a γ-secretase inhibitor (e.g., XXI), vi) at least one growth factor from the epidermal growth factor (EGF) family (e.g., betacellulin), vii) a BMP pathway inhibitor (e.g., LDN193189), viii) a ROCK inhibitor (e.g., thiazovivin), ix) a protein kinase inhibitor (e.g., staurosporine), and/or x) an epigenetic modifier (e.g., DZNEP) for 2 or 3 days, followed by contacting the PDX1-positive, NKX6.1-positive pancreatic progenitor cells with i) a TGF-β signaling pathway inhibitor (e.g., Alk5i), ii) a TH signaling pathway activator (e.g., GC-1), iii) a γ-secretase inhibitor (e.g., XXI), iv) a BMP pathway inhibitor (e.g., LDN193189), v) a ROCK inhibitor (e.g., thiazovivin), vi) a protein kinase inhibitor (e.g., staurosporine), and vii) an epigenetic modifier (e.g., DZNEP), for a period of four or five days in the absence of i) a SHH pathway inhibitor (e.g., Sant1), ii) a RA signaling pathway activator (e.g., retinoic acid), and iii) a growth factor from the epidermal growth factor (EGF) family (e.g., betacellulin).

In some cases,the PDX1-positive, NKX6.1-positive, insulin-negative cells were obtained from a culture of cells comprising PDX1-positive, NKX6.1-negative cells treated with i) at least one growth factor from the FGF family (e.g., KGF), ii) at least one SHH pathway inhibitor (e.g., Sant1), iii) a RA signaling pathway activator (e.g., retinoic acid), iv) a ROCK inhibitor (e.g., thiazovivin) and v) at least one factor from TGFβ superfamily (e.g., activin A).

In some cases, the PDX1-positive, NKX6.1-positive, insulin-negative cells were obtained from a culture of cells comprising PDX1-positive, NKX6.1-negative cells treated with i) at least one growth factor from the FGF family (e.g., KGF), ii) at least one SHH pathway inhibitor (e.g., Sant1), iii) a RA signaling pathway activator (e.g., retinoic acid), iv) a ROCK inhibitor (e.g., thiazovivin) and v) at least one factor from TGFβ superfamily (e.g., activin A), for a period of 5 days.

In some cases, the PDX1-positive, NKX6.1-negative cells were obtained from a culture of cells comprising PDX1-negative primitive gut cells treated with i) retinoic acid signaling pathway activator, ii) at least one factor from the FGF family (e.g., KGF), iii) a SHH pathway inhibitor (e.g., Sant1), iv) a PKC activator (e.g., PDBU), v) a ROCK inhibitor (e.g., thiazovivin), and a vi) BMP pathway inhibitor (e.g., DMH-1).

In some cases, the PDX1-positive, NKX6.1-negative cells were obtained from a culture of cells comprising PDX1-negative primitive gut cells treated with i) retinoic acid signaling pathway activator, ii) at least one factor from the FGF family (e.g., KGF), iii) a SHH pathway inhibitor (e.g., Sant1), iv) a PKC activator (e.g., PDBU), v) a ROCK inhibitor (e.g., thiazovivin), and a vi) BMP pathway inhibitor (e.g., DMH-1) for 1 day, followed by contacting the cells with i) retinoic acid signaling pathway activator, ii) at least one factor from the FGF (e.g., KGF) family, iii) a SHH pathway inhibitor (e.g., Sant1), iv) a PKC activator (e.g., PDBU), v) a ROCK inhibitor (e.g., thiazovivin) for another day.

In some cases, the PDX1-positive, NKX6.1-negative cells were obtained from a culture of cells comprising PDX1-negative primitive gut cells treated with i) retinoic acid signaling pathway activator, ii) at least one factor from the FGF family (e.g., KGF), iii) a SHH pathway inhibitor (e.g., Sant1), iv) a PKC activator (e.g., PDBU), v) a ROCK inhibitor (e.g., thiazovivin), and a vi) BMP pathway inhibitor (e.g., DMH-1) for 1 day, followed by contacting the cells with i) retinoic acid signaling pathway activator, ii) at least one factor from the FGF (e.g., KGF) family, iii) a SHH pathway inhibitor (e.g., Sant1), iv) a PKC activator (e.g., PDBU), v) a ROCK inhibitor (e.g., thiazovivin) for another day in the absence of the BMP pathway inhibitor.

In some cases, the PDX1-negative primitive gut cells were obtained from a culture comprising definitive endoderm cells treated with a factor from the FGF family (e.g., KGF).

In some cases, the PDX1-negative primitive gut cells were obtained from a culture comprising definitive endoderm cells treated with a factor from the FGF family (e.g., KGF) for a period of 3 days.

In some cases, the definitive endoderm cells were obtained from a culture comprising pluripotent stem cells treated with a factor from TGFβ superfamily (e.g., activin A) and a WNT signaling pathway activator (e.g., CHIR99021). In some cases, the definitive endoderm cells were obtained from a culture comprising pluripotent stem cells treated with a factor from TGFβ superfamily (e.g., activin A) and a WNT signaling pathway activator (e.g., CHIR99021) for one day followed by contacting the cells with a factor from the TGFβ superfamily (e.g., activin A) for two additional days.

Disclosed herein, in some aspects, is a pharmaceutical composition formulated for infusion, comprising a population of cells in a liquid suspension, wherein the population of cells comprises from about 1 × 10⁸ to about 10 × 10⁸ (e.g., 3 × 10⁸ to 8.5 × 10⁸) cells, and wherein the population of cells comprises non-native cells expressing C-peptide and ISL1.

In some cases, the population of cells comprises at most about 12 × 10⁸, 10 × 10⁸, 8 × 10⁸, 7 × 10⁸, 6.5 × 10⁸, 6 × 10⁸, 5.5 × 10⁸, 5 × 10⁸, 4.5 × 10⁸, 4 × 10⁸, 3.5 × 10⁸, 3 × 10⁸, 2.5 × 10⁸, or 2 × 10⁸ cells. In some cases, the population of cells comprises about 1 × 10⁸ to about 7 × 10⁸, about 1 × 10⁸ to about 6 × 10⁸, about 1 × 10⁸ to about 5 × 10⁸, about 1 × 10⁸ to about 4 × 10⁸, about 1 × 10⁸ to about 3 × 10⁸, about 1 × 10⁸ to about 2 × 10⁸, about 1.5 × 10⁸ to about 6.5 × 10⁸, about 2 × 10⁸ to about 7 × 10⁸, about 2 × 10⁸ to about 6 × 10⁸, about 2 × 10⁸ to about 5 × 10⁸, about 2 × 10⁸ to about 4 × 10⁸, about 2 × 10⁸ to about 3 × 10⁸, about 2.5 × 10⁸ to about 5.5 × 10⁸, about 3 × 10⁸ to about 7 × 10⁸, about 3 × 10⁸ to about 6 × 10⁸, about 3 × 10⁸ to about 5 × 10⁸, about 3 × 10⁸ to about 4 × 10⁸, about 3.5 × 10⁸ to about 4.5 × 10⁸, or about 3.8 × 10⁸ to about 4.2 X 10⁸ cells. In some cases, the population of cells comprises about about 3.5 × 10⁸ to about 4.5 × 10⁸ cells. In some cases, the population of cells comprises about about 3.5 × 10⁸ to about 8.5 × 10⁸ cells. In some cases, the population of cells further comprises non-native cells expressing glucagon but not somatostatin, and non-native cells expressing somatostatin but not glucagon.

In some cases of the pharmaceutical composition, (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases of the pharmaceutical composition, (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases of the pharmaceutical composition, (a) 35-60% of the cells in the population of cells express C-peptide and ISL1 but not VMAT1; (b) 4-25%, of the cells in the population of cells express glucagon but not somatostatin; and (c) 1-10% of the cells in the population of cells express somatostatin but not glucagon.

In some cases of the pharmaceutical composition, (a) 40-60% of the cells in the population of cells express C-peptide and ISL1 but not VMAT1; (b) 10-25%, of the cells in the population of cells express glucagon but not somatostatin; and (c) 4-10% of the cells in the population of cells express somatostatin but not glucagon.

In some cases, less than 40%, less than 35%, less than 30%, less than 25%, less than 20%, less than 18%, less than 15%, less than 12%, or less than 10% of the cells in the population of cells express VMAT1 but not C-peptide. In some cases, no less than 50%, 40%, 30%, or 20% of the cells in the pharmaceutical composition are NKX6.1⁺/ISL1⁺ cells, as determined by flow cytometry. In some cases, no less than 30% of the cells in the composition are NKX6.1-positive, ISL1-positive cells, no less than 25% of the cells in the composition are NKX6.1-negative, ISL1-positive cells, less than 12% of the cells in the composition are NKX6.1-negative, ISL1-negative cells or between 9-25% of the cells in the composition are NKX6.1-positive, ISL1-negative cells as determined by flow cytometry. In some cases, no less than 40%, 35%, 30%, 26 %, 25%, or 20% of the cells in the pharmaceutical composition are NKX6.1⁻/ISL1⁺ cells, as determined by flow cytometry. In some cases, no less than 26 % of the cells in the pharmaceutical composition are NKX6.1⁻/ISL1⁺ cells, as determined by flow cytometry. In some cases, between 5-25%, 5-40%, 5-35%, or 8-20% of the cells in the pharmaceutical composition are NKX6.1⁻/ISL1⁺ cells, as determined by flow cytometry. In some cases, no more than 50%, 45%, 40%, 35%, 30%, or 25% of the cells in the pharmaceutical composition are NKX6.1⁺/ISL1⁻ cells, as determined by flow cytometry. In some cases, no more than 50% of the cells in the pharmaceutical composition are NKX6.1⁺/ISL1⁻ cells, as determined by flow cytometry.

In some cases, the population of cells are generated from stem cells in vitro. In some cases, the non-native cells expressing C-peptide and ISL1 but not VMAT1 exhibit glucose-stimulated insulin secretion response in vitro. In some cases, secretion of insulin by the non-native cells expressing C-peptide and ISL1 but not VMAT1 in response to a glucose challenge is proportional to glucose concentration of the glucose challenge. In some cases, secretion of insulin by the non-native cells expressing C-peptide and ISL1 but not VMAT1 secrete insulin in response to a first glucose challenge, a second glucose challenge, and a third glucose challenge, wherein the first glucose challenge, the second glucose challenge, and the third glucose challenge are applied sequentially.

In some cases, at least a portion of the cells in the population of cells are present in plurality of cell clusters. In some cases, the cell clusters are about 50 µm to about 500 µm, about 50 µm to about 400 µm, about 50 µm to about 300 µm, about 60 µm to about 400 µm, about 60 µm to about 300 µm, about 60 µm to about 250 µm, about 75 µm to about 400 µm, about 75 µm to about 300 µm, about 75 µm to about 250 µm, about 125 µm to about 225 µm, about 130 µm to about 160 µm, about 170 µm to about 225 µm, about 140 µm to about 200 µm, about 140 µm to about 170 µm, about 160 µm to about 220 µm, about 170 µm to about 215 µm, or about 170 µm to about 200 µm in diameter. In some cases, the population of cells are present as a single cell suspension in the pharmaceutical composition. In some cases, the population of cells is suspended in a serum-free solution.

Disclosed herein, in some aspects, is a kit, comprising: the pharmaceutical composition disclosed herein, and instruction for administering the pharmaceutical composition to a subject in need thereof.

Disclosed herein, in some aspects, is a kit, comprising: (a) a pharmaceutical composition formulated for infusion, wherein the pharmaceutical composition comprises a population of cells in suspension, wherein the population of cells comprises at least about 1 × 10⁸ cells, and wherein the population of cells comprises non-native cells expressing C-peptide and ISL1; and (b) instruction for administering the pharmaceutical composition to a subject in need thereof.

In some cases, the kit further comprises an immune response modulator. In some cases, the immune response modulator is not a steroid. In some cases, the immune response modulator comprises azathioprine, mycophenolic acid, leflunomide, teriflunomide, methotrexate, tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, anti-lymphocyte globulin antibodies, CTLA-4, abatacept, belatacept, etanercept, pegsunercept, aflibercept, alefacept, rilonacept, eculizumab, adalimumab, afelimomab, certolizumab pegol, golimumab, infliximab, nerelimomab, mepolizumab, omalizumab, faralimomab, elsilimomab, lebrikizumab, ustekinumab, secukinumab, muromonab-CD3, otelixizumab, teplizumab, visilizumab, clenoliximab, keliximab, zanolimumab, efalizumab, erlizumab, obinutuzumab, rituximab, ocrelizumab, pascolizumab, gomiliximab, lumiliximab, teneliximab, toralizumab, aselizumab, galiximab, gavilimomab, ruplizumab, belimumab, blisibimod, ipilimumab, tremelimumab, bertilimumab, lerdelimumab, metelimumab, natalizumab, tocilizumab, odulimomab, basiliximab, daclizumab, inolimomab, atorolimumab, cedelizumab, fontolizumab, maslimomab, morolimumab, pexelizumab, reslizumab, rovelizumab, siplizumab, talizumab, telimomab aritox, vapaliximab, vepalimomab, alemtuzumab, mycophenolate mofetil, FTY720, or any combination thereof. In some cases, the immune response modulator comprises tacrolimus, cyclosporine, mycophenolate mofetil, azathioprine, everolimus, sirolimus, abatacept, belatacept, antithymocyte globulin, alemtuzumab, rituximab, basiliximab, daclizumab, muromonab-CD3, efalizumab, FTY720, or any combination thereof. In some cases, the immune response modulator comprises a corticosteroid.

In some cases, the kit comprises instruction for administering the pharmaceutical composition to a subject who has a medical history of severe hypoglycemic events. In some cases, the kit comprises instruction for administering the pharmaceutical composition to a subject who has a medical history of impaired hypoglycemia awareness. In some cases, the kit comprises instruction for administering the pharmaceutical composition to a subject with no residual endogenous islet cell function. In some cases, the no residual endogenous islet cell function is indicated by undetectable fasting C-peptide and by undetectable stimulated C-peptide at the Mixed Meal Tolerance Test. In some cases, the no residual endogenous islet cell function is indicated by sustained stimulated glucose levels greater than 350, 400, 450, 475, or 500 mg/dL at the Mixed Meal Tolerance Test.

In some cases, the kit comprises instruction for administering the pharmaceutical composition to a subject who has an HbA1c level greater than 7.7%, 8%, 8.2%, or 8.5%. In some cases, the kit comprises instruction for administering the pharmaceutical composition to a subject who has been receiving greater than 10, 15, 20, 25, 30 or 35 units per day of insulin. In some cases, the kit comprises instruction for administering the pharmaceutical composition to a subject who has a disease characterized by high blood sugar levels over a prolonged period of time. In some cases, the disease is diabetes. In some cases, the disease is Type 1 diabetes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The file of this patent or application contains at least one drawing executed in color. Copies of this patent with color drawing(s) will be provided by the Patent and Trademark Office upon request and payment of the necessary fee.

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1A shows stimulated blood C-peptide level (top panel) and stimulated glucose level (bottom panel) of Subject A1 measured in an MMTT test during the screening period prior to infusion of the non-native pancreatic cells according to embodiments of the present disclosure. LLOQ: lower limit of quantification; MMTT: mixed-meal tolerance test. C-peptide values below the LLOQ (13 pmol/L [0.04 ng/mL]) were inputed as ½ LLOQ (7 pmol/L [0.02 ng/mL]). FIG. 1B shows stimulated blood C-peptide level (top panel) and stimulated glucose level (bottom panel) of Subject A2/B2 measured in an MMTT test during the screening period prior to infusion of the non-native pancreatic cells according to embodiments of the present disclosure. LLOQ: lower limit of quantification; MMTT: mixed-meal tolerance test. C-peptide values below the LLOQ (13 pmol/L [0.04 ng/mL]) were inputed as ½ LLOQ (7 pmol/L [0.02 ng/mL]). FIG. 1C shows stimulated blood C-peptide level (top panel) and stimulated glucose level (bottom panel) of Subject B1 measured in an MMTT test during the screening period prior to infusion of the non-native pancreatic cells according to embodiments of the present disclosure. LLOQ: lower limit quanitification; MMTT: mixed-meal tolerance test. C-peptide values below the LLOQ (13 pmol/L [0.04 ng/mL]) were inputed as ½ LLOQ (7 pmol/L [0.02 ng/mL]).

FIG. 2A is a plot summarizing the change of HbA1c of Subject A1 before and after receiving infusion of the non-native pancreatic cells according to embodiments of the present disclosure. B: Baseline; D: Day; HbA1c: hemoglobin A1c; M: Month; SCR: Screening; Unsch: unscheduled. FIG. 2B is a plot summarizing the change of HbA1c of Subject A2/B2 before and after receiving infusion of the non-native pancreatic cells according to embodiments of the present disclosure. B: Baseline; D: Day HbA1c: hemoglobin A1c; INF1: 1^{st} infusion of SC-islets; INF2: 2^{nd} infusion of SC-islets; SCR: Screening; Unsch: unscheduled. FIG. 2C is a plot summarizing the change of HbA1c of Subject B1 before and after receiving infusion of the non-native pancreatic cells according to embodiments of the present disclosure. B: Baseline; D: Day; HbA1c: hemoglobin A1c; SCR: Screening.

FIG. 3A is a bar graph summarizing time-in-range for Subject A1 measured by CGM according to embodiments of the present disclosure. Percentages of time for Days [301, 330] and Days [426, 455] are set as missing because <70% of data were available and there were no 14-consecutive-day periods where ≥70% of data were available. FIG. 3B is a bar graph summarizing time-in-range for Subject A2/B2 measured by CGM according to embodiments of the present disclosure. INF1: 1^{st} infusion of SC-islets; INF2: 2^{nd} infusion of SC-islets. On Day 249 after the first infusion, Subject A2/B2 stopped using CGM and an insulin pump for insulin delivery and began using a hybrid closed loop system (HCLS). FIG. 3C is a bar graph summarizing time-in-range for Subject B1 measured by CGM according to embodiments of the present disclosure.

FIG. 4A is a plot summarizing the change in the total daily exogenous insulin dose over time for Subject A1. FIG. 4B is a plot summarizing the change in the total daily exogenous insulin dose over time for Subject A2/B2. Total daily exogenous insulin dose data from baseline through Day 57 after the first infusion and from Day 150 through Day 210 after the first infusion are based on insulin pump data. Day 90 and Day 120 after the first infusion data are based on the eDiary. On Study Day 249, after the first infusion, Subject A2/B2 stopped using CGM and an insulin pump for insulin delivery and began using a hybrid closed loop system (HCLS). FIG. 4C is a plot summarizing the change in the total daily exogenous insulin dose over time for Subject B1. Total daily exogenous insulin dose data from baseline through Day 150 is based on insulin pump data. Day 180 data is based on eDiary.

### DETAILED DESCRIPTION

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic ε cells. Islet cells can also refer to a group of cells, cell clusters, or the like.

As used here, the term “pharmaceutically acceptable” can refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” can refer to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier” or the like are used interchangeably herein.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, when cultured under conditions suitable for differentiating the precursor cell into the insulin-positive endocrine cell.

The terms “stem cell-derived β cell,” “SC-β cell,” “functional β cell,” “functional pancreatic β cell,” “mature SC-β cell,” and their grammatical equivalents can refer to cells (e.g., non-native pancreatic β cells) that display at least one marker indicative of a pancreatic β cell (e.g., PDX-1 or NKX6.1) and expresses insulin. In some embodiments, the SC-β cells display a glucose stimulated insulin secretion (GSIS) response characteristic of an endogenous mature β cell. In some embodiments, the terms “SC-β cell” and “non-native β cell” as used herein are interchangeable. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the disclosure is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. The GSIS response of the SC-β cell can be observed within two weeks of transplantation of the SC-β cell into a host (e.g., a human or animal). In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose. In some embodiments, the SC-β cells express lower levels of MAFA than β cells from the pancreas of a healthy control adult subject. In some embodiments, the SC-β cells express higher levels of MAFB than β cells cells from the pancreas of a healthy control adult subject. In some embodiments, the SC-β cells express higher levels of SIX2, HOPX, IAPP and/or UCN3 than β cells cells from the pancreas of a healthy control adult subject. In some embodiments, the SC-β cells do not express MAFA. In some embodiments, the SC-β cells express MAFB. In some embodiments, any of the cell markers disclosed herein (e.g., MAFA, MAFB, SIX2, HOPX, IAPP and/or UCN3) are detected by flow cytometry. In some embodiments, the population comprises one or more NKX6.1-positive, ISL1-positive cells that express CHGA, MAFB, and/or ESRRG at a higher level (e.g., at least 10%, 30%, 50%, 70%, 100%, 125%, 150%, or 200% higher) than a NKX6.1-positive, ISL1-positive cell from the pancreas of a healthy control adult subject. In some embodiments, at least 30%, 40%, 50%, 60%, 70%, 80%, 90%, 95%, or 100% of the NKX6.1-positive, ISL1-positive cells in a population of cells express CHGA, MAFB, and/or ESRRG at a higher level than at least 30%, 40%, 50%, 60%, 70%, 80%, 90%, 95%, or 100% of the NKX6.1-positive, ISL1-positive cells from the pancreas of a healthy control adult subject. In some embodiments, the population comprises one or more NKX6.1-positive, ISL1-positive cells that express SIX3, MAFA, CHGB, RBP4 and/or FXYD2 at a lower level (e.g., at least 10%, 20%, 30%, 40%, 50%, 60%, 70%, 80%, 90% or 100% lower) than a NKX6.1-positive, ISL1-positive cell from the pancreas of a healthy control adult subject. In some embodiments, at least 30%, 40%, 50%, 60%, 70%, 80%, 90%, 95%, or 100% of the NKX6.1-positive, ISL1-positive cells in a population of cells express SIX3, MAFA, CHGB, RBP4 and/or FXYD2 at a lower level than at least 30%, 40%, 50%, 60%, 70%, 80%, 90%, 95%, or 100% of the NKX6.1-positive, ISL1-positive cells from the pancreas of a healthy control adult subject. In some embodiments, the population comprises NKX6.1-positive, ISL1-positive cells that express lower levels of SIX3 than NKX6.1-positive, ISL1-positive cells from the pancreas of a healthy control adult subject. In some embodiments, the population comprises NKX6.1-positive, ISL1-positive cells that express lower levels of CHGB than NKX6.1-positive, ISL1-positive cells from the pancreas of a healthy control adult subject. In some embodiments, the population comprises NKX6.1-positive, ISL1-positive cells that express lower levels of RBP4 than NKX6.1-positive, ISL1-positive cells from the pancreas of a healthy control adult subject. In some embodiments, the population comprises NKX6.1-positive, ISL1-positive cells that express lower levels of FXYD2 than NKX6.1-positive, ISL1-positive cells from the pancreas of a healthy control adult subject.

In some embodiments, any of the NKX6.1-positive, ISL1-positive cells disclosed herein also expresses any one or more of the following genes: PC2, MNX1, or ABCC8.

The terms “stem cell-derived α cell,” “SC-α cell,” “functional α cell,” “functional pancreatic α cell,” “mature SC-α cell,” and their grammatical equivalents can refer to cells (e.g., non-native pancreatic α cells) that display at least one marker indicative of a pancreatic α cell (e.g., glucagon, expressing ISL1 but not NKX6.1), expresses glucagon, and secretes functional glucagon. In some embodiments, the “SC-α cell” does not express somatostatin. In some embodiments, the “SC-α cell” does not express insulin. In some embodiments, the terms “SC-α cell” and “non-native α cell” as used herein are interchangeable. In some embodiments, the “SC-α cell” comprises a mature pancreatic cell.

The terms “stem cell-derived δ cell,” “SC-δ cell,” “functional δ cell,” “functional pancreatic δ cell,” “mature SC-δ cell,” and their grammatical equivalents can refer to cells (e.g., non-native pancreatic δ cells) that display at least one marker indicative of a pancreatic δ cell (e.g., somatostatin), expresses and secretes somatostatin. In some embodiments, “SC-δ cell” does not express glucagon. In some embodiments, “SC-δ cell” does not express insulin. In some embodiments, the terms “SC-δ cell” and “non-native δ cell” as used herein are interchangeable. In some embodiments, the “SC-δ cell” comprises a mature pancreatic cell.

The terms “stem cell-derived enterochromaffin (EC) cell,” “SC-EC cell,” and their grammatical equivalents can refer to cells (e.g., non-native pancreatic EC cells) that display at least one marker indicative of a pancreatic EC cell (e.g., VMAT1, expressing NKX6.1 but not ISL1). In some embodiments, the terms “SC-EC cell” and “non-native EC cell” as used herein are interchangeable.

Similar to SC-β cells, it is to be understood that the SC-α, SC-δ cells, and SC-EC cells need not be derived (e.g., directly) from stem cells, as the methods of the disclosure are capable of deriving SC-α cells from other precursor cells generated during in vitro differentiation of SC-β cells as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the disclosure is not intended to be limited in this manner).

The phrase “stem cell-derived islet cell” or “SC-islet cell” is an islet cell derived from a stem cell. Examples of SC-islet cells include SC-β cells, SC-α, SC-δ cells, and SC-EC cells. SC-islet cells are not mature islet cells obtained from a cadaver or from a human subject.

The phrase “therapeutically-effective amount” as used herein in respect to a population of cells means that amount of relevant cells in a population of cells, e.g., SC-β cells or mature pancreatic β cells, or composition comprising SC-β cells of the present disclosure which is effective for producing some desired therapeutic effect in at least a sub-population of cells in an animal at a reasonable benefit/risk ratio applicable to any medical treatment. For example, an amount of a population of SC-β cells administered to a subject that is sufficient to produce a statistically significant, measurable change in at least one symptom of Type 1, Type 1.5 or Type 2 diabetes, such as glycosylated hemoglobin level, fasting blood glucose level, hypoinsulinemia, etc. Determination of a therapeutically effective amount is well within the capability of those skilled in the art. Generally, a therapeutically effective amount can vary with the subject’s history, age, condition, sex, as well as the severity and type of the medical condition in the subject, and administration of other pharmaceutically active agents.

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic β-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive endocrine cells or a precursor thereof into SC-β cells according to the methods of the present disclosure can be pancreatic β cell or (β-like cells (e.g., cells that have at least one, or at least two least two) characteristic of an endogenous β cell and may exhibit a glucose stimulated insulin secretion (GSIS) response that resembles an endogenous adult β cell. The population of insulin-producing cells, e.g. produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive β-like cell,” “insulin-positive endocrine cell,” and their grammatical equivalents can refer to cells (e.g., pancreatic endocrine cells) that display at least one marker indicative of a pancreatic β cell and also expresses insulin but lack a glucose stimulated insulin secretion (GSIS) response characteristic of an endogenous β cell. Exemplary markers of “insulin-positive endocrine cell” include, but are not limited to, NKX6.1, ISL1, and insulin. In some cases, the terms “insulin-positive endocrine cell” and “NKX6.1-positive, ISL1-positive cell” are used interchangeably.

A “cell marker”, as used herein, refers without limitation to proteins, peptides, nucleic acids, polymorphism of proteins and nucleic acids, splice variants, fragments of proteins or nucleic acids, elements, and other analytes which are specifically expressed or present in one or more specific cell types and are not expressed or present in other specific cell types. For example, some of the cells in a population of cells may express PDX1, while other cells in the population do not. In particular embodiments, the presence or absence of one or more cell markers is determined using commercially available antibodies, e.g., in flow cytometry and/or in immunohistochemistry. In other embodiments, the presence or absence of one or more cell markers is detected using RT-PCR. Examples of cell markers include PDX1, NKX6.1, NGN-3, Neuro-D, ISL1, insulin, C-peptide, glucagon, somatostatin, VMAT1, MAFA, and MAFB. Other examples of specific cell markers are referenced throughout this disclosure.

The term “β cell marker” refers to, without limitation, proteins, peptides, nucleic acids, polymorphism of proteins and nucleic acids, splice variants, fragments of proteins or nucleic acids, elements, and other analytes which are specifically expressed or present in pancreatic β cells. Exemplary β cell markers may include, but are not limited to, pancreatic and duodenal homeobox 1 (PDX1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, and MafA, and those described in Zhang et al., Diabetes. 50(10):2231-6 (2001). In some embodiment, the β cell marker is a nuclear β-cell marker. In some embodiments, the β cell marker is PDX1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analyte which are specifically expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g. β cells that produce insulin; α cells that produce glucagon; δ cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “PDX1-positive pancreatic progenitor” or “PDX1-positive, NKX6.1-negative pancreatic progenitor” as used herein can refer to a cell which is a pancreatic endoderm (PE) cell which has the capacity to differentiate into SC-β cells, such as pancreatic β cells. A PDX1-positive pancreatic progenitor expresses the marker PDX1. Other markers include, but are not limited to Cdcp1, or Ptf1a, or HNF6 or NRx2.2. The expression of PDX1 may be assessed by any method known by the skilled person such as flow cytometry, immunochemistry using an anti-PDX1 antibody or quantitative RT-PCR. In some cases, a PDX1-positive pancreatic progenitor cell lacks expression of NKX6.1. In some cases, a PDX1-positive pancreatic progenitor cell can also be referred to as PDX1-positive, NKX6.1-negative pancreatic progenitor cell due to its lack of expression of NKX6.1. In some cases, the PDX1-positive pancreatic progenitor cells can also be termed as “pancreatic foregut endoderm cells.”

The terms “PDX1-positive, NKX6.1-positive pancreatic progenitor,” and “NKX6.1-positive pancreatic progenitor” are used interchangeably herein and can refer to a cell which is a pancreatic endoderm (PE) cell which has the capacity to differentiate into insulin-producing cells, such as pancreatic β cells. A PDX1-positive, NKX6.1-positive pancreatic progenitor expresses the markers PDX1 and NKX6-1. Other markers include, but are not limited to Cdcp1, or Ptf1a, or HNF6 or NRx2.2. The expression of NKX6-1 may be assessed by any method known by the skilled person such as flow cytometry, immunochemistry using an anti-NKX6-1 antibody or quantitative RT-PCR. As used herein, the terms “NKX6.1” and “NKX6-1” are equivalent and interchangeable. In some cases, the PDX1-positive, NKX6.1-positive pancreatic progenitor cells can also be termed as “pancreatic foregut precursor cells.”

The terms “NeuroD” and “NeuroD1” are used interchangeably and identify a protein expressed in pancreatic endocrine progenitor cells and the gene encoding it.

The term “epigenetics” refers to heritable changes in gene function that do not involve changes in the DNA sequence. Epigenetics most often denotes changes in a chromosome that affect gene activity and expression, but can also be used to describe any heritable phenotypic change that does not derive from a modification of the genome. Such effects on cellular and physiological phenotypic traits can result from external or environmental factors, or be part of normal developmental program. Epigenetics can also refer to functionally relevant changes to the genome that do not involve a change in the nucleotide sequence. Examples of mechanisms that produce such changes are DNA methylation and histone modification, each of which alters how genes are expressed without altering the underlying DNA sequence. Gene expression can be controlled through the action of repressor proteins that attach to silencer regions of the DNA. These epigenetic changes can last through cell divisions for the duration of the cell’s life, and can also last for multiple generations even though they do not involve changes in the underlying DNA sequence of the organism. One example of an epigenetic change in eukaryotic biology is the process of cellular differentiation. During morphogenesis, totipotent stem cells become the various pluripotent cells, which in turn can become fully differentiated cells.

The term “epigenetic modifying compound” refers to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic modification has a crucial role in the regulation of pluripotency genes, which become inactivated during differentiation. Non-limiting exemplary epigenetic modifying compounds include a DNA methylation inhibitor, a histone acetyltransferase inhibitor, a histone deacetylase inhibitor, a histone methyltransferase inhibitor, a bromodomain inhibitor, or any combination thereof.

The term “differentiated cell” or its grammatical equivalents is meant any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell may lead to the pancreatic pathway, where, in some embodiments, ~98% of the cells become exocrine, ductular, or matrix cells, and ~2% become endocrine cells. Early endocrine cells are islet progenitors, which can then differentiate further into insulin-producing cells (e.g. functional endocrine cells) which secrete insulin, glucagon, somatostatin, or pancreatic polypeptide. Endoderm cells can also be differentiated into other cells of endodermal origin, e.g. lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body - apart from the sperm and ova, the cells from which they are made (gametocytes) and undifferentiated stem cells - is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell is capable of differentiating to give rise first to the embryonic gut and then to the linings of the respiratory and digestive tracts (e.g. the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are capable of developing from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker PDX1 (e.g. they are PDX1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. In some cases, a primitive gut tube cell is FOXA2-positive and SOX2-positive, i.e., express both FOXA2 (also known as HNF3-β) and SOX2. In some cases, a primitive gut tube cell is FOXA2-positive and PDX1-negative, i.e., express FOXA2 but not PDX1. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “subject,” “patient,” or “individual” are used interchangeably herein, and can refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject can refer to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In some embodiments, administration is administration of one or more devices housing a cell population into a subject (e.g., subcutaneously and/or preperitoneally). In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

### Methods of Treatment

In some aspects, provided herein are methods for treating a disease characterized by high blood sugar levels over a prolonged period of time, e.g., diabetes, e.g., Type 1 diabetes in a subject. In some aspects, provided herein are methods for preventing a disease characterized by high blood sugar levels over a prolonged period of time, e.g., diabetes, e.g., Type 1 diabetes in a subject, e.g., a subject at risk of developing such a disease. In some aspects, provided herein are methods for reducing likelihood of developing a disease characterized by high blood sugar levels over a prolonged period of time, e.g., diabetes, e.g., Type 1 diabetes in a subject. A composition comprising a population of cells (e.g., cell clusters and/or cells) provided herein or generated according to the methods provided herein can be administered into a subject to restore a degree of pancreatic function in the subject. For example, the cell clusters resembling endogenous pancreatic islets, or the cells resembling endogenous pancreatic α, β and/or δ cells (e.g., non-native pancreatic α, β and/or δ cells) or the precursors thereof can be administered to a subject to treat, prevent, or reduce likelihood of developing a disease characterized by high blood sugar levels over a prolonged period of time, e.g., diabetes, e.g., Type 1 diabetes.

The methods can comprise administering the cell clusters or the cells disclosed in the application to a subject, e.g., a subject in need thereof, via transplantation of the cells in an encapsulation composition (e.g., a device). In some embodiments, the the subject is administered the cells/cell clusters in a device (e.g., subcutaneously or preperitoneally).

The methods can comprise administering the cell clusters or the cells disclosed in the application to a subject, e.g., a subject in need thereof, via infusion. The term “infusion” can refer to delivery of cells or cell clusters, any portion of the cells or cell clusters thereof, or any compositions comprising cells, cell clusters or any portion thereof, into the blood stream of a subject, for instance, e.g., into a vein of the subject, e.g., the hepatic portal vein, or an infusion port inserted into the blood stream.

In some embodiments, the method disclosed herein comprises administering to a subject via infusion a pharmaceutical composition disclosed herein. The pharmaceutical composition can comprise a population of cells (e.g., SC-islet cells) in a liquid suspension. The population of cells can comprise the cell clusters resembling endogenous pancreatic islets, or the cells resembling endogenous pancreatic α, β and/or δ cells (e.g., non-native pancreatic α, β and/or δ cells) or the precursors thereof. For instance, the population of cells can comprise non-native cells expressing C-peptide and ISL1. In some embodiments, the population of cells comprises non-native cells expressing C-peptide and ISL1 but not VMAT1. Additionally, the population of cells can comprise non-native cells expressing glucagon but not somatostatin (e.g., non-native α cell), and non-native cells expressing somatostatin but not glucagon (e.g., non-native δ cell).

As used herein, the term “treating” and “treatment” can refer to administering to a subject an effective amount of a composition (e.g., cell clusters or a portion thereof) so that the subject has a reduction in at least one symptom of the disease or an improvement in the disease, for example, beneficial or desired clinical results. In some embodiments, the subject become insulin-independent following treatment. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

By “treatment,” “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

A goal of diabetes treatment is to bring sugar levels down to as close to normal as is safely possible. Commonly set goals are 80-120 milligrams per deciliter (mg/dl) before meals and 100-140 mg/dl at bedtime. A particular physician may set different targets for the patient, depending on other factors, such as how often the patient has low or high blood sugar reactions. Useful medical tests include tests on the patient’s blood and urine to determine blood sugar level, tests for glycosylated hemoglobin level (HbA1c; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 1998). A successful treatment program can also be determined by having fewer patients in the program with complications relating to diabetes, such as severe hypoglycemic events, diseases of the eye, kidney disease, or nerve disease.

The methods of treatment disclosed herein can result in amelioration of one or more physiological parameters related to blood glucose regulation. In some cases, the methods disclosed herein can result in at least partial restoration of glucose-responsive insulin secretion in the subject. Without wishing to be bound by a certain theory, glucose-responsive insulin secretion in the subject restored by the methods and compositions disclosed herein can lead to amelioration of one or more symptoms or complications associated with diabetes in the subject. In some aspects, the disclosure relates to a method comprising administering to a subject a pharmaceutical composition comprising a population of cells provided herein (e.g., SC-islet cells), wherein the administered cells release insulin in an amount sufficient for a reduction of blood glucose levels in the subject.

After administration of the pharmaceutical composition according to embodiments of the present disclosure, blood C-peptide level in the subject can be increased to a level that at least partially ameliorates one or more symptoms of the disease, e.g., more stable, reduced blood glucose level, reduced hemoglobin A1c level, lowered reliance on intake of exteneral insulin (e.g., in order to stablize blood gloose level), or fewer severe hypoglycemic events. In some cases, the method elevates stimulated blood C-peptide level of the subject to at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, 550 pmol/L, 700 pmol/L, 800 pmol/L, 900 pmol/L, 1000 pmol/L, 1400 pmol/L, or between 150-350 pmol/L, 250-400 pmol/L, 400-1400 pmol/L, 400-1200 pmol/L, 400-1000 pmol/L, 400-800 pmol/L, 400-600 pmol/L, 600-800 pmol/L, 600-1400 pmol/L, or 900-1200 pmol/L, when measured at least about 1, 2, 3, 4, 5, or 6 months after the administration. The measurement of stimulated blood C-peptide level can be conducted using a standardized test, e.g., a mixed meal tolerance test, or an oral glucose tolerance test. In some cases, the method elevates stimulated blood C-peptide level of the subject to at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, or 550 pmol/L, 700 pmol/L, 800 pmol/L, 900 pmol/L, 1000 pmol/L, 1400 pmol/L or between 150-350 pmol/L, 250-400 pmol/L, 400-1400 pmol/L, 400-1200 pmol/L, 400-1000 pmol/L, 400-800 pmol/L, 400-600 pmol/L, 600-800 pmol/L, 600-1400 pmol/L, or 900-1200 pmol/L, when measured about 1, 2, 3, 4, 5, 6, 7, 8, 9, 10, 11, or 12 months after the administration. In some cases, the method elevates stimulated blood C-peptide level of the subject to at least at least 200 pmol/L, 300 pmol/L, 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, 550 pmol/L, 600 pmol/L, or 650 pmol/L, or between 150-350 pmol/L, 200-700 pmol/L, 150-700 pmol/L, 300-650 pmol/L, or 500-700 pmol/L, when measured about 3 months after the administration. In some cases, the method elevates stimulated blood C-peptide level of the subject to at least 450 pmol/L, 460 pmol/L, 470 pmol/L, 480 pmol/L, 490 pmol/L, 500 pmol/L, 510 pmol/L, 520 pmol/L, 530 pmol/L, 540 pmol/L, or 550 pmol/L, when measured about 5 months after the administration. In some embodiments, the method elevates stimulated blood C-peptide level of the subject to about 480 pmol/L, 500 pmol/L, 520 pmol/L, 540 pmol/L, or 560 pmol/L, when measured about 3 months after the administration. In some embodiments, the method elevates stimulated blood C-peptide level of the subject to about 480 pmol/L, 500 pmol/L, 520 pmol/L, 540 pmol/L, or 560 pmol/L, when measured about 5 months after the administration. In some embodiments, the stimulated blood C-peptide level of the treated subject is between 480 pmol/L and 560 pmol/L, when measured about 1, 2, 3, 4, 5, 6, 7, 8, 9, 10, 11, or 12 months after the administration (e.g., 3 or 5 months after the administration).

In some cases, oral glucose tolerance test or mixed meal tolerance test is performed in the morning following an overnight fast of >8 hours. The subject may be limited on strenuous exercise, alcohol, caffeine and tobacco use, all of which may influence insulin sensitivity. Mixed meal tolerance test can be conducted by following a protocol provided with a standardized meal of specified macronutrient content or a proprietary meal substitute, e.g., Ensure® (Abbott) or Boost ® Complete Nutritional Drink High Protein (Nestlé Health Science). For oral glucose tolerance test, a 75 g anhydrous glucose dissolved in 250 ml water may be used as a reference method.

In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured at least about 1, 2, 3, 4, 5, or 6 months after the administration. In some embodiments, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured about 1, 2, 3, 4, 5, 6, 7, 8, 9, 10, 11, or 12 months after the administration. In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured about 3 months after the administration. In some cases, the method elevates blood C-peptide level of the subject under fasting condition to at least 100 pmol/L, 120 pmol/L, 130 pmol/L, 150 pmol/L, 160 pmol/L, 180 pmol/L, 200 pmol/L, 210 pmol/L, 220 pmol/L, 230 pmol/L, 240 pmol/L, 250 pmol/L, 260 pmol/L, 270 pmol/L, or 280 pmol/L when measured about 5 months after the administration. In some cases, the method elevates blood C-peptide level of the subject under fasting condition to about 200 pmol/L, 220 pmol/L, 240 pmol/L, 260 pmol/L, or 280 pmol/L, when measured about 3 months after the administration. In some cases, the method elevates blood C-peptide level of the subject under fasting condition to about 200 pmol/L, 220 pmol/L, 240 pmol/L, 260 pmol/L, or 280 pmol/L, when measured about 5 months after the administration. In some cases, administration of a pharmaceutical composition according to the present disclosure reduces hemoglobin A1c level of the subject. For instance, the subject’s Hb1Ac level can be reduced to less than 8%, 7.5%, 7.0%, 6.5%, 6.0%, 5.5% or between 5-8%, 5-7.5%, 5-6.5%, 5-5.5%, 5.5-8%, 5.5-7%, 5.5-6.5%, 6-8%, 6-7%, or 6-6.5%, when measured at least about 1, 2, 3, 4, 5, or 6 months after the administration. In some cases, the subject’s Hb1Ac level is reduced to less than 8%, 7.5%, 7.0%, 6.5%, 6.0%, 5.5% or 5% or between 5-8%, 5-7.5%, 5-6.5%, 5-5.5%, 5.5-8%, 5.5-7%, 5.5-6.5%, 6-8%, 6-7%, or 6-6.5%,, when measured about 1, 2, 3, 4, 5, 6, 7, 8, 9, 10, 11, or 12 months after the administration. In some cases, the treated subject’s Hb1Ac level is 7-7.9% when measured about 1, 2, 3, 4, 5, 6, 7, 8, 9, 10, 11, or 12 months after the administration. In some cases, it is reduced to less than 8%, 7.5%, 7.0%, or 6.75% or between 5-8.0%, 6.5-8.0%, 6.5-7.5%, 6.5-7.0%, or 7-8%, when measured about 3 months after the administration. In some cases, it is reduced to less than 8%, 7.9%, 7.8%, 7.7%, 7.6%, 7.5%, 7.3%, 7.2%, 7.1%, or 7.0%, when measured about 5 months after the administration. In some cases, it is reduced to less than 8%, 7.5%, 7.0%, 6.5%, 6.0%, 5.5%, or 5%, or between 4.5-8%, 5-8%, 5-7.5%, 5-7%, 5-6.5%, 5-5.5%, 5.5-8%, 5.5-7%, 5.5-6.5%, 6-8%, 6-7%, or 6-6.5%, when measured about 9 months after the administration. In some cases, it is reduced to about 7.6%, 7.5%, 7.4%, 7.3%, 7.2%, 7.1%, or 7.0%, when measured about 5 months after the administration.

Patients with a disease characterized by high blood glucose levels, e.g., diabetes, may have a medical need to receive infusion of external insulin to manage blood glucose level (“glycemic control”). Typically, external insulin is administered to such patients with a predeteremined goal for glycemic control, e.g., to maintain blood glucose level within a certain range, such as those discussed in Louis Monnier and Claude Colette, Target for Glycemic Control, Diabetes Care November 2009, 32 (suppl 2) S199-S204; DOI: 10.2337/dc09-S310, which is incorporated herein by its entirety. In some cases, the subject may be prescribed by a medical doctor with a personalized glycemic control goal to guide his/her insulin intake. The method disclosed herein can reduce the subject’s reliance on intake of external insulin while realizing his/her goal for glycemic control. In some cases, at least about 1, 2, 3, 4, 5, or 6 months after the administration, the subject’s daily insulin is reduced to at most 10 units (U), 9 U, 8 U, 7 U, 6 U, 5 U, 4 U, 3 U, or 2 U in average over a 2-day, 3-day, 4-day, 5-day, 6-day, 7-day, 8-day, 9-day, or 10-day period, or over a period of at least 10 days, 15 days, 20 days, or a month. In some cases, at least about 1, 2, 3, 4, 5, or 6 months after the administration, the subject’s daily insulin is reduced to about 10 units (U), 9 U, 8 U, 7 U, 6 U, 5 U, 4 U, 3 U, 2 U, or 1 U in average over a 2-day, 3-day, 4-day, 5-day, 6-day, 7-day, 8-day, 9-day, or 10-day period, or over a period of at least 10 days, 15 days, 20 days, or a month. In some cases, about 1, 2, 3, 4, 5, 6, 7, 8, 9, 10, 11, or 12 months after the administration, the subject does not need to take insulin infusion over a 2-day, 3-day, 4-day, 5-day, 6-day, 7-day, 8-day, 9-day, or 10-day period, or over a period of at least 10 days, 15 days, 20 days, or a month.

In some embodiments, the reduction of blood glucose levels in the subject, as induced by administration of the pharmaceutical composition provided herein, results in an amount of glucose which is lower than the diabetes threshold. In some embodiments, the subject is a mammalian subject. In some embodiments, the mammalian subject is human. In some embodiments, the amount of glucose is reduced to lower than the diabetes threshold in 1, 2, 3, 4, 5, 6, 7, 8, 9, or 10 days after the administration.

A subject that can be treated by the methods herein can be a human or a non-human animal. In some cases, a subject can be a mammal. Examples of a subject include but are not limited to primates, e.g., a monkey, a chimpanzee, a baboon, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow, but are not limited to these examples. Mammals other than humans can be advantageously used as subjects that represent animal models of Type 1 diabetes, Type 2 Diabetes Mellitus, or pre-diabetic conditions. In addition, the methods described herein can be used to treat domesticated animals and/or pets. A subject can be male or female. A subject can be one who has been previously diagnosed with or identified as suffering from or having diabetes (e.g., Type 1 or Type 2), one or more complications related to diabetes, or a pre-diabetic condition, and optionally, but need not have already undergone treatment for the Diabetes, the one or more complications related to diabetes, or the pre-diabetic condition. A subject can also be one who is not suffering from diabetes or a pre-diabetic condition. A subject can also be one who has been diagnosed with or identified as suffering from diabetes, one or more complications related to diabetes, or a pre-diabetic condition, but who show improvements in known diabetes risk factors as a result of receiving one or more treatments for diabetes, one or more complications related to diabetes, or the pre-diabetic condition. Alternatively, a subject can also be one who has not been previously diagnosed as having diabetes, one or more complications related to diabetes, or a pre-diabetic condition. For example, a subject can be one who exhibits one or more risk factors for diabetes, complications related to diabetes, or a pre-diabetic condition, or a subject who does not exhibit Diabetes risk factors, or a subject who is asymptomatic for diabetes, one or more diabetes-related complications, or a pre-diabetic condition. A subject can also be one who is suffering from or at risk of developing diabetes or a pre-diabetic condition. A subject can also be one who has been diagnosed with or identified as having one or more complications related to diabetes or a pre-diabetic condition as defined herein, or alternatively, a subject can be one who has not been previously diagnosed with or identified as having one or more complications related to diabetes or a pre-diabetic condition.

In some embodiments, prior to administration of any of the pharmaucetical compositions disclosed herein, the subject has stimulated blood C-peptide level of less than 100 pmol/L, when measured using a mixed meal tolerance test, such as less than 80 pmol/L, 60 pmol/L, 40 pmol/L, 30 pmol/L, 20 pmol/L, or 10 pmol/L. In some embodiments, prior to administration of the pharmaucetical composition disclosed herein, the subject has stimulated blood C-peptide level of less than 100 pmol/L, when measured using an oral glucose tolerance test, such as less than 80 pmol/L, 60 pmol/L, 40 pmol/L, 30 pmol/L, 20 pmol/L, or 10 pmol/L. In some cases, prior to the administration, the subject has undectable stimulated blood C-peptide level, e.g., when measured using a mixed meal tolerance test or using oral glucose tolerance test. In some cases, prior to the administration, the subject has undectable stimulated blood C-peptide level when measured under fasting condition. In some embodiments, prior to the administration, the subject presented with no residual endogenous islet cell function. In some embodiments, the no residual endogenous islet cell function is indicated by undetectable fasting C-peptide and undetectable stimulated C-peptide at the Mixed Meal Tolerance Test. In some embodiments, the no residual endogenous islet cell function is indicated by sustained stimulated glucose levels greater than 350, 400, 450, 475, or 500 mg/dL. In some embodiments, prior to the administration, the subject was receiving greater than 10, 15, 20, 25, 30 or 35 units per day of insulin. In some cases, prior to the administration, the subject has a baseline plama level of hemoglobin A1c of more than 8.0%, such as more than 8.1%, 8.2%, 8.3%, 8.4%, 8.5%, 8.6%, 8.8%, or 9.0%. In some embodiments, prior to the administration, the subject has an HbA1c level greater than 7.7%, 8%, 8.2%, or 8.5%. In some cases, prior to the administration, the subject receives infusion of insulin at a level of at least about 20 U/day, such as at least about 22 U/day, 24 U/day, 26 U/day, 28 U/day, 30 U/day, 32 U/day, or 34 U/day. In some cases, prior to the administration, the subject has a medical history of severe hypoglycemic events, for instance, the subject may have at least 1 severe hypoglycemic event every month, such as at least 2, 3, 4, 5, 6, 7, 8, 9, 10, 12, 15, or 20 severe hypoglycemic events. “Severe hypoglycemic event” can refer to an episode in which the person with diabetes requires the assistance of another to increase blood glucose, usually by administration of glucagon or contacting a medical professional. By depriving the brain of glucose, severe hypoglycemia can acutely alter brain function, resulting in neuroglycopenic symptoms, seizures, or even death. In some cases, prior to the administration, the subject has a medical history of impared hypoglycemia awareness.

In some cases, the methods of treatment provided herein can comprise administering one or more immune response modulators for modulating or reducing transplant rejection response or other immune response against the administered cells. In some embodiments, the immune response modulator is not or does not comprise a steroid. In some cases, the immune response modulator comprises a steroid such as corticosteroid. Examples of immune response modulators that can be used in the methods can include purine synthesis inhibitors like azathioprine and mycophenolic acid, pyrimidine synthesis inhibitors like leflunomide and teriflunomide, antifolate like methotrexate, tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, antilymphocyte globulin antibodies, CTLA-4, fragment thereof, and fusion proteins thereof like abatacept and belatacept, TNF inhibitor like etanercept and pegsunercept, aflibercept, alefacept, rilonacept, antibodies against complement component 5 like eculizumab, anti-TNF antibodies like adalimumab, afelimomab, certolizumab pegol, golimumab, infliximab, and nerelimomab, antibodies against Interleukin 5 like mepolizumab, anti-Ig E antibodies like omalizumab, anti-Interferon antibodies like faralimomab, anti-IL-6 antibodies like elsilimomab, antibodies against IL-12 and IL-23 like lebrikizumab and ustekinumab, anti-IL-17A antibodies like secukinumab, anti-CD3 antibodies like muromonab-CD3, otelixizumab, teplizumab, and visilizumab, anti-CD4 antibodies like clenoliximab, keliximab, and zanolimumab, anti-CD11a antibodies like efalizumab, anti-CD18 antibodies like erlizumab, anti-CD20 antibodies like obinutuzumab, rituximab, ocrelizumab and pascolizumab, anti-CD23 antibodies like gomiliximab and lumiliximab, anti-CD40 antibodies like teneliximab and toralizumab, antibodies against CD62L/L-selectin like aselizumab, anti-CD80 antibodies like galiximab, anti-CD147/Basigin antibodies like gavilimomab, anti-CD154 antibodies like ruplizumab, anti-BLyS antibodies like belimumab and blisibimod, anti-CTLA-4 antibodies like ipilimumab and tremelimumab, anti-CAT antibodies like bertilimumab, lerdelimumab, and metelimumab, anti-Integrin antibodies like natalizumab, antibodies against Interleukin-6 receptor like tocilizumab, anti-LFA-1 antibodies like odulimomab, antibodies against IL-2 receptor/CD25 like basiliximab, daclizumab, and inolimomab, antibodies against T-lymphocyte (Zolimomab aritox) like atorolimumab, cedelizumab, fontolizumab, maslimomab, morolimumab, pexelizumab, reslizumab, rovelizumab, siplizumab, talizumab, telimomab aritox, vapaliximab, and vepalimomab, antibodies against CD52 like alemtuzumab, blockers of inosine monophosphate dehydrogenase (IMPDH) like mycophenolate mofetil, inhibitors of cell emigration like FTY720.

In some embodiments, the subject administered any of the pharmaceutical compositions disclosed herein in accordance with any of the methods disclosed herein is also administered an immunosuppressant. In some embodiments, the immunosuppressant is selected from the group consisting of Thymoglobulin, Etanercept, Basiliximab, Tacrolimus, Sirolimus, and Mycophenolate mofetil. In some embodiments, the subject administered the SC-islet cells is also administered Thymoglobulin and Etanercept. In some embodiments, the subject administered the SC-islet cells is also administered Thymoglobulin and Basiliximab. In some embodiments, the subject administered the SC-islet cells is also administered Tacrolimus and Sirolimus. In some embodiments, the subject administered the SC-islet cells is also administered Tacrolimus and Basiliximab. In some embodiments, the subject administered the SC-islet cells is also administered Sirolimus and Mycophenolate. In some embodiments, the subject administered the SC-islet cells is also administered mycophenolate mofetil or mycophenolate sodium.

In some embodiments, the disclosure provides for administering to a subject a first pharmaceutical composition comprising between 1 × 10⁸ cells and 12 × 10⁸ (e.g., 8 × 10⁸ to 12 × 10⁸) SC-islet cells. In some embodiments, the disclosure provides for administering to a subject a first pharmaceutical composition comprising between 1 × 10⁸ cells and 12 × 10⁸ (e.g., 8 × 10⁸ to 12 × 10⁸) SC-islet cells, in a single administration (i.e,. in a single infusion, or in a single surgical procedure implanting one or more devices into one or more incision sites in the subject). In some embodiments, the disclosure provides for administering to a subject a first pharmaceutical composition comprising between 1 × 10⁸ cells and 12 × 10⁸ (e.g., 8 × 10⁸ to 12 × 10⁸) SC-islet cells, in multiple administrations (i.e,.in two or more infusions, or in multiple surgical procedures implanting one or more devices into one or more incision sites in the subject). In some embodiments, the disclosure provides for administering to a subject a first pharmaceutical composition comprising between 1 × 10⁸ cells and 10 × 10⁸ (e.g., 3 × 10⁸ to 8.5 × 10⁸) SC-islet cells. In some embodiments, the first pharmaceutical composition comprises at least about 1 × 10⁸ SC-islet cells, such as at least about 1.5 × 10⁸, at least about 2 × 10⁸, at least about 2.5 × 10⁸, at least about 3 × 10⁸, at least about 3.5 × 10⁸, at least about 4 × 10⁸, at least about 4.5 × 10⁸, at least about 5 × 10⁸, at least about 5.5 × 10⁸, at least about, at least about 6 × 10⁸, at least about 6.5 × 10⁸, or at least about 7 × 10⁸ SC-islet cells. In some cases, the first pharmaceutical composition disclosed herein comprises at most about 8 × 10⁸, at most about 7 × 10⁸, at most about 6.5 × 10⁸, at most about 6 × 10⁸, at most about 5.5 × 10⁸, at most about 5 × 10⁸, at most about 4.5 × 10⁸, at most about 4 × 10⁸, at most about 3.5 × 10⁸, at most about 3 × 10⁸, at most about 2.5 × 10⁸, or at most about 2 × 10⁸ SC-islet cells. In some embodiments, the first pharmaceutical composition comprises about 1 × 10⁸ to about 8 × 10⁸, about 1 × 10⁸ to about 7 × 10⁸, about 1 × 10⁸ to about 6 × 10⁸, about 1 × 10⁸ to about 5 × 10⁸, about 1 × 10⁸ to about 4 × 10⁸, about 1 × 10⁸ to about 3 × 10⁸, about 1 × 10⁸ to about 2 × 10⁸, about 1.5 × 10⁸ to about 6.5 × 10⁸, about 2 × 10⁸ to about 7 × 10⁸, about 2 × 10⁸ to about 6 × 10⁸, about 2 × 10⁸ to about 5 × 10⁸, about 2 × 10⁸ to about 4 × 10⁸, about 2 × 10⁸ to about 3 × 10⁸, about 2.5 × 10⁸ to about 5.5 × 10⁸, about 3 × 10⁸ to about 7 × 10⁸, about 3 × 10⁸ to about 6 × 10⁸, about 3 × 10⁸ to about 5 × 10⁸, about 3 × 10⁸ to about 4 × 10⁸, about 3.5 × 10⁸ to about 4.5 × 10⁸, or about 3.8 × 10⁸ to about 4.2 × 10⁸ SC-islet cells. In some cases, the first pharmaceutical composition comprises about 3.5 × 10⁸ to about 4.5 × 10⁸ SC-islet cells. In some embodiments, the first pharmaceutical composition comprises about 3.5 × 10⁸ to about 8.5 × 10⁸ SC-islet cells. In some embodiments, the first pharmaceutical composition comprises about 3.9 × 10⁸ to about 4.1 × 10⁸ SC-islet cells. In some embodiments, the first pharmaceutical composition comprises about 7.9 × 10⁸ to about 8.1 × 10⁸ SC-islet cells. In some embodiments, the first pharmaceutical composition comprises about 7.5 × 10⁸ to 12 × 10⁸ SC-islet cells (e.g., 7.5 × 10⁸ to 10 × 10⁸ SC-islet cells). In some embodiments, the subject receives only a single administration (e.g., via infusion or via one or more of any of the devices disclosed herein) of the pharmaceutical composition, wherein the composition comprises between 7 × 10⁸ and 14 × 10⁸, between 8 × 10⁸ and 13 × 10⁸, between 9 × 10⁸ and 12 × 10⁸, between 9 × 10⁸ and 11 × 10⁸, or between 9 × 10⁸ and 10 × 10⁸ SC-islet cells. In some embodiments, the subject is administered the cells in a single administration (i.e,. in a single infusion, or in a single surgical procedure implanting one or more devices into one or more incision sites in the subject).

In some embodiments, the subject is administered a first pharmaceutical composition comprising SC-islet cells, and the subject is not administered a further pharmaceutical composition comprising SC-islet cells (i.e., the first pharmaceutical composition comprising SC-islet cells administered to the subject is the only pharmaceutical composition comprising SC-islet cells administered to the subject). In some embodiments, the subject is administered a first pharmaceutical composition comprising about 3.5 × 10⁸ to about 4.5 × 10⁸ SC-islet cells, about 3.9 × 10⁸ to about 4.1 × 10⁸ SC-islet cells, 7.5 × 10⁸ to about 8.5 × 10⁸ SC-islet cells, about 7.5 × 10⁸ to about 9 × 10⁸ SC-islet cells, about 7.5 × 10⁸ to about 10 × 10⁸ SC-islet cells, about 10 × 10⁸ to about 12 × 10⁸ SC-islet cells, about 7.9 × 10⁸ to about 8.1 × 10⁸ SC-islet cells, about 4.0 × 10⁸ SC-islet cells, or about 8.0 × 10⁸ SC-islet cells, and the subject is not administered a further pharmaceutical composition comprising SC-islet cells. In some embodiments, the subject is administered a first pharmaceutical composition comprising SC-islet cells, and the subject is not administered a further pharmaceutical composition comprising SC-islet cells within 3, 4, 5, 6, 7, 8, 9 or 10 years of having received the first pharmaceutical composition comprising SC-ilset cells. In some embodiments, the subject is administered a first pharmaceutical composition comprising SC-islet cells, and the subject is not administered a further pharmaceutical composition comprising SC-islet cells within 5 years of having received the first pharmaceutical composition comprising SC-ilset cells. In some embodiments, the subject is administered a first pharmaceutical composition comprising SC-islet cells, and the subject is not administered a further pharmaceutical composition comprising SC-islet cells within 10 years of having received the first pharmaceutical composition comprising SC-ilset cells.

In some embodiments, the subject is administered a first pharmaceutical composition comprising SC-islet cells, and the subject is not administered a further pharmaceutical composition comprising SC-islet cells. In some embodiments, the subject is administered one or more pharmaceutical compositions comprising SC-islet cells in a single surgical procedure (e.g., to implant one or more devices housing SC-islet cells), and the subject is not administered any further pharmaceutical composition comprising SC-islet cells. In some embodiments, the subject is administered a first pharmaceutical composition comprising SC-islet cells, and the subject is administered at least a second pharmaceutical composition comprising SC-islet cells. In some embodiments, the subject is administered one or more pharmaceutical compositions comprising SC-islet cells in a first surgical procedure (e.g., to implant one or more devices housing SC-islet cells), and the subject is further administered one or more pharmaceutical compositions comprising SC-islet cells in a second surgical procedure (e.g., to implant one or more devices housing SC-islet cells). In some embodiments, the subject is administered a second pharmaceutical composition comprising SC-islet cells, but is not administered a third pharmaceutical composition comprising SC-islet cells in a one year period. In some embodiments, the subject is administered one or more pharmaceutical compositions comprising SC-islet cells in a first surgical procedure (e.g., to implant one or more devices housing SC-islet cells), and the subject is further administered one or more pharmaceutical compositions comprising SC-islet cells in a second surgical procedure (e.g., to implant one or more devices housing SC-islet cells), but the subject is not further administered any pharmaceutical compositions comprising SC-islet cells in a one year period. In some embodiments, the subject is administered a first pharmaceutical composition comprising about 3.5 × 10⁸ to about 4.5 × 10⁸ SC-islet cells, about 3.9 × 10⁸ to about 4.1 × 10⁸ SC-islet cells, or about 4.0 × 10⁸ SC-islet cells and the subject is administered at a later point in time a second pharmaceutical composition comprising about 3.5 × 10⁸ to about 4.5 × 10⁸ SC-islet cells, about 3.9 × 10⁸ to about 4.1 × 10⁸ SC-islet cells, or about 4.0 × 10⁸ SC-islet cells. In some embodiments, the second pharmaceutical composition is administered to the subject at least 3 months, 4 months, 5 months, 6 months, 7 months, 8 months, 9 months, 10 months, 11 months, 12 months, or 15 months or between 3-12 months, 3-10 months, 3-9 months, 3-7 months, 3-5 months, 5-12 months, 8-10 months, 7-12 months, 9-15 months, or 9-12 months after the subject is administered the first pharmaceutical composition.

In some embodiments, the subject is administered a first pharmaceutical composition comprising about 3.5 × 10⁸ to about 8.5 × 10⁸ SC-islet cells, about 3.5 × 10⁸ to about 4.5 × 10⁸ SC-islet cells, about 3.9 × 10⁸ to about 4.1 × 10⁸ SC-islet cells, 7.5 × 10⁸ to about 8.5 × 10⁸ SC-islet cells, about 7.9 × 10⁸ to about 8.1 × 10⁸ SC-islet cells, about 4.0 × 10⁸ SC-islet cells, or about 8.0 × 10⁸ SC-islet cells, and the subject is administered a second pharmaceutical composition comprising about 3.5 × 10⁸ to about 8.5 × 10⁸ SC-islet cells, about 3.5 × 10⁸ to about 4.5 × 10⁸ SC-islet cells, about 3.9 × 10⁸ to about 4.1 × 10⁸ SC-islet cells, 7.5 × 10⁸ to about 8.5 × 10⁸ SC-islet cells, about 7.9 × 10⁸ to about 8.1 × 10⁸ SC-islet cells, about 4.0 × 10⁸ SC-islet cells, or about 8.0 × 10⁸ SC-islet cells. In some embodiments, the second pharmaceutical composition is administered at at least 3, 4, 5, 6, 7, 8, 9 or 10 years or between 3-20, 10-20, 15-20, 5-15, 3-5, 5-8, 8-10, 10-12, 12-15, or 20-25 years after the first pharmaceutical composition is administered to the subject.

### Pharmaceutical Compositions

In some aspects, provided herein are pharmaceutical compositions that comprise a population of cells in a liquid suspension. In some cases, the pharmaceutical compositions disclosed herein are formulated for infusion. In some cases, the population of cells includes non-native cells that express C-peptide and ISL, e.g., SC-β cells. In some cases, the population of cells also includes non-native cells that express glucagon but not somatostatin, e.g., SC-α cells. In some cases, the population of cells also includes non-native cells expressing somatostatin but not glucagon, e.g., SC-δ cells. In some cases, the population of cells also includes non-native cells expressing VMAT1, e.g., SC-EC cells. In some cases, the cell constituent of the pharmaceutical compositions disclosed herein resembles a native pancreatic islet. The non-native pancreatic cells in the pharmaceutical compositions disclosed herein can be obtained by an in vitro differentiation process following the method of differentiation according to some embodiments of the present disclosure.

The pharmaceutical composition can be used to treat, prevent, or stabilize diabetes. For example, somatic cells or stem cells can be obtained from an individual in need of treatment or from a healthy individual and reprogrammed to stem cell derived beta cells by the method of the present disclosure. In one embodiment of the present disclosure the stem cell derived beta cells, and in some cases, other stem cell derived pancreactic cells (e.g., SC-α cells, SC-δ cells, SC-EC cells) are formulated according to the present disclosure and administered into the individual to treat the condition. In another embodiment the stem cells are cultured under conditions suitable for differentiation into beta cells prior to introduction into the individual, and can be used to replace or assist the normal function of diseased or damaged tissue. The great advantage of the present disclosure is that it provides an essentially limitless supply of patient specific human beta cells or compatible stem cell derived beta cells from healthy individuals suitable for transplantation. The use of autologous and/or compatible cells in cell therapy offers a major advantage over the use of non-autologous cells, which are likely to be subject to immunological rejection. In contrast, autologous cells are unlikely to elicit significant immunological responses.

In some embodiments, the pharmaceutical composition disclosed herein comprises from about 1 × 10⁸ cells to about 12 × 10⁸ (e.g., 3 × 10⁸ to 8.5 × 10⁸) cells. In some embodiments, the pharmaceutical composition comprises at least about 1 × 10⁸ cells, such as at least about 1.5 × 10⁸, at least about 2 × 10⁸, at least about 2.5 × 10⁸, at least about 3 × 10⁸, at least about 3.5 × 10⁸, at least about 4 × 10⁸, at least about 4.5 × 10⁸, at least about 5 × 10⁸, at least about 5.5 × 10⁸, at least about, at least about 6 × 10⁸, at least about 6.5 × 10⁸, or at least about 7 × 10⁸ cells the population of cells. In some cases, the pharmaceutical composition disclosed herein comprises at most about 8 × 10⁸, at most about 7 × 10⁸, at most about 6.5 × 10⁸, at most about 6 × 10⁸, at most about 5.5 × 10⁸, at most about 5 × 10⁸, at most about 4.5 × 10⁸, at most about 4 × 10⁸, at most about 3.5 × 10⁸, at most about 3 × 10⁸, at most about 2.5 × 10⁸, or at most about 2 × 10⁸ cells in the population of cells. In some embodiments, the population of cells in the pharmaceutical composition comprises about 1 × 10⁸ to about 8 × 10⁸, about 1 × 10⁸ to about 7 × 10⁸, about 1 × 10⁸ to about 6 × 10⁸, about 1 × 10⁸ to about 5 × 10⁸, about 1 × 10⁸ to about 4 × 10⁸, about 1 × 10⁸ to about 3 × 10⁸, about 1 × 10⁸ to about 2 × 10⁸, about 1.5 × 10⁸ to about 6.5 × 10⁸, about 2 × 10⁸ to about 7 × 10⁸, about 2 × 10⁸ to about 6 × 10⁸, about 2 × 10⁸ to about 5 × 10⁸, about 2 × 10⁸ to about 4 × 10⁸, about 2 × 10⁸ to about 3 × 10⁸, about 2.5 × 10⁸ to about 5.5 × 10⁸, about 3 × 10⁸ to about 7 × 10⁸, about 3 × 10⁸ to about 6 × 10⁸, about 3 × 10⁸ to about 5 × 10⁸, about 3 × 10⁸ to about 4 × 10⁸, about 3.5 × 10⁸ to about 4.5 × 10⁸, about 3.8 × 10⁸ to about 4.2 × 10⁸ cells, about 7.5 × 10⁸ to about 10 × 10⁸ cells, or about 8 × 10⁸ to about 12 × 10⁸ cells. In some cases, the population of cells in the pharmaceutical composition comprises about 3.5 × 10⁸ to about 4.5 × 10⁸ cells. In some embodiments, the population of cells in the pharmaceutical composition comprises about 3.5 × 10⁸ to about 8.5 × 10⁸ cells. In some embodiments, the population of cells in the pharmaceutical composition comprises about 3.9 × 10⁸ to about 4.1 × 10⁸ cells. In some embodiments, the population of cells in the pharmaceutical composition comprises about 7.9 × 10⁸ to about 8.1 × 10⁸ cells.

In some embodiments, to formulate the dose, a cell cluster suspension is first counted as dissociated single cells using an optical method that detects individual cells by phase contrast. After cells are counted in the suspension, the pelleted stock of cells may be adjusted to the appropriate volume and formulated in a solution (e.g., hyopthermasol) to a desired concentration (e.g., 2 million cells per milliliter for a total volume). For example, if the target dose is 430e6 cells, 215 mL of hypothermasol may be added.

In one embodiment, to achieve a targeted dose, two 0.5 mL samples are taken from the pre-formulated harvested drug substance for cell count and viability. Gross weight of the pooled cell suspension may be taken of pre-formulated drug substance once in the infusion medium and Total volume of Cell suspension is calculated with the Tared Tube starting mass. In order for material release, cells may be subject to cell count and viability via hemocytomter. Trypan exclusion method, in tandem with a disposable Neubauer hemocytometer, may be utilized to assess cell density of the pooled drug substance suspension. This method may be qualified to accurately count cells in a single cell suspension within concentration ranges of 0.4e6 to 2.5e6. Dose may be assessed with a manual cell counting process that relies on the exclusion method (Trypan Blue) post TrypLE enzymatic dissociation. Upon staining, live cells are impermeable to Trypan Blue and retain a white appearance, where dead cells are permeable to the dye and turn blue. This method provides reportable values for cell number (total cells/mL or viable cells/mL) and % viability of the pooled cell suspension. In some embodiments, cell count is assessed with two biological replicates, with three technical replicates per biological replicate and two operators executing the count. Final cell concentration may be determined with the following: cells/mL = average manual count per quadrant x final dilution factor x 10e4. In some embodiments, for each count to be valid, each technical replicate ranges from 80-500 cells, and also is within 35% of the mean values of the technical replicates counted for each biological replicate. In some embodiments, the % difference between biological replicates must be less than or equal to 25% to calculate a reportable result for cell concentration and viability. In some embodiments, cell concentration (Viable Cells/mL) may be reported and Viable Cells may be calculated by multiplying the Volume measured in the Gross Weight assessment of the pooled cell suspension by the cell concentration disclosed herein.

$$\begin{matrix}\text{Total viable cells =} \\ \text{Volume of pooled suspension of Drug Substance} \times \text{Viable} \\ \text{Cell Concentration}\end{matrix}$$

### Cell Compositions

In some cases, the SC-β cells of the disclosure share many characteristic features of β cells which are important for normal β cell function. In some embodiments, the SC-β cell exhibits a glucose stimulated insulin secretion (GSIS) response in vitro. In some embodiments, the SC-β cell exhibits a GSIS response in vivo. In some embodiments, the SC-β cell exhibits in vitro and in vivo GSIS responses. In some embodiments, the GSIS responses resemble the GSIS responses of an endogenous mature pancreatic β cell. In some embodiments, the SC-β cell exhibits a GSIS response to at least one glucose challenge. In some embodiments, the SC-β cell exhibits a GSIS response to at least two sequential glucose challenges. In some embodiments, the SC-β cell exhibits a GSIS response to at least three sequential glucose challenges. In some embodiments, the GSIS responses resemble the GSIS response of endogenous human islets to multiple glucose challenges. In some embodiments, the GSIS response is observed immediately upon transplanting the cell into a human or animal. In some embodiments, the GSIS response is observed within approximately 24 hours of transplanting the cell into a human or animal. In some embodiments, the GSIS response is observed within approximately one week of transplanting the cell into a human or animal. In some embodiments, the GSIS response is observed within approximately two weeks of transplanting the cell into a human or animal. In some embodiments, the stimulation index of the cell as characterized by the ratio of insulin secreted in response to high glucose concentrations compared to low glucose concentrations is similar to the stimulation index of an endogenous mature pancreatic β cell. In some embodiments, the SC-β cell exhibits a stimulation index of greater than 1. In some embodiments, the SC-β cell exhibits a stimulation index of greater than or equal to 1. In some embodiments, the SC-β cell exhibits a stimulation index of greater than 1.1. In some embodiments, the SC-β cell exhibits a stimulation index of greater than or equal to 1.1. In some embodiments, the SC-β cell exhibits a stimulation index of greater than 2. In some embodiments, the SC-β cell exhibits a stimulation index of greater than or equal to 1. In some embodiments, the SC-β cell exhibits a stimulation index of at least 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or greater.

In some cases, the cell compositions in the pharmaceutical composition disclosed herein has at least about 30% cells expressing C-peptide and not expressing VMAT1, as measure by flow cytometry. In some cases, the cell composition in the pharmaceutical composition disclosed herein has at least about 35% cells expressing C-peptide and not expressing VMAT1, as measure by flow cytometry. In some cases, the expression of C-peptide and absence of VMAT1 in a cell of the cell compositions suggest that the cell is a SC-β cell. In some cases, the cell composition has at least about 30%, 32%, 33%, 34%, 35%, 36%, 37%, 38%, 39%, 40%, 41%, 42%, 43%, 44%, 45%, 46%, 47%, 48%, 49%, or 50% cells expressing C-peptide and not expressing VMAT1, as measure by flow cytometry. In some cases, the cell composition has about 30%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, or 60% cells expressing C-peptide and not expressing VMAT1, as measure by flow cytometry. In some cases, the cell composition has about 30% to about 60%, about 35% to about 55%, about 40% to about 50% cells expressing C-peptide and not expressing VMAT1, as measure by flow cytometry.

In some cases, the cell composition in the pharmaceutical composition disclosed herein has at most about 35% cells expressing VMAT1, as measure by flow cytometry. In some cases, the cell composition in the pharmaceutical composition disclosed herein has at most about 35% cells expressing VMAT1 and not expressing C-peptide, as measure by flow cytometry. In some cases, the expression of VMAT1 and absence of C-peptide in a cell of the cell compositions suggest that the cell is a SC-EC cell. In some cases, the cell composition has at most about 45%, 40%, 35%, 32%, 31%, 30%, 28%, 25%, 24%, 23%, 22%, 21%, or 20% cells expressing VMAT1 and not expressing C-peptide, as measure by flow cytometry. In some cases, the cell composition has at most about 35%, 32%, 31%, 30%, 28%, 25%, 24%, 23%, 22%, 21%, or 20% cells expressing VMAT1 and not expressing C-peptide, as measure by flow cytometry. In some cases, the cell composition has about 35%, 32%, 31%, 30%, 28%, 25%, 24%, 23%, 22%, 21%, 20%, 19%, 18%, 17%, 16%, or 15% cells expressing VMAT1 and not expressing C-peptide, as measure by flow cytometry. In some cases, the cell composition has about 15% to about 30%, about 16% to 25%, about 17% to about 22%, about 18% to about 20% cells expressing VMAT1 and not expressing C-peptide, as measure by flow cytometry. In some cases, the cell composition has 5-25%, 5-15%, 10-25%, 10-15%, or 15-25% cells expressing VMAT1 and not expressing C-peptide, as measure by flow cytometry.

In some cases, the cell composition includes at least about 3% cells expressing glucagon, as measured by flow cytometry. In some cases, the cell composition includes at least about 5% cells expressing glucagon, as measured by flow cytometry. In some cases, the cell composition includes at least about 10% cells expressing glucagon, as measured by flow cytometry. In some cases, the cell composition includes at least about 20% cells expressing glucagon, as measured by flow cytometry. In some cases, the cell composition includes at least about 15% cells expressing glucagon and not expressing somatostatin, as measured by flow cytometry. In some cases, the expression of glucagon and not expressing somatostatin in a cell of the cell composition suggest that the cell is a SC-α cell. In some cases, the cell composition includes at least about 3%, 5%, 6%, 7%, 8%, 9%, 10%, 11%, 12%, 13%, 14%, 15%, 16%, 17%, 18%, 19%, 20%, 21%, or 22% cells expressing glucagon and not expressing somatostatin, as measured by flow cytometry. In some cases, the cell composition includes at least about 12%, 13%, 14%, 15%, 16%, 17%, 18%, 19%, 20%, 21%, or 22% cells expressing glucagon and not expressing somatostatin, as measured by flow cytometry. In some cases, the cell composition includes about 10% to about 30%, about 12% to about 25%, about 13% to about 22%, about 15% to about 20%, or about 16% to about 18% cells expressing glucagon and not expressing somatostatin, as measured by flow cytometry. In some cases, the cell composition includes about 3%, 5%, 6%, 7%, 8%, 9%, 10%, 12%, 13%, 14%, 15%, 16%, 17%, 18%, 19%, 20%, 21%, or 22% cells expressing glucagon and not expressing somatostatin, as measured by flow cytometry.

In some cases, the cell composition include at least about 1% cells expressing somatostatin and not expressing glucagon, as measured by flow cytometry. In some cases, the cell composition include at least about 2% cells expressing somatostatin and not expressing glucagon, as measured by flow cytometry. In some cases, the cell composition include at least about 3% cells expressing somatostatin and not expressing glucagon, as measured by flow cytometry. In some cases, the cell composition include at least about 4% cells expressing somatostatin and not expressing glucagon, as measured by flow cytometry. In some cases, the expression of glucagon and not expressing somatostatin in a cell of the cell composition suggest that the cell is a SC-δ cell. In some cases, the cell composition include at least about 2%, 3%, 4%, 5%, 6%, 7%, or 8% cells expressing somatostatin and not expressing glucagon, as measured by flow cytometry. In some cases, the cell composition include about 1% to about 9%, about 2% to about 8%, about 3% to about 7%, or about 4% to about 6% cells expressing somatostatin and not expressing glucagon, as measured by flow cytometry. In some cases, the cell composition include about 1%, 2%, 3%, 4%, 5%, 6%, 7%, or 8% cells expressing somatostatin and not expressing glucagon, as measured by flow cytometry.

In some cases, the cell composition has at least about 35% cells expressing C-peptide and not expressing VMAT1, at most about 50% cells expressing VMAT1, at least about 4% cells expressing glucagon and not expressing somatostatin, and at least about 1% cells expressing somatostatin and not expressing glucagon, as measured by flow cytometry. In some cases, the cell composition has at least about 35% cells expressing C-peptide and not expressing VMAT1, at most about 30% cells expressing VMAT1, and at least about 20% cells expressing glucagon, as measured by flow cytometry. In some cases, the cell composition has at least about 35% cells expressing C-peptide and not expressing VMAT1, at most about 30% cells expressing VMAT1, at least about 20% cells expressing glucagon, and at least 4% cells expressing somatostatin and not expressing glucagon, as measured by flow cytometry.

In some cases, the cell composition provided herein includes (a) at least about 35% cells expressing C-peptide and not expressing VMAT1; and (b) at least about 10% cells expressing somatostatin, as measured by flow cytometry. In some cases, there are at least about 15% cells expressing somatostatin in the cell composition, as measured by flow cytometry.

In some cases, provided herein is a composition comprising a population of cells, wherein: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases, provided herein is a composition comprising a population of cells, wherein: (a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1; (b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and/or (c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

In some cases, in the population of cells provided herein, 35-60% of the cells express C-peptide and ISL1 but not VMAT1; 4-25%, of the cells express glucagon but not somatostatin; and 1-10% of the cells express somatostatin but not glucagon. In some cases, less than 40%, less than 35%, less than 30%, less than 25%, less than 20%, less than 18%, less than 15%, less than 12%, or less than 10% of the cells in the population of cells provided herein express VMAT1 but not C-peptide.

In some cases, in the population of cells provided herein, 40-60% of the cells express C-peptide and ISL1 but not VMAT1; 10-25% of the cells express glucagon but not somatostatin; and 4-10% of the cells express somatostatin but not glucagon. In some cases, less than 25%, less than 20%, less than 18%, less than 15%, less than 12%, or less than 10% of the cells in the population of cells provided herein express VMAT1 but not C-peptide.

In some embodiments, any of the pharmaceutical compositions disclosed herein comprises no less than 50%, 40%, 30%, or 20% NKX6.1⁺/ISL1⁺ cells (e.g., as determined by flow cytometry). In some embodiments, no less than 30% of the cells in the composition are NKX6.1-positive, ISL1-positive cells, no less than 25% of the cells in the composition are NKX6.1-negative, ISL1-positive cells, less than 12% of the cells in the composition are NKX6.1-negative, ISL1-negative cells or between 9-25% of the cells in the composition are NKX6.1-positive, ISL1-negative cells (e.g., as determined by flow cytometry). In some embodiments, no less than 40%, 35%, 30%, 26%, 25%, or 20% of the cells in the pharmaceutical composition are NKX6.1⁻/ISL1⁺ cells (e.g., as determined by flow cytometry). In some embodiments, no less than 26% of the cells in the pharmaceutical composition are NKX6.1⁻/ISL1⁺ cells (e.g., as determined by flow cytometry). In some embodiments, between 5-25%, 5-40%, 5-35%, or 8-20% of the cells in the pharmaceutical composition are NKX6.1⁻/ISL1⁺ cells (e.g., as determined by flow cytometry). In some embodiments, no more than 50%, 45%, 40%, 35%, 30%, or 25% of the cells in the pharmaceutical composition are NKX6.1⁺/ISL1⁻ cells (e.g., as determined by flow cytometry). In some embodiments, no more than 50% of the cells in the pharmaceutical composition are NKX6.1⁺/ISL1⁻ cells (e.g., as determined by flow cytometry).

In some embodiments, less than 12% of the cells (e.g., about 11%, about 10%, about 9%, about 8%, about 7%, about 6%, about 5%, about 4%, about 3%, about 2%, about 1%, or less) in the population are NKX6.1-negative, ISL1-negative cells. In some embodiments, less than 10%, less than 8%, less than 6%, less than 4%, or 1%-11%, 2%-10%, 2%-12%, 4%-12%, 6%-12%,8%-12%, 2%-8%, 4%-8%, 3%-6% or 3%-5% of the cells in the population are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the population are NKX6.1-negative, ISL1-positive cells. In some embodiments, at least 15%, at least 20%, at least 25%, at least 30%, at least 35%, at least 40%, at least 45%, at least 50%, at least 55%, at least 60%, or 15%-60%, 15%-45%,15%-30%, 30%-60%, 30%-45%, 45%-60% of the cells in the population are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the population are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the population are NKX6.1-negative, ISL1-positive cells and less than 12% (e.g., 2%-12%, 4%-12%, 6%-12%, 8%-12%, 2%-8%, 4%-8%, 3%-6% or 3%-5%) of the cells in the population are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the population are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the population are ISL1-positive cells. In some embodiments, at least 74%, at least 75%, at least 80%, at least 85%, at least 90%, about 85-95%, or about 90-95% of the cells in the population are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the population are ISL1-positive cells.

In some embodiments, a population of in vitro differentiated cells described herein comprises more NKX6.1-negative, ISL1-positive cells than NKX6.1-positive, ISL1-positive cells. In some embodiments, at least 40% of the cells in the population are NKX6.1-negative, ISL1-positive cells. In some embodiments, at least 45%, at least 50%, about 40-50%, about 45-55%, or about 50-55% of the cells in the population are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the population are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, at least 20% (e.g., at least 20%, at least 30%, at least 40%, at least 50%, at least 50%, at least 60% or more) of the cells in the composition are ISL1-positive and NKX6.1-positive. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-positive. In some embodiments, about 20%, 25%, 30%, 35%, 40%, 45%, 50%, 55%, 60% or more of the cells in the composition are ISL1-positive and NKX6.1-positive.

In some embodiments, at least 20% (e.g., at least 20%, at least 30%, at least 40%, at least 50%, at least 50%, at least 60% or more) of the cells in the composition are ISL1-positive and NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-negative. In some embodiments, about 20%, 25%, 30%, 35%, 40%, 45%, 50%, 55%, 60% or more of the cells in the composition are ISL1-positive and NKX6.1-negative.

In some embodiments, a population of in vitro differentiated cells described herein comprises up to 20% (e.g., up to 20%, up to 30%, up to 40% or up to 50%) of NXK6.1-positive, ISL1-positive cells. In some embodiments, a population of in vitro differentiated cells described herein comprises about 20%-50%, 20%-40%, 20%-30%, 30%-50%, 30%-40%, or 40%-50% of NXK6.1-positive, ISL1-positive cells. In some embodiments, a population of in vitro differentiated cells described herein comprises about 20%-50%, 20%-40%, 20%-30%, 30%-50%, 30%-40%, or 40%-50% of NXK6.1-positive, ISL1-positive cells.

In some embodiments, the NKX6.1-positive, ISL1-positive cells also express PDX1.

In some embodiments, the disclosure provides for a composition comprising a plurality of cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 1-12%, 1-10%, 1-8%, 1-6%, 1-4%, 3-5%, 1-2%, 2-12%, 2-10%, 2-8%, 2-6%, 2-4%, 4-12%, 4-10%, 4-8%, 4-6%, 6-12%, 6-10%, 6-8%, 8-12%, 8-10%, or 10-12% of the cells in the composition are NKX6.1-negative, ISL1-negative cells. In some embodiments, the disclosure provides for a composition comprising a plurality of cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 2-12% of the cells in the composition are NKX6.1-negative, ISL1-negative cells. In some embodiments, between 3-25%, 3-20%, 3-15%, 3-10%, 3-5%, 5-25%, 5-20%, 5-15%, 5-10%, 10-25%, 10-20%, 10-15%, 15-25%, 15-20% or 20-25% of the cells in the composition are NKX6.1-positive, ISL1-negative cells.

In some embodiments, the disclosure provides for a composition comprising a plurality of cells (e.g., a composition comprising a cluster of cells or multiple clusters of cells); wherein at least 30% of the cells in the composition are NKX6.1-positive, ISL1-positive cells; wherein at least 25% of the cells in the composition are NKX6.1-negative, ISL1-positive cells; and wherein between 9-25% of the cells in the composition are NKX6.1-positive, ISL1-negative cells. In some embodiments, the disclosure provides for a composition comprising a plurality of cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 9-30%, 9-25%, 9-20%, 9-15%, 9-12%, 12-30%, 12-25%, 12-20%, 12-15%, 15-30%, 15-25%, 15-20%, 20-30%, 20-25% or 25-30% of the cells in the composition are NKX6.1-positive ISL-negative cells. In some embodiments, 1-12%, 1-10%, 1-8%, 1-6%, 1-4%, 3-5%, 1-2%, 2-12%, 2-10%, 2-8%, 2-6%, 2-4%, 4-12%, 4-10%, 4-8%, 4-6%, 6-12%, 6-10%, 6-8%, 8-12%, 8-10%, or 10-12% of the cells in the composition are NKX6.1-negative, ISL1-negative cells. In some embodiments, the disclosure provides for a composition comprising a plurality of cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 9-25% of the cells in the composition are NKX6.1-positive, ISL1-negative cells.

In some embodiments, less than 12% of the cells (e.g., about 11%, about 10%, about 9%, about 8%, about 7%, about 6%, about 5%, about 4%, about 3%, about 2%, about 1%, or less) in the composition are NKX6.1-negative, ISL1-negative cells. In some embodiments, less than 10%, less than 8%, less than 6%, less than 4%, 1%-11%, 2%-10%, 2%-12%, 4%-12%, 6%-12%, 8%-12%, 2%-8%, 4%-8%, 3%-6% or 3%-5% of the cells in the composition are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the composition are NKX6.1-negative, ISL1-positive cells. In some embodiments, at least 15%, at least 20%, at least 25%, at least 30%, at least 35%, at least 40%, at least 45%, at least 50%, at least 55%, at least 60%, 15%-60%, 15%-45%,15%-30%, 30%-60%, 30%-45%, 45%-60% of the cells in the composition are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the composition are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the composition are NKX6.1-negative, ISL1-positive cells and less than 12% (e.g., 2%-12%, 4%-12%, 6%-12%, 8%-12%, 2%-8%, 4%-8%, 3%-6% or 3%-5%) of the cells in the composition are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the composition are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the composition are ISL1-positive cells. In some embodiments, at least 74%, at least 75%, at least 80%, at least 85%, at least 90%, about 85-95%, or about 90-95% of the cells in the composition are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the composition are ISL1-positive cells.

In some embodiments, the composition comprises more NKX6.1-positive, ISL1-positive cells that NKX6.1-negative, ISL1-positive cells. In some embodiments, at least 40% of the cells in the composition are NKX6.1-negative, ISL1-positive cells. In some embodiments, at least 45%, at least 50%, about 40-50%, about 45-55%, or about 50-55% of the cells in the composition are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the composition are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, the composition comprises at least 20% (e.g., at least 20%, 30%, 40%, 50% or 60%) of NXK6.1-positive, ISL1-positive cells. In some embodiments, the composition comprises about 20%-50%, 20%-40%, 20%-30%, 30%-50%, 30%-40%, 40%-50%, 40%-60%, or 50-60% of NXK6.1-positive, ISL1-positive cells. In some embodiments, the composition comprises about 20%-50%, 20%-40%, 20%-30%, 30%-50%, 30%-40%, or 40%-50% of NXK6.1-positive, ISL1-positive cells.

In some embodiments, the composition comprises less than 25% (e.g., less than 25%, less than 20%, less than 15%, less than 10%, less than 5%, or less) of NKX6.1-positive, ISL1-negative cells. In some embodiments, the composition comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, the compositioncomprises about 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, the composition comprises about 2%, 4%, 6%, 8%, or 10% of NKX6.1-positive, ISL1-negative cells.

In some embodiments, the composition comprises less than 10% SOX9-positive cells. In some embodiments, the composition comprises less than 10%, 9%, 8%, 7%, 6%, 5%, 4%, 3%, 2% or 1% SOX9-positive cells. In some embodiments, the composition comprises 0.1-10%, 0.1-7%, 0.1-3%, 0.1-1%, 0.5-10%, 0.5-7%, 0.5-3%, 0.5-1%, 1-10%, 1-5%, 1-3%, 3-10%, 3-5%, or 5-10% SOX9-positive cells.

In some embodiments, the composition comprises less than 5% Ki67-positive cells. In some embodiments, the composition comprises less than 5%, 4%, 3%, 2% or 1% Ki67-positive cells. In some embodiments, the composition comprises 0.01-0.1%, 0.1-5%, 0.1-3%, 0.1-1%, 0.5-5%, 0.5-3%, 0.5-1%, 1-5%, 1-3%, or 1-2% Ki67-positive cells.

In some embodiments, at least 80%, 85%, 90%, 91%, 92%, 93%, 94%, 95%, 96%, 97%, 98%, or 99% of the cells in the composition are CHGA-positive cells. In some embodiments, 80-100%, 85-100%, 90-100%, 90-99%, 90-98%, 95-99%, or 95-99% of the cells in the composition are CHGA-positive cells.

In some embodiments, the percentage of cells expressing a marker provided herein is measured by flow cytometry. The skilled worker is aware of representative methods for testing whether a cell or collection of cells is positive or negative for expression of a specific gene marker (e.g., NKX6.1, ISL1, INS, GCG, somatostatin, chromogranin A, SOX9, C-peptide, Ki67) by flow cytometry. In some embodiments, a cell is considered positive for expression of a particular gene (e.g., NKX6.1, ISL1, INS, GCG, somatostatin, chromogranin A, SOX9, C-peptide, Ki67) based on median fluorescence intensity (rMFI). As used herein, the term “rMFI” or relative median fluorescence intensity is the ratio between the fluorescence intensity measured by use of an antibody to a specific target (e.g., NKX6.1, ISL1, INS, GCG, somatostatin, chromogranin A, SOX9, C-peptide, Ki67) versus the intensity obtained from a control antibody (isotype control). In some embodiments, an anti-(human) NKX6.1, ISL1, INS, GCG, somatostatin, chromogranin A, or SOX9 antibody is used. Examples of suitable antibodies for use in flow cytometry are any of the antibodies disclosed in Table 1. An example of a suitable flow cytometer is the Accuri 6 flow cytometer. In some embodiments, the target-expressing cells (e.g., cells expressing NKX6.1 and/or ISL1), if tested, exhibit a target relative medium fluorescence intensity (rMFI) of at least 6, 6.5, 7, 8, 9 or 10 as measured by flow cytometry. In another embodiment, said rMFI is between 6.5 and 15, between 6.5 and 14, between 6.5 and 13, between 6.5 and 13, between 6.5 and 12, or between 6.5 and 10.

TABLE 1

| | | | | | |
|---|---|---|---|---|---|
| Primary Antibody | Company | Cat# | Primary Antibody Species | Secondary Antibody Species | Cat# |
| NKX6.1 | DSHB | FSSA12 | Mouse | anti-Mouse 488 | A21202 |
| IsL1 | abcam | ab178400 | Rabbit | anti-Rabbit-647 | A31573 |
| SST | Santa Cruz Biotechnolog y | SC-55565 AF647 | Anti-Somatostatin Antibody (G-10) Alexa Fluor® 647 | AlexaFluor® 647 | - |
| Glu | R&D | IC 1249G | Human/Mouse Glucagon Alexa Fluor® 488-conjugated | AlexaFluor®4 88 | - |
| Sox9 | Epitomics | AC-0284RU OC | Rabbit | anti-Rabbit-647 | A31573 |
| Ki67 | Thermo Fisher | | Ki-67 Monoclonal Antibody (SolA15), PE | PE | - |
| C-pep | DSHB | GN-1D4-S | Rat | anti-Rat-488 | A21208 |
| CHGA | abcam | ab15160 | Rabbit | anti-Rabbit-647 | A31573 |

In some embodiments, the percentage of cells expressing a marker provided herein is measured by qRT-PCR. In some embodiments, the percentage of cells expressing a marker provided herein is measured by single cell RNA sequencing analysis. The skilled worker is aware of methods for testing whether a cell or collection of cells is positive for expression of a specific gene marker (e.g., NKX6.1, ISL1, INS, GCG, ARX, or ghrelin) by single cell RNA sequencing analysis.

In some embodiments, a population of in vitro differentiated cells described herein comprises less than 25% (e.g., less than 25%, less than 20%, less than 15%, less than 10%, less than 5%, or less) of NKX6.1-positive, ISL1-negative cells. In some embodiments, a population of in vitro differentiated cells described herein comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, a population of in vitro differentiated cells described herein comprises about 2%-10%, 2%-8%, 2%-6%, 2%-4%, 4%-10%, 4%-8%,4%-6%, 6%-10%, 6%-8%, or 8%-10% of NKX6.1-positive, ISL1-negative cells. In some embodiments, a population of in vitro differentiated cells described herein comprises about 2%, 4%, 6%, 8%, or 10% of NKX6.1-positive, ISL1-negative cells.

In some embodiments, the disclosure provides for a pharmaceutical composition comprising NKX6.1-positive, ISL1-positive cells that express lower levels of MAFA than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express higher levels of MAFB than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express higher levels of SIX2, HOPX, IAPP and/or UCN3 than NKX6.1-positive, ISL1-positive cells from the pancreas of a healthy control adult subject or from a cadaveric pancreas. In some embodiments, the pharmaceutical composition comprises NKX6.1-positive, ISL1-positive cells that do not express MAFA. In some embodiments, the pharmaceutical composition comprises NKX6.1-positive, ISL1-positive cells that express MAFB. In some embodiments, the pharmaceutical composition comprises cells that are genetically modified (e.g., using a gene editing technology such as CRISPR). In some embodiments, the pharmaceutical composition comprises NKX6.1-positive, ISL1-positive cells that express lower levels of beta-2 microglobulin, CIITA, HLA-A, HLA-B, HLA-C, HLA-DP, HLA-DQ, and HLADR than NKX6.1-positive, ISL1-positive cells from the pancreas of a healthy control adult subject or from a cadaveric pancreas. In some embodiments, the pharmaceutical composition comprises NKX6.1-positive, ISL1-positive cells that express increased levels of CD47, PDL1, HLA-G, CD46, CD55, CD59 and CTLA than NKX6.1-positive, ISL1-positive cells from the pancreas of a healthy control adult subject or from a cadaveric pancreas. In some embodiments, any of the cell markers disclosed herein (e.g., NKX6.1, PDX1, MAFA, MAFB, SIX2, HOPX, IAPP and/or UCN3) are detected by flow cytometry.

In some embodiments, the pharmaceutical composition is derived from stem cells in vitro. In some embodiments, the stem cells are genetically modified (e.g., using a gene editing technology such as CRISPR). In some embodiments, the stem cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the stem cells have increased expression of CD47, PDL1, HLA-G, CD46, CD55, CD59 and CTLA, relative to stem cells that are not genetically modified. In some embodiments, any of the cell markers disclosed herein (e.g., NKX6.1, PDX1, MAFA, MAFB, SIX2, HOPX, IAPP and/or UCN3) are detected by flow cytometry.

In some cases, cell populations or cell clusters disclosed herein are unsorted, e.g., isolated cell populations or cell clusters that have not been through cell sorting process. In some embodiments, the cell cluster disclosed herein can refer to a cell cluster formed by self-aggregation of cells cultured in a given environment, for instance, in a 3D suspension culture. Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells’ optical properties (e.g., emission wave-length upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in the cells. The GFP signal can thus be a marker of a pancreatic β cell. In some cases, cell sorting as described herein can comprise subjecting cells to magnetic-activated sorting process, where magnetic antibody or other ligand is used to label cells of different types, and the differences in magnetic properties can be used for cell sorting.

The percentage of cells expressing one or more particular markers, like PDX1, NKX6.1, insulin, NGN3, or CHGA, described herein can be the percentage value detected using techniques like flow cytometry assay. In some cases, during a flow cytometry assay, cell population or cell cluster discussed herein are dispersed into single-cell suspension by incubation in digesting enzyme like trypsin or TrypLE™ Express. Dispersed cell can be washed in suitable buffer like PBS, centrifuged and then re-suspended in fixation buffer like 4% PFA. Incubation with primary antibodies against the cell markers of interest can then be conducted, which can be followed by incubation with the secondary antibodies. After antibody incubation, the cells can be washed and the subject to segregation by flow cytometry. Techniques other than flow cytometry can also be used to characterize the cells described herein, e.g., determine the cell percentages. Non-limiting examples of cell characterization methods include gene sequencing, microscopic techniques (fluorescence microscopy, atomic force microscopy), karyotyping, isoenzyme analysis, DNA properties, viral susceptibility.

In some embodiments, any of the cells disclosed herein comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, any of the cells disclosed herein (e.g., any of the SC-β cells or cells in any of the clusters disclosed herein) comprise a genomic disruption in at least one gene sequence, wherein said disruption reduces or eliminates expression of a protein encoded by said gene sequence. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLAB, but do not comprise a genomic disruption in the gene encoding HLA-C. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen- related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to cells that are not genetically modified. In some embodiments, the cells have increased expression of CD47, PDL1, HLA-G, CD46, CD55, CD59 and CTLA, relative to cells that are not genetically modified. In particular embodiments, the pancreatic islet cells disclosed herein (e.g., the SC-beta cells) have increased expression of PDL1 as compared to endogenous pancreatic islet cells from a healthy control subject. In particular embodiments, the pancreatic islet cells disclosed herein (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology.

In some embodiments, any of the cells disclosed herein comprise a genomic disruption in at least one gene sequence, wherein said disruption reduces or eliminates expression of a protein encoded by said gene sequence (e.g., by knocking out one or both functional copies of a gene in a subject). In some embodiments, said at least one gene sequence is the ABO sequence, such that the disruption results in the cell being blood type O. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLA-B, but do not comprise a genomic disruption in the gene encoding HLA-C. In some embodiments, the cells comprise a genomic disruption in the gene encoding CXCL10. In some embodiments, the cells comprise a genomic disruption in the gene encoding renalase. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen- related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the cells have increased expression of CD47, PDL1, HLA-G, CD46, CD55, CD59, CTLA, PDL2, HLA-C, HLA-E, HLA-G, C1-inhibitor, IL-35, DUX4, IDO1, IL10, CCL21, CCL22, CD16, CD52, H2-M3, CD200, FASLG, MFGE8, and/or SERPINB9 relative to cells that are not genetically modified. In particular embodiments, the pancreatic islet cells disclosed herein (e.g., the SC-beta cells) have increased expression of PDL1 as compared to endogenous pancreatic islet cells from a healthy control subject. In particular embodiments, the pancreatic islet cells disclosed herein (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology. In some embodiments, any of the isolated cells (e.g., a stem cell or a NKX6.1-positive, ISL1-positive cell) described herein comprises a disruption (e.g., deletion, insertion, translocation, inversion, or substitution of one or more nucleotides) in any one or more of the genes encoding: B2M, CIITA, CXCL10, renalase, HLA-A, HLA-B, HLA-C, RFX-ANK, NFY-A, NLRC5, RFX5, RFX-AP, HLA-G, HLA-E, NFY-B, PD-L1, NFY-C, IRF1, TAPI, GITR, 4-1BB, CD28, B7-1, CD47, B7-2, 0X40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, IDO1, TRAC, TRB, NFY-A, CCR5, F3, CD142, MICA, MICB, LRP1, HMGB1, ABO, RHD, FUT1, KDM5D, PDGFRa, OLIG2, and/or GFAP. In some embodiments, disruption of a gene results in an at least 10%, 20%, 30%, 40%, 50%, 60%, 70%, 80%, 90% or 100% decrease in expression of the gene as compared to the expression of the gene in the same type of cell without the disruption. In some embodiments, the gene is disrupted using CRISPR/Cas, piggybac transposon, TALEN, and/or zinc finger technology.

In some embodiments, a cell (e.g., an isolated stem cell or a NKX6.1-positive, ISL1-positive cell) described herein is negative for A antigen and negative for B antigen. In some embodiments, the cell described herein is negative for A antigen. In some embodiments, the cell described herein is negative for B antigen. In some embodiments, a cell (e.g., an isolated stem cell or a NKX6.1-positive, ISL1-positive cell) described herein is negative for Rh antigen. In some embodiments, a cell (e.g., an isolated stem cell or a NKX6.1-positive, ISL1-positive cell) described herein is negative for A antigen, negative for B antigen, and negative for Rh antigen. An “A antigen,” as used herein, refers to a histo-blood group antigen produced by 3α-N-acetylgalactosaminyltransferase and expressed as a cell-surface antigen. A “B antigen,” as used herein, refers to a histo-blood group antigen produced by 3α-galactosaminyltransferase and expressed as a cell-surface antigen. In some embodiments, the cell comprises a disruption in the ABO gene. In some embodiments, the cell comprises a disruption in the ABO gene such that the cell has reduced or absent levels of A and B antigens. In some embodiments, the cell comprises a disruption in the FUT1 gene. In some embodiments, the cell comprises a disruption in the FUT1 gene such that Galactoside 2-alpha-L-fucosyltransferase 1 expression is reduced or absent. An “Rh antigen,” as used herein, refers to a highly immunogenic antigen encoded by two highly polymorphic genes, RHD and RHCE. Rh antigen proteins are transmembrane proteins. In some embodiments, the cell comprises a disruption in the RHAG gene. In some embodiments, the cell comprises a disruption in the RHAG gene such that the cell has reduced or absent levels of Rh-associated glycoprotein. In some embodiments, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

In some embodiments, any of the cells disclosed herein (e.g., any of the SC-islet cells disclosed herein) comprises a “safety switch.” In some embodiments, the safety switches are nucleic acid constructs encoding a switch protein that inducibly causes cell death or stops cell proliferation. In some embodiments, the safety switch is inserted at a defined, specific target locus (e.g., a safe harbor locus) in the genome of an engineered cell, usually at both alleles of the target locus. In some embodiments, the target locus is a safe harbor locus, such as ActB or CLYBL. In some embodiments, the target locus is a gene targeted for disruption (e.g., B2M or CIITA). In some embodiments, the switch protein is activated by contacting with an effective dose of a clinically acceptable orthologous small molecule. In some embodiments, when activated, the safety switch causes the cell to stop proliferation, in some embodiments by activating apoptosis of the cell. In some embodiments, the switch protein comprises herpes-simplex-thymidine-kinase. In some embodiments the switch protein comprises a human caspase protein, e.g. caspase 1, caspase 2, caspase 3, caspase 4, caspase 5, caspase 6, caspase 7, caspase 8, caspase 9, caspase 10, caspase 14, etc. In certain embodiments the protein is human caspase 9. In some embodiments, the caspase protein is fused to a sequence that provides for chemically induced dimerization (CID), in which dimerization occurs only in the presence of the orthologous activating agent. One or more CID domains may be fused to the caspase protein, e.g. two different CID domains may be fused to the caspase protein. In some embodiments the CID domain is a dimerization domain of FKBP or FRB (FKBP-rapamycin-binding) domain of mTOR, which are activated with rapamycin analogs. In some embodiments, the safety switch is any of the safety switches described in WO2021173449 and Jones et al., 2014, Frontiers in Pharmacology, 5(254):1-8, each of which is incorporated herein in its entirety.

In some embodiments, in the pharmaceutical composition disclosed herein, at least a portion of the cells in the population of cells are present in plurality of cell clusters. In some cases, the cell clusters are about 50 µm to about 500 µm, about 50 µm to about 400 µm, about 50 µm to about 300 µm, about 60 µm to about 400 µm, about 60 µm to about 300 µm, about 60 µm to about 250 µm, about 75 µm to about 400 µm, about 75 µm to about 300 µm, about 75 µm to about 250 µm, about 125 µm to about 225 µm, about 130 µm to about 160 µm, about 170 µm to about 225 µm, about 140 µm to about 200 µm, about 140 µm to about 170 µm, about 160 µm to about 220 µm, about 170 µm to about 215 µm, or about 170 µm to about 200 µm in diameter. In some cases, in the pharmaceutical compositions disclosed herein, the population of cells are present as a single cell suspsension. In some embodiments, in the pharmaceutical compositions disclosed herein, at least 50%, at least 60%, at least 70%, at least 75%, at least 80%, at least 85%, at least 90%, at least 92%, at least 94%, at least 95%, at least 96%, at least 98%, or at least 99% of the cells are present in cell clusters. In some embodiments, in the pharmaceutical compositions disclosed herein, substantially all of the cells are present in cell clusters, e.g., at least 99.5%, at least 99.6%, at least 99.7%, at least 99.8%, at least 99.9%, at least 99.9%, at least 99.99%, at least 99.999%, or at least 99.9999% of the cells.

### Pharmaceutically Acceptable Excipients and/or Carriers

In some aspects, the pharmaceutical compositions disclosed herein includes a population of cells in a liquid suspension. The liquid suspension can contain an aqueous solution that comprises pharmaceutically acceptable excipient(s) and/or carrier(s). For instanace, the pharmaceutical compositions can further comprise a physiologically compatible solution including, for example, phosphate-buffered saline.

In some cases, the present disclosure provides pharmaceutical compositions that can utilize non-native pancreatic β cell populations and cell components and products in various methods for treatment of a disease (e.g., diabetes). Certain cases encompass pharmaceutical compositions comprising live cells (e.g., non-native pancreatic β cells alone or admixed with other cell types). Other cases encompass pharmaceutical compositions comprising non-native pancreatic β cell components (e.g., cell lysates, soluble cell fractions, conditioned medium, ECM, or components of any of the foregoing) or products (e.g., trophic and other biological factors produced by non-native pancreatic β cells or through genetic modification, conditioned medium from non-native pancreatic β cell culture). In either case, the pharmaceutical composition may further comprise other active agents, such as anti-inflammatory agents, exogenous small molecule agonists, exogenous small molecule antagonists, anti-apoptotic agents, antioxidants, and/or growth factors known to a person having skill in the art.

Pharmaceutical compositions of the present disclosure can comprise non-native pancreatic β cell, or components or products thereof, formulated with a pharmaceutically acceptable carrier (e.g. a medium or an excipient). The term pharmaceutically acceptable carrier (or medium or excipient), which may be used interchangeably with the term biologically compatible carrier or medium, can refer to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer’s solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as a liquid suspension.

Pharmaceutical compositions may comprise auxiliary components as would be familiar to a person having skill in the art. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other anti-oxidant agents suitable for renal administration, e.g. ascorbic acid and its salts or sulfite or sodium metabisulfite may also be employed.

A buffering agent may be used to maintain the pH of formulations in the range of about 4.0 to about 8.0; so as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Pharmaceutical compositions comprising cells, cell components or cell products may be delivered to the liver of a patient in one or more of several methods of delivery known in the art. In some cases, the compositions are delivered to the liver. In another embodiment, the compositions may be delivered to the liver via intra-portal injection.

In some embodiments, any of the cell compositions disclosed herein (e.g., a composition comprising in vitro differentiated islet cells) further comprises a liquid solution (or a medium). In some embodiments, the liquid solution comprises a sugar. In some embodiments, the sugar is sucrose or glucose. In some embodiments, the liquid solution comprises the sugar at a concentration of between about 0.05% and about 1.5%. In some embodiments, the liquid solution is a CMRL medium. In some embodiments, the composition comprises HypoThermosol® FRS Preservation Media. In some cases, the liquid solution is serum free. In some cases, the liquid solution that the cells are suspended in is free of proteins. In some cases, the liquid solution that the cells are suspended in is free of animal components. In some cases, the liquid solution that the cells are suspended in is free of proteins and animal components. In some cases, the liquid solution has physiological osmolality. In some cases, the liquid solution is pH buffered.

In some instances, pharmaceutical compositions of the stem cell derived islet cells are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration. In some embodiments, any of the pharmaceutical compositions disclosed herein is administered to a subject by infusion via the hepatic portal vein.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington’s Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, a composition of the present disclosure can comprise the stem cell derived islet cells, in an amount that is effective to treat or prevent e.g., diabetes. A pharmaceutical composition can comprise the stem cell derived islet cells as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions can comprise auxiliary components as would be familiar to a person having skill in the art. For example, they can contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, β-mercaptoethylamine, glutathione and similar species, although other anti-oxidant agents suitable for renal administration, e.g. ascorbic acid and its salts or sulfite or sodium metabisulfite may also be employed.

A buffering agent can be used to maintain the pH of formulations in the range of about 4.0 to about 8.0; soas to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and an anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween ® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner’s sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, any of the compositions disclosed herein are encapsulated in any of the devices disclosed herein.

### Stem Cells and Reprogramming

Provided herein is use of stem cells for producing SC-β cells (e.g., mature pancreatic β cells or β-like cells) or precursors thereof. In an embodiment, germ cells may be used in place of, or with, the stem cells to provide at least one SC-β cell, using similar protocols as described in U.S. Pat. Application Publication No. US20150240212 and US20150218522, each of which is herein incorporated by reference in its entirety. Suitable germ cells can be prepared, for example, from primordial germ cells present in human fetal material taken about 8-11 weeks after the last menstrual period. Illustrative germ cell preparation methods are described, for example, in Shamblott et al., Proc. Natl. Acad. Sci. USA 95:13726, 1998 and U.S. Pat. No. 6,090,622.

Provided herein are compositions and methods of generating SC-islet cells (e.g., SC-β cells), as well as SC-α cells, and/or SC-δ cells.

Generally, the at least one SC-islet cell or precursor thereof, e.g., pancreatic progenitors produced according to the methods disclosed herein can comprise a mixture or combination of different cells.

The at least one pancreatic α, β and/or δ cell or precursor thereof can be produced according to any suitable culturing protocol to differentiate a stem cell or pluripotent cell to a desired stage of differentiation. In some embodiments, the at least one pancreatic α, β and/or δ cell or the precursor thereof are produced by culturing at least one pluripotent cell for a period of time and under conditions suitable for the at least one pluripotent cell to differentiate into the at least one pancreatic α, β and/or δ cell or the precursor thereof.

In some embodiments, the at least one pancreatic α, β and/or δ cell or precursor thereof is a substantially pure population of pancreatic α, β and/or δ cells or precursors thereof. In some embodiments, a population of pancreatic α, β and/or δ cells or precursors thereof comprises a mixture of pluripotent cells or differentiated cells. In some embodiments, a population of pancreatic α, β and/or δ cells or precursors thereof are substantially free or devoid of embryonic stem cells or pluripotent cells or iPS cells.

In some embodiments, a somatic cell, e.g., fibroblast can be isolated from a subject, for example as a tissue biopsy, such as, for example, a skin biopsy, and reprogrammed into an induced pluripotent stem cell for further differentiation to produce the at least one pancreatic α, β and/or δ cell or precursor thereof for use in the compositions and methods described herein. In some embodiments, a somatic cell, e.g., fibroblast is maintained in culture by methods known by one of ordinary skill in the art, and in some embodiments, propagated prior to being converted into pancreatic α, β and/or δ cells by the methods as disclosed herein.

In some embodiments, the at least one pancreatic α, β and/or δ cell or precursor thereof are maintained in culture by methods known by one of ordinary skills in the art, and in some embodiments, propagated prior to being converted into pancreatic α, β and/or δ cells by the methods as disclosed herein.

Further, at least one pancreatic α, β and/or δ cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian at least one pancreatic α, β and/or δ cell or precursor thereof but it should be understood that all of the methods described herein can be readily applied to other cell types of at least one pancreatic α, β and/or δ cell or precursor thereof. In some embodiments, the at least one pancreatic α, β and/or δ cell or precursor thereof is derived from a human individual.

### Stem Cells

Embodiments of the present disclosure are related to use of stem cells for generation of pancreatic α, β and/or δ cells or precursors thereof. The term “stem cell” as used herein can refer to a cell (e.g., vertebrate stem cell) that has the ability both to self-renew and to generate a differentiated cell type (Morrison et al., (1997) Cell 88:287-298). In the context of cell ontogeny, the adjective “differentiated”, or “differentiating” is a relative term. A “differentiated cell” can be a cell that has progressed further down the developmental pathway than the cell it is being compared with. Thus, pluripotent stem cells can differentiate into lineage-restricted progenitor cells (e.g., definitive endoderm cells), which in turn can differentiate into cells that are further restricted (e.g., pancreatic progenitors), which can differentiate into end-stage cells (e.g., terminally differentiated cells, e.g., beta-cells, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest, e.g., that can be used in the method provided herein, can include pluripotent stem cells (PSCs). The term “pluripotent stem cell” or “PSC” as used herein can refer to a stem cell capable of producing all cell types of the organism. Therefore, a PSC can give rise to cells of all germ layers of the organism (e.g., the endoderm, mesoderm, and ectoderm of a vertebrate). Pluripotent cells can be capable of forming teratomas and of contributing to ectoderm, mesoderm, or endoderm tissues in a living organism. Pluripotent stem cells of plants can be capable of giving rise to all cell types of the plant (e.g., cells of the root, stem, leaves, etc.).

Embodiments of the present disclosure are related to use of PSCs for generation of pancreatic α, β and/or δ cells or precursors thereof. PSCs of animals can be derived in a number of different ways. For example, embryonic stem cells (ESCs) can be derived from the inner cell mass of an embryo (Thomson et. al, Science. 1998 Nov. 6; 282(5391):1145-7) whereas induced pluripotent stem cells (iPSCs) can be derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC can refer to pluripotent stem cells regardless of their derivation, the term PSC can encompass the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

Embodiments of the present disclosure are related to use of ESCs for generation of pancreatic β cells or precursors thereof. By “embryonic stem cell” (ESC) can be meant a PSC that is isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs can grow as flat colonies with large nucleo-cytoplasmic ratios, defined borders and prominent nucleoli. In addition, ESCs can express SSEA-3, SSEA-4, TRA-1-60, TRA-1-81, and Alkaline Phosphatase, but not SSEA-1. Examples of methods of generating and characterizing ESCs can be found in, for example, U.S. Pat. No. 7,029,913, U.S. Pat. No. 5,843,780, and U.S. Pat. No. 6,200,806, each of which is incorporated herein by its entirety. Methods for proliferating hESCs in the undifferentiated form are described in WO 99/20741, WO 01/51616, and WO 03/020920, each of which is incorporated herein by its entirety.

By “embryonic germ stem cell” (EGSC) or “embryonic germ cell” or “EG cell”, it can be meant a PSC that is derived from germ cells and/or germ cell progenitors, e.g. primordial germ cells, e.g. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

Embodiments of the present disclosure are related to use of iPSCs for generation of pancreatic α, β and/or δ cells or precursors thereof. By “induced pluripotent stem cell” or “iPSC”, it can be meant a PSC that is derived from a cell that is not a PSC (e.g., from a cell this is differentiated relative to a PSC). iPSCs can be derived from multiple different cell types, including terminally differentiated cells. iPSCs can have an ES cell-like morphology, growing as flat colonies with large nucleo-cytoplasmic ratios, defined borders and prominent nuclei. In addition, iPSCs can express one or more key pluripotency markers known by one of ordinary skill in the art, including but not limited to Alkaline Phosphatase, SSEA3, SSEA4, Sox2, Oct¾, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Pat. Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

In some embodiments, the population of cells is derived from stem cells in vitro. In some embodiments, the stem cells are genetically modified. In some embodiments, the stem cells have reduced expression of one or more of beta-2 microglobulin, CIITA, HLA-A, HLA-B, HLA-C, HLA-DP, HLA-DQ, and HLA-DR, relative to stem cells that are not genetically modified. In some embodiments, the stem cells have increased expression of one or more ofCD47, PDL1, HLA-G, CD46, CD55, CD59 and/or CTLA, relative to stem cells that are not genetically modified.

Embodiments of the present disclosure are related to use of somatic cells for generation of pancreatic α, β and/or δ cells or precursors thereof. By “somatic cell”, it can be meant any cell in an organism that, in the absence of experimental manipulation, does not ordinarily give rise to all types of cells in an organism. In other words, somatic cells can be cells that have differentiated sufficiently that they may not naturally generate cells of all three germ layers of the body, e.g. ectoderm, mesoderm and endoderm. For example, somatic cells can include both neurons and neural progenitors, the latter of which is able to naturally give rise to all or some cell types of the central nervous system but cannot give rise to cells of the mesoderm or endoderm lineages.

In certain examples, the stem cells can be undifferentiated (e.g. a cell not committed to a specific lineage) prior to exposure to at least one differentiation factor or composition according to the methods as disclosed herein, whereas in other examples it can be desirable to differentiate the stem cells to one or more intermediate cell types prior to exposure of the at least one differentiation factor or composition described herein. For example, the stems cells can display morphological, biological or physical characteristics of undifferentiated cells that can be used to distinguish them from differentiated cells of embryo or adult origin. In some examples, undifferentiated cells can appear in the two dimensions of a microscopic view in colonies of cells with high nuclear/cytoplasmic ratios and prominent nucleoli. The stem cells can be themselves (for example, without substantially any undifferentiated cells being present) or can be used in the presence of differentiated cells. In certain examples, the stem cells can be cultured in the presence of) suitable nutrients and optionally other cells such that the stem cells can grow and optionally differentiate. For example, embryonic fibroblasts or fibroblast-like cells can be present in the culture to assist in the growth of the stem cells. The fibroblast can be present during one stage of stem cell growth but not necessarily at all stages. For example, the fibroblast can be added to stem cell cultures in a first culturing stage and not added to the stem cell cultures in one or more subsequent culturing stages.

Stem cells used in all aspects of the present disclosure can be any cells derived from any kind of tissue (for example embryonic tissue such as fetal or pre-fetal tissue, or adult tissue), which stem cells can have the characteristic of being capable under appropriate conditions of producing progeny of different cell types, e.g. derivatives of all of at least one of the 3 germinal layers (endoderm, mesoderm, and ectoderm). These cell types can be provided in the form of an established cell line, or they can be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, FISF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo. In some embodiments, the source of human stem cells or pluripotent stem cells used for chemically-induced differentiation into mature, insulin positive cells do not involve destroying a human embryo.

In another example, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells that can be used in the methods provided herein can also include embryonic cells of various types, exemplified by human embryonic stem (hES) cells, as described by Thomson et al, (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also applicable to the methods provided herein can be lineage committed stem cells, such as mesodermal stem cells and other early cardiogenic cells (see Reyes et al, (2001) Blood 98:2615-2625; Eisenberg & Bader (1996) Circ Res. 78(2):205-16; etc.) The stem cells can be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein. In some embodiments, a human embryo is not destroyed for the source of pluripotent cell used on the methods and compositions as disclosed herein.

A mixture of cells from a suitable source of endothelial, muscle, and/or neural stem cells can be harvested from a mammalian donor for the purpose of the present disclosure. A suitable source is the hematopoietic microenvironment. For example, circulating peripheral blood, preferably mobilized (e.g., recruited), may be removed from a subject. In an embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the pancreatic α, β and/or δ cell as described herein can be derived from one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

### Reprogramming

The term “reprogramming” as used herein can refer to the process that alters or reverses the differentiation state of a somatic cell. The cell can either be partially or terminally differentiated prior to the reprogramming. Reprogramming can encompass complete reversion of the differentiation state of a somatic cell to a pluripotent cell. Such complete reversal of differentiation can produce an induced pluripotent (iPS) cell. Reprogramming as used herein can also encompass partial reversion of a cells differentiation state, for example to a multipotent state or to a somatic cell that is neither pluripotent or multipotent, but is a cell that has lost one or more specific characteristics of the differentiated cell from which it arises, e.g. direct reprogramming of a differentiated cell to a different somatic cell type. Reprogramming can involve alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the term “reprogramming factor” can refer to a molecule that is associated with cell “reprogramming,” that is, differentiation, and/or de-differentiation, and/or transdifferentiation, such that a cell converts to a different cell type or phenotype. Reprogramming factors generally affect expression of genes associated with cell differentiation, de-differentiation and/or transdifferentiation. Transcription factors are examples of reprogramming factors.

The term “differentiation” and their grammatical equivalents as used herein can refer to the process by which a less specialized cell (e.g., a more naive cell with a higher cell potency) becomes a more specialized cell type (e.g., a less naive cell with a lower cell potency); and that the term “de-differentiation” can refer to the process by which a more specialized cell becomes a less specialized cell type (e.g., a more naive cell with a higher cell potency); and that the term “transdifferentiation” refers to the process by which a cell of a particular cell type converts to another cell type without significantly changing its “cell potency” or “naivety” level. Without wishing to be bound by theory, it is thought that cells “transdifferentiate” when they convert from one lineage-committed cell type or terminally differentiated cell type to another lineage-committed cell type or terminally differentiated cell type, without significantly changing their “cell potency” or “naivety” level.

As used herein, the term “cell potency” is to be understood as referring to the ability of a cell to differentiate into cells of different lineages. For example, a pluripotent cell (e.g., a stem cell) has the potential to differentiate into cells of any of the three germ layers, that is, endoderm (interior stomach lining, gastrointestinal tract, the lungs), mesoderm (muscle, bone, blood, urogenital), or ectoderm (epidermal tissues and nervous system), and accordingly has high cell potency; a multipotent cell (e.g., a stem cell or an induced stem cell of a certain type) has the ability to give rise to cells from a multiple, but limited, number of lineages (such as hematopoietic stem cells, cardiac stem cells, or neural stem cells, etc.) comparatively has a lower cell potency than pluripotent cells. Cells that are committed to a particular lineage or are terminally differentiated can have yet a lower cell potency. Specific examples of transdifferentiation known in the art include the conversion of e.g., from pancreatic exocrine cells to beta cells etc.

Accordingly, the cell may be caused to differentiate into a more naive cell (e.g., a terminally differentiated cell may be differentiated to be multipotent or pluripotent); or the cell may be caused to de-differentiate into a less naive cell (e.g., a multipotent or pluripotent cell can be differentiated into a lineage-committed cell or a terminally differentiated cell). However, in an embodiment, the cell may be caused to convert or transdifferentiate from one cell type (or phenotype) to another cell type (or phenotype), for example, with a similar cell potency level. Accordingly, in an embodiment of the present disclosure, the inducing steps of the present disclosure can reprogram the cells of the present disclosure to differentiate, de-differentiate and/or transdifferentiate. In an embodiment of the present disclosure, the inducing steps of the present disclosure may reprogram the cells to transdifferentiate.

Methods of reprogramming or inducing a particular type of cell to become another type of cell, for example, by differentiation, de-differentiation and/or transdifferentiation using one or more exogenous polynucleotide or polypeptide reprogramming factors are known to the person skilled in the art. Such methods may rely on the introduction of genetic material encoding one or more transcription factor(s) or other polypeptide(s) associated with cell reprogramming. For example, PDX1, Ngn3 and MafA, or functional fragments thereof are all known to encode peptides that can induce cell differentiation, de-differentiation and/or transdifferentiation of the cells of the present disclosure. In some methods known to the person skilled in the art, exogenous polypeptides (e.g. recombinant polypeptides) encoded by reprogramming genes (such as the above genes) are contacted with the cells to induce, for example, cells of the present disclosure. The person skilled in the art will appreciate that other genes may be associated with reprogramming of cells, and exogenous molecules encoding such genes (or functional fragments thereof) and the encoded polypeptides are also considered to be polynucleotide or polypeptide reprogramming factors (e.g. polynucleotides or polypeptides that in turn affect expression levels of another gene associated with cell reprogramming). For example, it has been shown that the introduction of exogenous polynucleotide or polypeptide epigenetic gene silencers that decrease p53 inactivation increase the efficiency of inducing induced pluripotent stem cells (iPSC). Accordingly, exogenous polynucleotides or polypeptides encoding epigenetic silencers and other genes or proteins that may be directly or indirectly involved in cell reprogramming or increasing cell programming efficiency would be considered to constitute an exogenous polynucleotide or polypeptide reprogramming factor. The person skilled in the art will appreciate that other methods of influencing cell reprogramming exist, such as introducing RNAi molecules (or genetic material encoding RNAi molecules) that can knock down expression of genes involved in inhibiting cell reprogramming. Accordingly, any exogenous polynucleotide molecule or polypeptide molecule that is associated with cell reprogramming, or enhances cell reprogramming, is to be understood to be an exogenous polynucleotide or polypeptide reprogramming factor as described herein.

Accordingly, in an embodiment, the present disclosure does not involve a culturing step of the cell(s) with one or more exogenous polynucleotide or polypeptide reprogramming factor(s). Accordingly, in an embodiment, the method of the present disclosure does not involve the introduction of one or more exogenous polynucleotide or polypeptide reprogramming factor(s), e.g., by introducing transposons, viral transgenic vectors (such as retroviral vectors), plasmids, mRNA, miRNA, peptides, or fragments of any of these molecules, that are involved in producing induced α, β and/or δ cells or, otherwise, inducing cells of the present disclosure to differentiate, de-differentiation and/or transdifferentiate.

That is, in an embodiment, the method occurs in the absence of one or more exogenous polynucleotide or polypeptide reprogramming factor(s) (e.g., activin A). Accordingly, it is to be understood that in an embodiment, the method of the present disclosure utilizes small molecules to reprogram cells, without the addition of polypeptide transcription factors; other polypeptide factors specifically associated with inducing differentiation, de-differentiation, and/or transdifferentiation; polynucleotide sequences encoding polypeptide transcription factors, polynucleotide sequences encoding other polypeptide factors specifically associated with inducing differentiation, de-differentiation, and/or transdifferentiation; mRNA; interference RNA; microRNA and fragments thereof.

### Methods of Generating Stem Cell Derived Islet Cells

Provided herein are methods of generating SC-islet cells (e.g., non-native pancreatic β cells). Examples of detailed protocols of generating endocrine cells from stem cells to provide at least one SC-islet cell (e.g., SC-beta cell) are described in U.S. Pate Application Publication Nos. US20150240212, US20150218522, US20210198632, and US 20220090020, PCT Publications WO2022/147056 and WO2022192300, and US Pat. No. 11,466,256, each of which is herein incorporated by reference in its entirety.

The endoderm can give rise to digestive and respiratory tracts, thyroid, liver, and pancreas. A representative disease of endoderm lineages is type 1 diabetes resulting from destruction of the insulin-producing β cells. Generation of functional β cells from human pluripotent stem cells (hPSC) in vitro can be a practical, renewable cell source for replacement cell therapy for type 1 diabetes. The embryotic stem (ES) cells that are generated from the inner cell mass of blastocyst-stage embryos represent a promising source of cells for transplantation or cell-based therapy of any damaged cells. They can be maintained in culture, renew for themselves, and proliferate unlimitedly as undifferentiated ES cells. The ES cells are capable of differentiating into all cell types of the body as the ectoderm, mesoderm, and endoderm lineage cells or tissues. The major benefit of ES cells is stable self-renewal in culture and the potential to differentiate.

The definitive endoderm can be generated in vivo from the inner cell mass by the process of gastrulation of embryogenesis, in which epiblast cells are instructed to form the three germ layers. Definitive endoderm can give rise to diverse cells and tissues that contribute to vital organs as the pancreatic β cells, liver hepatocytes, lung alveolar cells, thyroid, thymus, and the epithelial lining of the alimentary and respiratory tract. It is different from the primitive endoderm of extraembryonic tissues, which can give rise to the visceral and parietal endoderm. The definitive endoderm derived from ES cells is theoretically capable of becoming any endoderm derivatives and directing ES cells into the endoderm lineage is a prerequisite for generating therapeutic endoderm derivatives.

Precise patterning of anterior-posterior axis of the definitive endoderm can eventually form the primitive gut tube. The definitive endoderm-derived primitive gut tube induces the pharynx, esophagus, stomach, duodenum, small and large intestine along the anterior-posterior axis as well as associated organs, including pancreas, lung, thyroid, thymus, parathyroid, and liver. The anterior portion of the foregut of the primitive gut tube becomes lung, thyroid, esophagus, and stomach. The pancreas, liver, and duodenum originate from the posterior portion of the foregut. The midgut and hindgut of primitive gut tube gives rise to the small and large intestine. The anterior foregut expresses developmental markers, NK2 homeobox 1 (NKX2-1) and SRY (sex determining region Y)-box 2 (SOX2); the posterior foregut expresses hematopoietically expressed homeobox (HHEX), pancreatic and duodenal homeobox 1 (PDX1), one cut homeobox 1 (ONECUT1, known as HNF6), and hepatocyte nuclear factor 4 alpha (HNF4A); and the midgut/hindgut expresses caudal type homeobox 1 (CDX1), caudal type homeobox 2 (CDX2), and motor neuron and pancreas homeobox 1 (MNX1) (3, 19, 20).

The successful differentiation to pancreatic α, β and/or δ cells can involve differentiated cells synthesizing and secreting physiologically appropriate amounts of insulin. An exemplary stepwise protocol directing hPSC cell differentiation is developed, which entails differentiation processes that recapitulates the major stages of normal pancreatic endocrine development. The differentiation of hPSC cells to hormone-expressing pancreatic endocrine cells is conducted by transitioning hPSC cells through major stages of embryonic development; differentiation to mesendoderm and definitive endoderm, establishment of the primitive gut endoderm, patterning of the posterior foregut, and specification and maturation of pancreatic endoderm and endocrine precursors. Through these stages, hPSC cells can obtain pancreatic endocrine phenotype and ability of glucose responsive insulin secretion in vitro.

Generally, the at least one pancreatic α, β and/or δ cell or precursor thereof, e.g., pancreatic progenitors produced according to the methods disclosed herein can comprise a mixture or combination of different cells, e.g., for example a mixture of cells such as a PDX1-positive pancreatic progenitors, pancreatic progenitors co-expressing PDX1 and NKX6-1, a Ngn3-positive endocrine progenitor cell, an insulin-positive endocrine cell (e.g., NKX6.1-positive, ISL1-positive cells, or β-like cells), and/or other pluripotent or stem cells.

The at least one pancreatic α, β and/or δ cell or precursor thereof can be produced according to any suitable culturing protocol to differentiate a stem cell or pluripotent cell to a desired stage of differentiation. In some embodiments, the at least one pancreatic α, β and/or δ cell or the precursor thereof are produced by culturing at least one pluripotent cell for a period of time and under conditions suitable for the at least one pluripotent cell to differentiate into the at least one pancreatic α, β and/or δ cell or the precursor thereof.

In some embodiments, the at least one pancreatic α, β and/or δ cell or precursor thereof is a substantially pure population of pancreatic α, β and/or δ cells or precursors thereof. In some embodiments, a population of pancreatic α, β and/or δ cells or precursors thereof comprises a mixture of pluripotent cells or differentiated cells. In some embodiments, a population pancreatic α, β and/or δ cells or precursors thereof are substantially free or devoid of embryonic stem cells or pluripotent cells or iPS cells.

In some embodiments, a somatic cell, e.g., a fibroblast, can be isolated from a subject, for example as a tissue biopsy, such as, for example, a skin biopsy, and reprogrammed into an induced pluripotent stem cell for further differentiation to produce the at least one SC-β cell or precursor thereof for use in the compositions and methods described herein. In some embodiments, a somatic cell, e.g., a fibroblast, is maintained in culture by methods known by one of ordinary skill in the art, and in some embodiments, propagated prior to being converted into pancreatic α, β and/or δ cells by the methods as disclosed herein.

In some embodiments, the at least one pancreatic α, β and/or δ cell or precursor thereof are maintained in culture by methods known by one of ordinary skill in the art, and in some embodiments, propagated prior to being converted into pancreatic α, β and/or δ cells by the methods as disclosed herein.

Further, at least one pancreatic α, β and/or δ cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian at least one pancreatic α, β and/or δ cell or precursor thereof but it should be understood that all of the methods described herein can be readily applied to other cell types of at least one pancreatic α, β and/or δ cell or precursor thereof. In some embodiments, the at least one SC-β cell or precursor thereof is derived from a human individual.

### Stages Of Differentiation

In some embodiments, pancreatic differentiation as disclosed herein is carried out in a step-wise manner. In the step-wise progression, “Stage 1” or “S1” refers to the first step in the differentiation process, the differentiation of pluripotent stem cells into cells expressing markers characteristic of definitive endoderm cells (“DE”, “Stage 1 cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor 1 cells (“PP1”, “Stage 3 cells” or “S3 cells”). “Stage 4” refers to the fourth step, the differentiation of cells expressing markers characteristic of pancreatic progenitor 1 cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells (e.g., PDX.1⁺, NKX6.1⁺) into cells expressing markers characteristic of pancreatic endoderm cells and/or pancreatic endocrine progenitor cells (e.g., insulin⁺) (“EN”, “Stage 5 cells” or “S5 cells”). “Stage 6” refers to the differentiation of cells expressing markers characteristic of pancreatic endocrine progenitor cells (e.g., insulin) into cells expressing markers characteristic of pancreatic endocrine β cells (“SC-β cells”) or pancreatic endocrine α cells (“SC-a cells”). It should be appreciated, however, that not all cells in a particular population progress through these stages at the same rate, i.e., some cells may have progressed less, or more, down the differentiation pathway than the majority of cells present in the population. For example, in some embodiments, SC-β cells can be identified during stage 5, at the conclusion of stage 5, at the beginning of stage 6, etc. Examples of methods of making cells of any one of stages 1-6 are provided in, for example, US Pat. No. 10,030,229; US Pat. No. 10,443,042; US Patent No. 11,466,256, published applications US 20200332262 US20150240212, US20150218522, US20210198632, and US 20220090020; and PCT Publications WO2022/147056 and WO2022192300, each of which is incorporated by reference in its entirety.

### Definitive Endoderm Cells

Aspects of the disclosure involve definitive endoderm cells. Definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), PDX1-positive, NKX6.1-negative pancreatic progenitor cells (stage 3), NKX6.1-positive pancreatic progenitor cells (stage 4), or Ngn3-positive endocrine progenitor cells or insulin-positive endocrine cells (stage 5), followed by induction or maturation to SC-β cells (stage 6).

In some cases, definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

Any growth factor from the TGF-β superfamily capable of inducing the pluripotent stem cells to differentiate into definitive endoderm cells (e.g., alone, or in combination with a WNT signaling pathway activator) can be used in the method provided herein. In some cases, the growth factor from the TGF-β superfamily comprises Activin A. In some cases, the growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily) can be used in the method provided herein. In some cases, the WNT signaling pathway activator comprises CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, or TWS 119. In some embodiments, the WNT signaling pathway activator comprises CHIR99021. In some cases, the WNT signaling pathway activator comprises Wnt3a recombinant protein.

In some cases, differentiating at least some pluripotent cells in a population into definitive endoderm cells is achieved by a process of contacting a population of pluripotent cells with i) Activin A, and ii) CHIR99021 for a suitable period of time, e.g., about 2 days, about 3 days, about 4 days, or about 5 days to induce the differentiation of at least some of the pluripotent cells in the population into definitive endoderm cells, wherein the definitive endoderm cells express at least one marker characteristic of definitive endoderm. In some embodiments, the population of pluripotent cells is contacted with the growth factor from the TGF-β superfamily (e.g., activin A) and the WNT signaling pathway activator (e.g., CHIR99021) for 1, 2, or 3 days (e.g., 1 day), and the population of pluripotent cells is further contacted with the TGF-β superfamily (e.g., activin A) for 1, 2, 3, or 4 days (e.g., 2 days) in the absence of the WNT signaling pathway activator (e.g., CHIR99021).

In some examples, the method comprises differentiating pluripotent cells into definitive endoderm cells by contacting a population of pluripotent cells with a suitable concentration of the growth factor from the TGF-β superfamily (e.g., Activin A), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some cases, the method comprises use of about 100 ng/mL Activin A for differentiation of pluripotent cells into definitive endoderm cells. In some cases, the method comprises use of about 200 ng/mL Activin A for differentiation of pluripotent cells into definitive endoderm cells.

In some examples, the method comprises differentiating pluripotent cells into definitive endoderm cells by contacting a population of pluripotent cells with a suitable concentration of the WNT signaling pathway activator (e.g., CHIR99021), such as, about 0.01 µM, about 0.05 µM, about 0.1 µM, about 0.2 µM, about 0.5 µM, about 0.8 µM, about 1 µM, about 1.5 µM, about 2 µM, about 2.5 µM, about 3 µM, about 3.5 µM, about 4 µM, about 5 µM, about 8 µM, about 10 µM, about 12 µM, about 15 µM, about 20 µM, about 30 µM, about 50 µM, about 100 µM, or about 200 µM. In some cases, the method comprises use of about 2 µM CHIR99021 for differentiation of pluripotent cells into definitive endoderm cells. In some cases, the method comprises use of about 5 µM CHIR99021 for differentiation of pluripotent cells into definitive endoderm cells.

In some cases, a definitive endoderm cell produced by the methods as disclosed herein expresses at least one marker selected from the group consisting of: Nodal, Tmprss2, Tmem30b, St14, Spink3, Sh3gl2, Ripk4, Rab1S, Npnt, Clic6, Cldn5, Cacna1b, Bnip1, Anxa4, Emb, FoxA1, Sox17, and Rbm35a, wherein the expression of at least one marker is upregulated by a statistically significant amount in the definitive endoderm cell relative to the pluripotent stem cell from which it was derived. In some cases, a definitive endoderm cell produced by the methods as disclosed herein does not express by a statistically significant amount at least one marker selected the group consisting of: Gata4, SPARC, AFP and Dab2 relative to the pluripotent stem cell from which it was derived. In some cases, a definitive endoderm cell produced by the methods as disclosed herein does not express a statistically significant amount at least one marker selected the group consisting of: Zic1, Pax6, Flk1 and CD31 relative to the pluripotent stem cell from which it was derived. In some cases, a definitive endoderm cell produced by the methods as disclosed herein has a higher level of phosphorylation of Smad2 by a statistically significant amount relative to the pluripotent stem cell from which it was derived. In some cases, a definitive endoderm cell produced by the methods as disclosed herein has the capacity to form gut tube in vivo. In some cases, a definitive endoderm cell produced by the methods as disclosed herein can differentiate into a cell with morphology characteristic of a gut cell, and wherein a cell with morphology characteristic of a gut cell expresses FoxA2 and/or Claudin6. In some cases, a definitive endoderm cell produced by the methods as disclosed herein can be further differentiated into a cell of endoderm origin.

In some cases, a population of pluripotent stem cells are cultured in the presence of at least one β cell differentiation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some cases, a β cell differentiation factor as described herein can be present in the culture medium of a population of pluripotent stem cells or may be added in bolus or periodically during growth (e.g. replication or propagation) of the population of pluripotent stem cells. In certain examples, a population of pluripotent stem cells can be exposed to at least one β cell differentiation factor prior to any differentiation. In other examples, a population of pluripotent stem cells may be exposed to at least one β cell differentiation factor during the first stage of differentiation.

### Primitive Gut Tube Cells

Aspects of the disclosure involve primitive gut tube cells. Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to PDX1-positive, NKX6.1-negative pancreatic progenitor cells, NKX6.1-positive pancreatic progenitor cells, Ngn3-positive endocrine progenitor cells, insulin-positive endocrine cells, followed by induction or maturation to SC-β cells.

In some cases, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

Any growth factor from the FGF family capable of inducing definitive endoderm cells to differentiate into primitive gut tube cells (e.g., alone, or in combination with other factors) can be used in the method provided herein. In some cases, the at least one growth factor from the FGF family comprises keratinocyte growth factor (KGF). In some cases, the at least one growth factor from the FGF family comprises FGF2. In some cases, the at least one growth factor from the FGF family comprises FGF8B. In some cases, the at least one growth factor from the FGF family comprises FGF 10. In some cases, the at least one growth factor from the FGF family comprises FGF21.

In some cases, primitive gut tube cells can be obtained by differentiating at least some definitive endoderm cells in a population into primitive gut tube cells, e.g., by contacting definitive endoderm cells with KGF for a certain period of time, e.g., about 1 day, about 2 days, about 3 days, about 4 days, or about 5 days, to induce the differentiation of at least some of the definitive endoderm cells into primitive gut tube cells.

In some cases, the method comprises differentiating definitive endoderm cells into primitive gut tube cells by contacting definitive endoderm cells with a suitable concentration of the growth factor from the FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some cases, the method comprises use of about 50 ng/mL KGF for differentiation of definitive endoderm cells into primitive gut tube cells. In some cases, the method comprises use of about 100 ng/mL KGF for differentiation of definitive endoderm cells into primitive gut tube cells.

### PDX1-Positive Pancreatic Progenitor Cells

Aspects of the disclosure involve PDX1-positive, NKX6.1-negative pancreatic progenitor cells. PDX1-positive, NKX6.1-negative pancreatic progenitor cells of use herein can be derived from any source or generated in accordance with any suitable protocol. In some aspects, primitive gut tube cells are differentiated to PDX1-positive, NKX6.1-negative pancreatic progenitor cells. In some aspects, the PDX1-positive, NKX6.1-negative pancreatic progenitor cells are further differentiated, e.g., NKX6.1-positive pancreatic progenitor cells, Ngn3-positive endocrine progenitor cells, insulin-positive endocrine cells, followed by induction or maturation to pancreatic α, β and/or δ cells,

In some aspects, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; vi) at least one protein kinase C activator, and vii) ROCK inhibitor to induce the differentiation of at least some of the primitive gut tube cells into PDX1-positive, NKX6.1-negative pancreatic progenitor cells, wherein the PDX1-positive, NKX6.1-negative pancreatic progenitor cells express PDX1.

In some aspects, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; and vi) at least one protein kinase C activator, to induce the differentiation of at least some of the primitive gut tube cells into PDX1-positive, NKX6.1-negative pancreatic progenitor cells, wherein the PDX1-positive, NKX6.1-negative pancreatic progenitor cells express PDX1.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor, ii) at least one growth factor from the FGF family, iii) at least one SHH pathway inhibitor, iv) at least one retinoic acid (RA) signaling pathway activator; and v) at least one protein kinase C activator, to induce the differentiation of at least some of the primitive gut tube cells into PDX1-positive, NKX6.1-negative pancreatic progenitor cells, wherein the PDX1-positive, NKX6.1-negative pancreatic progenitor cells express PDX1.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one SHH pathway inhibitor, ii) at least one retinoic acid (RA) signaling pathway activator; and iii) at least one protein kinase C activator, wherein the PDX1-positive, NKX6.1-negative pancreatic progenitor cells express PDX1.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into PDX1-positive, NKX6.1-negative pancreatic progenitor cells, wherein the PDX1-positive, NKX6.1-negative pancreatic progenitor cells express PDX1.

Any BMP signaling pathway inhibitor capable of inducing primitive gut tube cells to differentiate into PDX1-positive, NKX6.1-negative pancreatic progenitor cells (e.g., alone, or with any combination of a growth factor from TGF-β superfamily, at least one growth factor from the FGF family, at least one SHH pathway inhibitor, at least one retinoic acid signaling pathway activator, at least one protein kinase C activator, and ROCK inhibitor) can be used in the method provided herein. In some cases, the BMP signaling pathway inhibitor comprises LDN193189 or DMH-1. In some examples, the method comprises contacting primitive gut tube cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 µM. In some examples, the method comprises contacting primitive gut tube cells with a concentration of BMP signaling pathway inhibitor (e.g., DMH-1), such as, about 0.01 µM, about 0.02 µM, about 0.05 µM, about 0.1 µM, about 0.2 µM, about 0.5 µM, about 0.8 µM, about 1 µM, about 1.2 µM, about 1.5 µM, about 1.75 µM, about 2 µM, about 2.2 µM, about 2.5 µM, about 2.75 µM, about 3 µM, about 3.25 µM, about 3.5 µM, about 3.75 µM, about 4 µM, about 4.5 µM, about 5 µM, about 8 µM, about 10 µM, about 15 µM, about 20 µM, about 30 µM, about 40 µM, about 50 µM, or about 100 µM.

Any growth factor from the TGF-β superfamily capable of inducing primitive gut tube cells to differentiate into PDX1-positive, NKX6.1-negative pancreatic progenitor cells (e.g., alone, or with any combination of at least one BMP signaling pathway inhibitor, a growth factor from the FGF family, at least one SHH pathway inhibitor, at least one retinoic acid signaling pathway activator, at least one protein kinase C activator, and ROCK inhibitor) can be used. In some cases, the growth factor from TGF-β family comprises Activin A. In some cases, the growth factor from TGF-β family comprises Activin A or GDF8. In some examples, the method comprises contacting primitive gut tube cells with a concentration of a growth factor from TGF-β superfamily (e.g., Activin A), such as, about 5 ng/mL, about 7.5 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 35 ng/mL, about 40 ng/mL, about 50 ng/mL, or about 100 ng/mL.

Any growth factor from the FGF family capable of inducing primitive gut tube cells to differentiate into PDX1-positive, NKX6.1-negative pancreatic progenitor cells (e.g., alone, or with any combination of at least one BMP signaling pathway inhibitor, a growth factor from TGF-β superfamily, at least one SHH pathway inhibitor, at least one retinoic acid signaling pathway activator, at least one protein kinase C activator, and ROCK inhibitor) can be used. In some cases, the at least one growth factor from the FGF family comprises keratinocyte growth factor (KGF). In some cases, the at least one growth factor from the FGF family is selected from the group consisting of FGF2, FGF8B, FGF 10, and FGF21. In some examples, the method comprises contacting primitive gut tube cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting primitive gut tube cells with a concentration of a growth factor from FGF family (e.g., KGF), such as, 10-200 ng/mL, 10-150 ng/mL, 10-100 ng/mL, 25-75 ng/mL, 40-60 ng/mL, or 45-55 ng/mL.

Any SHH pathway inhibitor capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, a growth factor from TGF-β superfamily, at least one retinoic acid signaling pathway activator, at least one protein kinase C activator, and ROCK inhibitor) can be used. In some cases, the SHH pathway inhibitor comprises Sant1. In some examples, the method comprises contacting primitive gut tube cells with a concentration of a SHH pathway inhibitor (e.g., Sant1), such as, about 0.001 µM, about 0.002 µM, about 0.005 µM, about 0.01 µM, about 0.02 µM, about 0.03 µM, about 0.05 µM, about 0.08 µM, about 0.1 µM, about 0.12 µM, about 0.13 µM, about 0.14 µM, about 0.15 µM, about 0.16 µM, about 0.17 µM, about 0.18 µM, about 0.19 µM, about 0.2 µM, about 0.21 µM, about 0.22 µM, about 0.23 µM, about 0.24 µM, about 0.25 µM, about 0.26 µM, about 0.27 µM, about 0.28 µM, about 0.29 µM, about 0.3 µM, about 0.31 µM, about 0.32 µM, about 0.33 µM, about 0.34 µM, about 0.35 µM, about 0.4 µM, about 0.45 µM, about 0.5 µM, about 0.6 µM, about 0.8 µM, about 1 µM, about 2 µM, or about 5 µM. In some examples, the method comprises contacting primitive gut tube cells with a concentration of a SHH pathway inhibitor (e.g., Sant1), 50-1000 nM, 50-500 nM, 50-300 nM, 100-300 nM, 200-300 nM, 200-500 nM, or 225-275 nM.

Any RA signaling pathway activator capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, at least one protein kinase C activator, and ROCK inhibitor) can be used. In some cases, the RA signaling pathway activator comprises retinoic acid. In some examples, the method comprises contacting primitive gut tube cells with a concentration of an RA signaling pathway activator (e.g., retinoic acid), such as, about 0.02 µM, about 0.1 µM, about 0.2 µM, about 0.25 µM, about 0.3 µM, about 0.4 µM, about 0.45 µM, about 0.5 µM, about 0.55 µM, about 0.6 µM, about 0.65 µM, about 0.7 µM, about 0.75 µM, about 0.8 µM, about 0.85 µM, about 0.9 µM, about 1 µM, about 1.1 µM, about 1.2 µM, about 1.3 µM, about 1.4 µM, about 1.5 µM, about 1.6 µM, about 1.7 µM, about 1.8 µM, about 1.9 µM, about 2 µM, about 2.1 µM, about 2.2 µM, about 2.3 µM, about 2.4 µM, about 2.5 µM, about 2.6 µM, about 2.7 µM, about 2.8 µM, about 3 µM, about 3.2 µM, about 3.4 µM, about 3.6 µM, about 3.8 µM, about 4 µM, about 4.2 µM, about 4.4 µM, about 4.6 µM, about 4.8 µM, about 5 µM, about 5.5 µM, about 6 µM, about 6.5 µM, about 7 µM, about 7.5 µM, about 8 µM, about 8.5 µM, about 9 µM, about 9.5 µM, about 10 µM, about 12 µM, about 14 µM, about 15 µM, about 16 µM, about 18 µM, about 20 µM, about 50 µM, or about 100 µM. In some examples, the method comprises contacting primitive gut tube cells with a concentration of an RA signaling pathway activator (e.g., retinoic acid), such as, 0.2-5 µM, 0.8-3 µM, 0.8-2.5 µM, 1-2.5 µM, 1.5-2.5 µM, 1.8-2.2 µM, or 1.9-2.1 µM.

Any PKC activator capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, at least one RA signaling pathway activator, and ROCK inhibitor) can be used. In some cases, the PKC activator comprises PdBU. In some cases, the PKC activator comprises TPPB. In some examples, the method comprises contacting primitive gut tube cells with a concentration of a PKC activator (e.g., PdBU or TPPB), such as, about 10 nM, 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 µM, 10 µM, about 20 µM, about 50 µM, about 75 µM, about 80 µM, about 100 µM, about 120 µM, about 140 µM, about 150 µM, about 175 µM, about 180 µM, about 200 µM, about 210 µM, about 220 µM, about 240 µM, about 250 µM, about 260 µM, about 280 µM, about 300 µM, about 320 µM, about 340 µM, about 360 µM, about 380 µM, about 400 µM, about 420 µM, about 440 µM, about 460 µM, about 480 µM, about 500 µM, about 520 µM, about 540 µM, about 560 µM, about 580 µM, about 600 µM, about 620 µM, about 640 µM, about 660 µM, about 680 µM, about 700 µM, about 750 µM, about 800 µM, about 850 µM, about 900 µM, about 1 mM, about 2 mM, about 3 mM, about 4 mM, or about 5 mM. In some embodiments, the method comprises contacting primitive gut tube cells with a concentration of a PKC activator (e.g., PdBU or TPPB) of 10 nM-1 mM, 10 nM-500 µM, 10 nM-1 µM, 10-800 nM, 100-900 nM, 300-800 nM, 300-600 nM, 400-600 nM, 450-550 nM, or about 500 nM. In some embodiments, primitive gut tube cells are not treated with a PKC activator (e.g., PDBU).

Any ROCK inhibitor capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, PKC activator, and at least one RA signaling pathway activator) can be used. In some cases, the ROCK inhibitor comprises Thiazovivin, Y-27632, Fasudil/HA1077, or H-1152. In some cases, the ROCK inhibitor comprises Y-27632. In some cases, the ROCK inhibitor comprises Thiazovivin. In some examples, the method comprises contacting primitive gut tube cells with a concentration of a ROCK inhibitor (e.g., Y-27632 or Thiazovivin), such as, about 0.2 µM, about 0.5 µM, about 0.75 µM, about 1 µM, about 2 µM, about 3 µM, about 4 µM, about 5 µM, about 6 µM, about 7 µM, about 7.5 µM, about 8 µM, about 9 µM, about 10 µM, about 11 µM, about 12 µM, about 13 µM, about 14 µM, about 15 µM, about 16 µM, about 17 µM, about 18 µM, about 19 µM, about 20 µM, about 21 µM, about 22 µM, about 23 µM, about 24 µM, about 25 µM, about 26 µM, about 27 µM, about 28 µM, about 29 µM, about 30 µM, about 35 µM, about 40 µM, about 50 µM, or about 100 µM. In some examples, the method comprises contacting primitive gut tube cells with a concentration of a ROCK inhibitor (e.g., Y-27632 or Thiazovivin), such as, 0.2-5 µM, 0.8-3 µM, 1-4 µM, 1.5-4 µM, 1.8-3.5 µM, 2-3 µM, 2.4-2.6 µM.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with retinoic acid, KGF, Sant1, DMH-1, PdBU, thiazovivin, and Activin A, for a suitable period of time, e.g., about 1 day, about 2 days, about 3 days, or about 4 days. In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with retinoic acid, KGF, Sant1, DMH-1, PdBU, thiazovivin, and Activin A, for about 2 days.

In some embodiments, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor (e.g., DMH-1), ii) a growth factor from TGF-β superfamily (e.g., activin A), iii) at least one growth factor from the FGF family (e.g., KGF), iv) at least one SHH pathway inhibitor (e.g., Sant1), v) at least one retinoic acid (RA) signaling pathway activator (e.g., retinoic acid); vi) at least one protein kinase C activator (e.g., PDBU), and vii) at least one ROCK inhibitor (e.g., thiazovivin) for 1, 2, 3, 4, or 4 days (e.g., 1 day), and then i) a growth factor from TGF-β superfamily (e.g., activin A), ii) at least one growth factor from the FGF family (e.g., KGF), iii) at least one SHH pathway inhibitor (e.g., Sant1), iv) at least one retinoic acid (RA) signaling pathway activator (e.g., retinoic acid); v) at least one protein kinase C activator (e.g., PDBU), and vi) at least one ROCK inhibitor (e.g., thiazovivin) for 1, 2, 3, 4, or 5 day (e.g., 1 day) in the absence of a BMP signaling pathway inhibitor (e.g., DMH-1).

### NKX6.1-Positive Pancreatic Progenitor Cells

Aspects of the disclosure involve PDX1-positive, NKX6.1-positive pancreatic progenitor cells. PDX1-positive, NKX6.1-positive pancreatic progenitor cells of use herein can be derived from any source or generated in accordance with any suitable protocol. In some aspects, PDX1-positive, NKX6.1-negative pancreatic progenitor cells are differentiated to PDX-1 positive, NKX6.1-positive pancreatic progenitor cells. In some aspects, the PDX-1 positive, NKX6.1-positive pancreatic progenitor cells are further differentiated, e.g., to Ngn3-positive endocrine progenitor cells, or insulin-positive endocrine cells, followed by induction or maturation to SC-β cells.

In some aspects, a method of producing a PDX-1 positive, NKX6.1-positive pancreatic progenitor cell from a PDX1-positive, NKX6.1-negative pancreatic progenitor cell comprises contacting a population of cells (e.g., under conditions that promote cell clustering and/or promoting cell survival) comprising PDX1-positive, NKX6.1-negative pancreatic progenitor cells with at least two β cell-differentiation factors comprising a) at least one growth factor from the fibroblast growth factor (FGF) family, b) a sonic hedgehog pathway inhibitor, and optionally c) a retinoic acid (RA) signaling pathway activator, to induce the differentiation of at least one PDX1-positive, NKX6.1-negative pancreatic progenitor cell in the population into PDX1-positive, NKX6.1-positive pancreatic progenitor cells, wherein the PDX1-positive, NKX6.1-positive pancreatic progenitor cells expresses NKX6.1.

In some cases, the PDX1-positive, NKX6.1-positive pancreatic progenitor cells are obtained by contacting PDX1-positive, NKX6.1-negative pancreatic progenitor cells with i) at least one growth factor from the FGF family, ii) at least one SHH pathway inhibitor, and optionally iii) a RA signaling pathway activator, to induce the differentiation of at least some of the PDX1-positive, NKX6.1-negative pancreatic progenitor cells into PDX1-positive, NKX6.1-positive pancreatic progenitor cells, wherein the PDX1-positive, NKX6.1- positive pancreatic progenitor cells expresses PDX1 and NKX6.1.

In some cases, the PDX1-positive, NKX6.1-positive pancreatic progenitor cells are obtained by contacting PDX1-positive, NKX6.1-negative pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily, to induce the differentiation of at least some of the PDX1-positive, NKX6.1-negative pancreatic progenitor cells into PDX1-positive, NKX6.1-positive pancreatic progenitor cells. In some embodiments, following 3, 4, or 5 days of contacting the PDX1-positive, NKX6.1-positive pancreatic progenitor cells are obtained by contacting PDX1-positive, NKX6.1-negative pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily; the cells are then contacted with i) at least one growth factor from the FGF family, ii) at least one SHH pathway inhibitor, and optionally iii) a RA signaling pathway activator, iv) ROCK inhibitor, and v) at least one growth factor from the TGF-β superfamily, and vi) a PKC activator and optionally a gamma-secretase inhibitor. In some cases, the PDX1-positive, NKX6.1-positive pancreatic progenitor cells are obtained by contacting PDX1-positive, NKX6.1-negative pancreatic progenitor cells with at least one growth factor from the FGF family. In some cases, the growth factor from the FGF family is KGF.

Any growth factor from the FGF family capable of inducing PDX1-positive, NKX6.1-negative pancreatic progenitor cells to differentiate into PDX1-positive, NKX6.1-positive pancreatic progenitor cells (e.g., alone, or with any combination of at least one SHH pathway inhibitor, a ROCK inhibitor, a growth factor from the TGF-β superfamily, and at least one retinoic acid signaling pathway activator) can be used in the method provided herein. In some cases, the at least one growth factor from the FGF family comprises keratinocyte growth factor (KGF). In some cases, the at least one growth factor from the FGF family is selected from the group consisting of FGF8B, FGF10, and FGF21. In some examples, the method comprises contacting PDX1-positive, NKX6.1-negative pancreatic progenitor cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting PDX1-positive, NKX6.1-negative pancreatic progenitor cells with a concentration of a growth factor from FGF family (e.g., KGF), such as, 10-200 ng/mL, 10-150 ng/mL, 10-100 ng/mL, 25-75 ng/mL, 40-60 ng/mL, or 45-55 ng/mL.

Any SHH pathway inhibitor capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one retinoic acid signaling pathway activator, ROCK inhibitor, and at least one growth factor from the TGF-β superfamily) can be used in the method provided herein. In some cases, the SHH pathway inhibitor comprises Sant1. In some examples, the method comprises contacting PDX1-positive, NKX6.1-negative pancreatic progenitor cells with a concentration of a SHH pathway inhibitor (e.g., Sant1), such as, about 0.001 µM, about 0.002 µM, about 0.005 µM, about 0.01 µM, about 0.02 µM, about 0.03 µM, about 0.05 µM, about 0.08 µM, about 0.1 µM, about 0.12 µM, about 0.13 µM, about 0.14 µM, about 0.15 µM, about 0.16 µM, about 0.17 µM, about 0.18 µM, about 0.19 µM, about 0.2 µM, about 0.21 µM, about 0.22 µM, about 0.23 µM, about 0.24 µM, about 0.25 µM, about 0.26 µM, about 0.27 µM, about 0.28 µM, about 0.29 µM, about 0.3 µM, about 0.31 µM, about 0.32 µM, about 0.33 µM, about 0.34 µM, about 0.35 µM, about 0.4 µM, about 0.45 µM, about 0.5 µM, about 0.6 µM, about 0.8 µM, about 1 µM, about 2 µM, or about 5 µM. In some examples, the method comprises contacting PDX1-positive, NKX6.1-negative pancreatic progenitor cells with a concentration of a SHH pathway inhibitor (e.g., Sant1), such as, 50-1000 nM, 50-500 nM, 50-300 nM, 100-300 nM, 200-300 nM, 200-500 nM, or 225-275 nM.

Any RA signaling pathway activator capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some cases, the RA signaling pathway activator comprises retinoic acid. In some examples, the method comprises contacting PDX1-positive, NKX6.1-negative pancreatic progenitor cells with a concentration of an RA signaling pathway activator (e.g., retinoic acid), such as, about 0.02 µM, about 0.1 µM, about 0.2 µM, about 0.25 µM, about 0.3 µM, about 0.4 µM, about 0.45 µM, about 0.5 µM, about 0.55 µM, about 0.6 µM, about 0.65 µM, about 0.7 µM, about 0.75 µM, about 0.8 µM, about 0.85 µM, about 0.9 µM, about 1 µM, about 1.1 µM, about 1.2 µM, about 1.3 µM, about 1.4 µM, about 1.5 µM, about 1.6 µM, about 1.7 µM, about 1.8 µM, about 1.9 µM, about 2 µM, about 2.1 µM, about 2.2 µM, about 2.3 µM, about 2.4 µM, about 2.5 µM, about 2.6 µM, about 2.7 µM, about 2.8 µM, about 3 µM, about 3.2 µM, about 3.4 µM, about 3.6 µM, about 3.8 µM, about 4 µM, about 4.2 µM, about 4.4 µM, about 4.6 µM, about 4.8 µM, about 5 µM, about 5.5 µM, about 6 µM, about 6.5 µM, about 7 µM, about 7.5 µM, about 8 µM, about 8.5 µM, about 9 µM, about 9.5 µM, about 10 µM, about 12 µM, about 14 µM, about 15 µM, about 16 µM, about 18 µM, about 20 µM, about 50 µM, or about 100 µM. In some examples, the method comprises contacting PDX1-positive, NKX6.1-negative pancreatic progenitor cells with a concentration of an RA signaling pathway activator (e.g., retinoic acid), such as, 1-500 nM, 50-400 nM, 50-250 nM, 50-150 nM, 80-200 nM, 75-125 nM, or 90-110 nM.

Any ROCK inhibitor capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some cases, the ROCK inhibitor comprises Thiazovivin, Y-27632, Fasudil/HA1077, or 14-1152. In some examples, the method comprises contacting PDX1-positive, NKX6.1-negative pancreatic progenitor cells with a concentration of a ROCK inhibitor (e.g., Y-27632 or Thiazovivin), such as, about 0.2 µM, about 0.5 µM, about 0.75 µM, about 1 µM, about 2 µM, about 3 µM, about 4 µM, about 5 µM, about 6 µM, about 7 µM, about 7.5 µM, about 8 µM, about 9 µM, about 10 µM, about 11 µM, about 12 µM, about 13 µM, about 14 µM, about 15 µM, about 16 µM, about 17 µM, about 18 µM, about 19 µM, about 20 µM, about 21 µM, about 22 µM, about 23 µM, about 24 µM, about 25 µM, about 26 µM, about 27 µM, about 28 µM, about 29 µM, about 30 µM, about 35 µM, about 40 µM, about 50 µM, or about 100 µM. In some examples, the method comprises contacting PDX1-positive, NKX6.1-negative pancreatic progenitor cells with a concentration of a ROCK inhibitor (e.g., Y-27632 or Thiazovivin), such as, 0.2-5 µM, 0.8-3 µM, 1-4 µM, 1.5-4 µM, 1.8-3.5 µM, 2-3 µM, 2.4-2.6 µM.

Any activator from the TGF-β superfamily capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some cases, the activator from the TGF-β superfamily comprises Activin A or GDF8. In some examples, the method comprises contacting PDX1-positive, NKX6.1-negative pancreatic progenitor cells with a concentration of a growth factor from TGF-β superfamily (e.g., Activin A), such as, about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 1 ng/mL, about 1.2 ng/mL, about 1.4 ng/mL, about 1.6 ng/mL, about 1.8 ng/mL, about 2 ng/mL, about 2.2 ng/mL, about 2.4 ng/mL, about 2.6 ng/mL, about 2.8 ng/mL, about 3 ng/mL, about 3.2 ng/mL, about 3.4 ng/mL, about 3.6 ng/mL, about 3.8 ng/mL, about 4 ng/mL, about 4.2 ng/mL, about 4.4 ng/mL, about 4.6 ng/mL, about 4.8 ng/mL, about 5 ng/mL, about 5.2 ng/mL, about 5.4 ng/mL, about 5.6 ng/mL, about 5.8 ng/mL, about 6 ng/mL, about 6.2 ng/mL, about 6.4 ng/mL, about 6.6 ng/mL, about 6.8 ng/mL, about 7 ng/mL, about 8 ng/mL, about 9 ng/mL, about 10 ng/mL, about 20 ng/mL, about 30 ng/mL, or about 50 ng/mL. In some examples, the method comprises contacting PDX1-positive, NKX6.1-negative pancreatic progenitor cells with a concentration of a growth factor from TGF-β superfamily (e.g., Activin A), such as, about 5 ng/mL. In some embodiments, the concentration of the growth factor from TGF-β superfamily (e.g., Activin A) is 1-15 ng/mL, 3-12 ng/mL, 5-12 ng/mL, 5-20 ng/mL, 8-20 ng/mL, 8-15 ng/mL, 9-11 ng/mL, or 8-12 ng/mL.

In some cases, the PDX1-positive, NKX6.1-positive pancreatic progenitor cells are obtained by contacting PDX1-positive, NKX6.1-negative pancreatic progenitor cells with KGF, Sant1, and RA, for a period of 5 days or 6 days. In some cases, the PDX1-positive, NKX6.1-positive pancreatic progenitor cells are obtained by contacting PDX1-positive, NKX6.1-negative pancreatic progenitor cells with KGF, Sant1, RA, thiazovivin, and Activin A, for a period of 5 or 6 days. In some cases, the PDX1-positive, NKX6.1-positive pancreatic progenitor cells are obtained by contacting PDX1-positive, NKX6.1-negative pancreatic progenitor cells with KGF for a period of 5 days. In some cases, the PDX1-positive, NKX6.1-positive pancreatic progenitor cells are obtained by contacting PDX1-positive, NKX6.1-negative pancreatic progenitor cells with: i) at least one growth factor from the FGF family (e.g., KGF), ii) at least one SHH pathway inhibitor (e.g., Sant1), iii) at least one RA signaling pathway activator (e.g., retinoic acid), iv) at least one ROCK inhibitor (e.g., thiazovivin), and v) at least one growth factor from the TGF-β superfamily (e.g., activin A) for 1, 2, 3, 4, 5, 6, 7, 8 or 9 days (e.g., 5 or 6 days).

### Insulin-Positive Endocrine Cells

Aspects of the disclosure involve insulin-positive endocrine cells (e.g., NKX6.1-positive, ISL1-positive cells, or β-like cells). Insulin-positive endocrine cells of use herein can be derived from any source or generated in accordance with any suitable protocol, In some aspects, PDX-1 positive, NKX6.1-positive pancreatic progenitor cells are differentiated to insulin-positive endocrine cells (e.g., NKX6.1-positive, ISL1-positive cells, or β-like cells), In some aspects, the insulin-positive endocrine cells are further differentiated, e.g., by induction or maturation to SC-β cells.

In some aspects, a method of producing an insulin-positive endocrine cell from a PDX1-positive, NKX6.1-positive pancreatic progenitor cell comprises contacting a population of cells (e.g., under conditions that promote cell clustering) comprising PDX1-positive, NKX6-1-positive pancreatic progenitor cells with a) a TGF-β signaling pathway inhibitor, b) a thyroid hormone signaling pathway activator, c) a gamma-secretase inhibitor, and d) a protein kinase inhibitor to induce the differentiation of at least one PDX1-positive, NKX6.1-positive pancreatic progenitor cell in the population into an insulin-positive endocrine cell, wherein the insulin-positive endocrine ceil expresses insulin. In some cases, insulin-positive endocrine cells express PDX1, NKX6.1, ISL1, NKX2.2, Mafb, glis3, Sur1, Kir6.2, Znt8, SLC2A1, SLC2A3 and/or insulin.

Any TGF-β signaling pathway inhibitor capable of inducing the differentiation of PDX-1 positive, NKX6.1-positive pancreatic progenitor cells to differentiate into insulin-positive endocrine cells (e.g., alone, or in combination with other β cell-differentiation factors, e.g., a thyroid hormone signaling pathway activator) can be used. In some cases, the TGF-β signaling pathway comprises TGF-β receptor type I kinase signaling. In some cases, the TGF-β signaling pathway inhibitor comprises Alk5 inhibitor II.

Any thyroid hormone signaling pathway activator capable of inducing the differentiation of PDX-1 positive, NKX6.1-positive pancreatic progenitor cells to differentiate into insulin-positive endocrine cells (e.g., alone, or in combination with other β cell-differentiation factors, e.g., a TGF-β signaling pathway inhibitor) can be used. In some cases, the thyroid hormone signaling pathway activator comprises triiodothyronine (T3). In some cases, the thyroid hormone signaling pathway activator comprises GC-1.

In some cases, the method comprises contacting the population of cells (e.g., PDX-1 positive, NKX6.1-positive pancreatic progenitor cells) with at least one additional factor. In some cases, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) a protein kinase inhibitor, vi) a TGF-β signaling pathway inhibitor, or vii) a thyroid hormone signaling pathway activator. In some embodiments, the method comprises contacting the population of cells (e.g., PDX-1 positive, NKX6.1-positive pancreatic progenitor cells) with at least one additional factor. In some cases, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) a growth factor from the epidermal growth factor (EGF) family, v) a protein kinase inhibitor, vi) a TGF-β signaling pathway inhibitor, vii) a thyroid hormone signaling pathway activator, or viii) a PKC activator.

In some cases, the method comprises contacting the PDX1-positive, NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) a protein kinase inhibitor, or ix) a ROCK inhibitor.

In some cases, the method comprises contacting the PDX1-positive, NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, or x) a ROCK inhibitor.

In some cases, the method comprises contacting the PDX1-positive, NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, or x) a ROCK inhibitor.

In some embodiments, in the method of generating the insulin-positive endocrine cells from the PDX1-positive NKX6.1-postive pancreatic progenitor cells, some of the differentiation factors are present only for the first 1, 2, 3, 4, or 5 days during the differentiation step.

Any TGF-β signaling pathway inhibitor capable of inducing the differentiation of NKX6.1-positive pancreatic progenitor cells to differentiate into insulin-positive endocrine cells (e.g., alone, or in combination with other β cell-differentiation factors, e.g., a thyroid hormone signaling pathway activator) can be used. In some embodiments, the TGF-β signaling pathway comprises TGF-β receptor type I kinase signaling. In some embodiments, the TGF-β signaling pathway inhibitor comprises Alk5 inhibitor II. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 0.1 µM, about 0.5 µM, about 1 µM, about 1.5 µM, about 2 µM, about 2.5 µM, about 3 µM, about 3.5 µM, about 4 µM, about 4.5 µM, about 5 µM, about 5.5 µM, about 6 µM, about 6.5 µM, about 7 µM, about 7.5 µM, about 8 µM, about 8.5 µM, about 9 µM, about 9.5 µM, about 10 µM, about 10.5 µM, about 11 µM, about 11.5 µM, about 12 µM, about 12.5 µM, about 13 µM, about 13.5 µM, about 14 µM, about 14.5 µM, about 15 µM, about 15.5 µM, about 16 µM, about 16.5 µM, about 17 µM, about 17.5 µM, about 18 µM, about 18.5 µM, about 19 µM, about 19.5 µM, about 20 µM, about 25 µM, about 30 µM, about 35 µM, about 40 µM, about 45 µM, or about 50 µM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 7-13 µM, about 8-12 µM, about 9-11 µM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 10 µM.

Any thyroid hormone signaling pathway activator capable of inducing the differentiation of NKX6.1-positive pancreatic progenitor cells to differentiate into insulin-positive endocrine cells (e.g., alone, or in combination with other β cell-differentiation factors, e.g., a TGF-β signaling pathway inhibitor) can be used. In some embodiments, the thyroid hormone signaling pathway activator comprises triiodothyronine (T3). In some embodiments, the thyroid hormone signaling pathway activator comprises GC-1. In some examples, the method comprises contacting NKX6.1-positive pancreatic progenitor cells with a concentration of thyroid hormone signaling pathway activator (e.g., GC-1), such as, about 0.1 µM, about 0.12 µM, about 0.13 µM, about 0.14 µM, about 0.15 µM, about 0.16 µM, about 0.17 µM, about 0.18 µM, about 0.19 µM, about 0.2 µM, about 0.21 µM, about 0.22 µM, about 0.23 µM, about 0.24 µM, about 0.25 µM, about 0.26 µM, about 0.27 µM, about 0.28 µM, about 0.29 µM, about 0.3 µM, about 0.31 µM, about 0.32 µM, about 0.33 µM, about 0.34 µM, about 0.35 µM, about 0.4 µM, about 0.45 µM, about 0.5 µM, about 0.6 µM, about 0.8 µM, about 1 µM, about 2 µM, or about 5 µM. In some examples, the method comprises contacting NKX6.1-positive pancreatic progenitor cells with a concentration of thyroid hormone signaling pathway activator (e.g., GC-1), such as, about 0.7-1.3 µM, about 0.8-1.2 µM, or about 0.9-1.1 µM. In some examples, the method comprises contacting NKX6.1-positive pancreatic progenitor cells with a concentration of thyroid hormone signaling pathway activator (e.g., GC-1), such as, about 1 µM.

Any γ-secretase inhibitor that is capable of inducing the differentiation of PDX-1 positive, NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the γ-secretase inhibitor comprises XXI. In some cases, the γ-secretase inhibitor comprises DAPT. In some examples, the method comprises contacting PDX-1 positive, NKX6.1-positive pancreatic progenitor cells with a concentration of a γ-secretase inhibitor (e.g., XXI), such as, about 0.01 µM, about 0.02 µM, about 0.05 µM, about 0.075 µM, about 0.1 µM, about 0.2 µM, about 0.3 µM, about 0.4 µM, about 0.5 µM, about 0.6 µM, about 0.7 µM, about 0.8 µM, about 0.9 µM, about 1 µM, about 1.1 µM, about 1.2 µM, about 1.3 µM, about 1.4 µM, about 1.5 µM, about 1.6 µM, about 1.7 µM, about 1.8 µM, about 1.9 µM, about 2 µM, about 2.1 µM, about 2.2 µM, about 2.3 µM, about 2.4 µM, about 2.5 µM, about 2.6 µM, about 2.7 µM, about 2.8 µM, about 2.9 µM, about 3 µM, about 3.2 µM, about 3.4 µM, about 3.6 µM, about 3.8 µM, about 4 µM, about 4.2 µM, about 4.4 µM, about 4.6 µM, about 4.8 µM, about 5 µM, about 5.2 µM, about 5.4 µM, about 5.6 µM, about 5.8 µM, about 6 µM, about 6.2 µM, about 6.4 µM, about 6.6 µM, about 6.8 µM, about 7 µM, about 8 µM, about 9 µM, about 10 µM, about 20 µM, about 30 µM, or about 50 µM. In some examples, the method comprises contacting NKX6.1-positive pancreatic progenitor cells with a concentration of a γ-secretase inhibitor (e.g., XXI), such as, about 1.7-2.3 µM, about 1.8-2.2 µM, or about 1.9-2.1 µM. In some examples, the method comprises contacting NKX6.1-positive pancreatic progenitor cells with a concentration of a γ-secretase inhibitor (e.g., XXI), such as about 2 µM.

Any growth factor from the EGF family capable of inducing the differentiation of PDX-1 positive, NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some cases, the at least one growth factor from the EG F family comprises betacellulin. In some cases, at least one growth factor from the EGF family comprises EGF. In some examples, the method comprises contacting PDX-1 positive, NKX6.1-positive pancreatic progenitor cells with a concentration of a growth factor from EGF family (e.g., betacellulin), such as, about 1 ng/mL, about 2 ng/mL, about 4 ng/mL, about 6 ng/mL, about 8 ng/mL, about 10 ng/mL, about 12 ng/mL, about 14 ng/mL, about 16 ng/mL, about 18 ng/mL, about 20 ng/mL, about 22 ng/mL, about 24 ng/mL, about 26 ng/mL, about 28 ng/mL, about 30 ng/mL, about 40 ng/mL, about 50 ng/mL, about 75 ng/mL, about 80 ng/mL, about 90 ng/mL, about 95 ng/mL, about 100 ng/mL, about 150 ng/mL, about 200 ng/mL, about 250 ng/mL, or about 300 ng/mL. In some examples, the method comprises contacting NKX6.1-positive pancreatic progenitor cells with a concentration of a growth factor from EGF family (e.g., betacellulin), such as, about 17-23 ng/ml, about 18-22 ng/ml, or about 19-21 ng/ml. In some examples, the method comprises contacting NKX6.1-positive pancreatic progenitor cells with a concentration of a growth factor from EGF family (e.g., betacellulin), such as, about 20 ng/ml.

Any RA signaling pathway activator capable of inducing the differentiation of PDX-1 positive, NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the RA signaling pathway activator comprises RA. In some examples, the method comprises contacting PDX-1 positive, NKX6.1-positive pancreatic progenitor cells with a concentration of an RA signaling pathway activator (e.g., retinoic acid), such as, about 0.02 µM, about 0.1 µM, about 0.2 µM, about 0.25 µM, about 0.3 µM, about 0.4 µM, about 0.45 µM, about 0.5 µM, about 0.55 µM, about 0.6 µM, about 0.65 µM, about 0.7 µM, about 0.75 µM, about 0.8 µM, about 0.85 µM, about 0.9 µM, about 1 µM, about 1.1 µM, about 1.2 µM, about 1.3 µM, about 1.4 µM, about 1.5 µM, about 1.6 µM, about 1.7 µM, about 1.8 µM, about 1.9 µM, about 2 µM, about 2.1 µM, about 2.2 µM, about 2.3 µM, about 2.4 µM, about 2.5 µM, about 2.6 µM, about 2.7 µM, about 2.8 µM, about 3 µM, about 3.2 µM, about 3.4 µM, about 3.6 µM, about 3.8 µM, about 4 µM, about 4.2 µM, about 4.4 µM, about 4.6 µM, about 4.8 µM, about 5 µM, about 5.5 µM, about 6 µM, about 6.5 µM, about 7 µM, about 7.5 µM, about 8 µM, about 8.5 µM, about 9 µM, about 9.5 µM, about 10 µM, about 12 µM, about 14 µM, about 15 µM, about 16 µM, about 18 µM, about 20 µM, about 50 µM, or about 100 µM. In some examples, the method comprises contacting NKX6.1-positive pancreatic progenitor cells with a concentration of an RA signaling pathway activator (e.g., retinoic acid), such as, about 20-80 nM, about 30-70 nM, or about 40-60 nM. In some examples, the method comprises contacting NKX6.1-positive pancreatic progenitor cells with a concentration of an RA signaling pathway activator (e.g., retinoic acid), such as, about 50 nM.

Any SHH pathway inhibitor capable of inducing the differentiation of PDX-1 positive, NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used in the method provided herein. In some cases, the SHH pathway inhibitor comprises Sant1. In some examples, the method comprises contacting PDX-1 positive, NKX6.1-positive pancreatic progenitor cells with a concentration of a SHH pathway inhibitor (e.g., Sant1), such as, about 0.001 µM, about 0.002 µM, about 0.005 µM, about 0.01 µM, about 0.02 µM, about 0.03 µM, about 0.05 µM, about 0.08 µM, about 0.1 µM, about 0.12 µM, about 0.13 µM, about 0.14 µM, about 0.15 µM, about 0.16 µM, about 0.17 µM, about 0.18 µM, about 0.19 µM, about 0.2 µM, about 0.21 µM, about 0.22 µM, about 0.23 µM, about 0.24 µM, about 0.25 µM, about 0.26 µM, about 0.27 µM, about 0.28 µM, about 0.29 µM, about 0.3 µM, about 0.31 µM, about 0.32 µM, about 0.33 µM, about 0.34 µM, about 0.35 µM, about 0.4 µM, about 0.45 µM, about 0.5 µM, about 0.6 µM, about 0.8 µM, about 1 µM, about 2 µM, or about 5 µM. In some examples, the method comprises contacting NKX6.1-positive pancreatic progenitor cells with a concentration of a SHH pathway inhibitor (e.g., Sant1), such as, about 220-280 nM, about 230-270 nM, about 240-260 nM, or about 245-255 nM. In some examples, the method comprises contacting NKX6.1-positive pancreatic progenitor cells with a concentration of a SHH pathway inhibitor (e.g., Sant1), such as, about 250 nM.

Any BMP signaling pathway inhibitor capable of inducing the differentiation of PDX-1 positive, NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some cases, the BMP signaling pathway inhibitor comprises LDN193189 or DMH-1. In some examples, the method comprises contacting PDX-1 positive, NKX6.1-positive pancreatic progenitor cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 µM. In some examples, the method comprises contacting NKX6.1-positive pancreatic progenitor cells with a concentration of BMP signaling pathway inhibitor (e.g., LDN193189), such as, about 70-130 nM, about 80-120 nM, about 90-110 nM. In some examples, the method comprises contacting NKX6.1-positive pancreatic progenitor cells with a concentration of BMP signaling pathway inhibitor (e.g., LDN193189), such as, about 100 nM.

Any ROCK inhibitor that is capable of inducing the differentiation of PDX-1 positive, NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some cases, the ROCK inhibitor comprises Thiazovivin, Y-27632, Fasudil/HA1077, or H-1152. In some cases, the ROCK inhibitor comprises Y-27632. In some cases, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 0.2 µM, about 0.5 µM, about 0.75 µM, about 1 µM, about 2 µM, about 3 µM, about 4 µM, about 5 µM, about 6 µM, about 7 µM, about 7.5 µM, about 8 µM, about 9 µM, about 10 µM, about 11 µM, about 12 µM, about 13 µM, about 14 µM, about 15 µM, about 16 µM, about 17 µM, about 18 µM, about 19 µM, about 20 µM, about 21 µM, about 22 µM, about 23 µM, about 24 µM, about 25 µM, about 26 µM, about 27 µM, about 28 µM, about 29 µM, about 30 µM, about 35 µM, about 40 µM, about 50 µM, or about 100 µM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.2-2.8 µM, about 2.3-2.7 µM, or about 2.4-2.6 µM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.5 µM.

Any epigenetic modifying compound that is capable of inducing the differentiation of PDX-1 positive, NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some cases, the epigenetic modifying compound comprises a histone methyltransferase inhibitor or a HDAC inhibitor. In some cases, the epigenetic modifying compound comprises a histone methyltransferase inhibitor, e.g., DZNep. In some cases, the epigenetic modifying compound comprises a HDAC inhibitor, e.g., KD5170. In some examples, the method comprises contacting PDX1-positive, NKX6.1-positive pancreatic progenitor cells with a concentration of an epigenetic modifying compound (e.g., DZNep or KD5170), such as, about 0.01 µM, about 0.025 µM, about 0.05 µM, about 0.075 µM, about 0.1 µM, about 0.15 µM, about 0.2 µM, about 0.5 µM, about 0.75 µM, about 1 µM, about 2 µM, about 3 µM, about 4 µM, about 5 µM, about 6 µM, about 7 µM, about 7.5 µM, about 8 µM, about 9 µM, about 10 µM, about 15 µM, about 20 µM, about 25 µM, about 30 µM, about 35 µM, about 40 µM, about 50 µM, or about 100 µM. In some examples, the method comprises contacting PDX1-positive, NKX6.1-positive pancreatic progenitor cells with a concentration of an epigenetic modifying compound (e.g., DZNep or KD5170), such as, about 70-130 nM, about 80-120 nM, or about 90-110 nM. In some examples, the method comprises contacting PDX1-positive, NKX6.1-positive pancreatic progenitor cells with a concentration of an epigenetic modifying compound (e.g., DZNep or KD5170), such as, about 100 nM.

In some cases, the population of cells is optionally contacted with a protein kinase inhibitor. In some cases, the population of cells is not contacted with the protein kinase inhibitor. In some cases, the population of cells is contacted with the protein kinase inhibitor. Any protein kinase inhibitor that is capable of inducing the differentiation of PDX-1 positive, NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some cases, the protein kinase inhibitor comprises staurosporine. In some examples, the method comprises contacting NKX6.1-positive pancreatic progenitor cells with a concentration of a protein kinase inhibitor (e.g., staurosporine), such as, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, about 2.0 nM, about 2.1 nM, about 2.2 nM, about 2.3 nM, about 2.4 nM, about 2.5 nM, about 2.6 nM, about 2.7 nM, about 2.8 µM, about 2.9 nM, about 3 nM, about 3.1 nM, about 3.2 nM, about 3.3 nM, about 3.4 nM, about 3.5 nM, about 3.6 nM, about 3.7 nM, about 3.8 nM, about 3.9 nM, about 4.0 nM, about 4.1 nM, about 4.2 nM, about 4.3 nM, about 4.4 nM, about 4.5 nM, about 4.6 nM, about 4.7 nM, about 4.8 µM, about 4.9 nM, or about 5 nM. In some examples, the method comprises contacting NKX6.1-positive pancreatic progenitor cells with a concentration of a protein kinase inhibitor (e.g., staurosporine), such as, about 1-5 nM, about 2-4 nM, or about 2.5-3.5 nM. In some examples, the method comprises contacting NKX6.1-positive pancreatic progenitor cells with a concentration of a protein kinase inhibitor (e.g., staurosporine), such as, about 3 nM.

In some cases, the method comprises contacting the population of cells (e.g., PDX-1 positive, NKX6.1-positive pancreatic progenitor cells) with XXI, Alk5i, T3 or GC-1, RA, Sant1, and betacellulin for a period of 7 days, to induce the differentiation of at least one PDX-1 positive, NKX6.1-positive pancreatic progenitor cell in the population into an insulin-positive endocrine cell, wherein the insulin-positive endocrine cell expresses insulin. In some cases, the method comprises contacting the population of cells (e.g., PDX-1 positive, NKX6.1-positive pancreatic progenitor cells) with XXI, Alk5i, T3 or GC-1, RA, Sant1, betacellulin, and LDN193189 for a period of 7 days, to induce the differentiation of at least one PDX-1 positive, NKX6.1-positive pancreatic progenitor cell in the population into an insulin-positive endocrine cell, wherein the insulin-positive endocrine ceil expresses insulin. In some embodiments, one or more differentiation factors are added in a portion of the Stage 5, for instance, only the first 1, 2, 3, 4, 5, or 6 days of the period of time for Stage 5, or the last 1, 2, 3, 4, 5, or 6 days of the period of time for Stage 5. In one example, the cells are contacted with SHH signaling pathway inhibitor for only the first 2, 3, 4, or 5 days during Stage 5, after which the SHH signaling pathway inhibitor is removed from the culture medium. In another example, the cells are contacted with BMP signaling pathway inhibitor for only the first 1, 2, or 3 days during Stage 5, after which the BMP signaling pathway inhibitor is removed from the culture medium. In some embodiments, PDX-1 positive, NKX6.1-positive pancreatic progenitor cells are contacted with i) a SHH pathway inhibitor (e.g., Sant1), ii) a RA signaling pathway activator (e.g., retinoic acid), iii) a γ-secretase inhibitor (e.g., XXI), iv) a growth factor from the epidermal growth factor (EGF) family (e.g., betacellulin), v) at least one bone morphogenetic protein (BMP) signaling pathway inhibitor (e.g., LDN-193189), vi) a TGF-β signaling pathway inhibitor (e.g., Alk5i), vii) a thyroid hormone signaling pathway activator (e.g., GC-1), viii) an epigenetic modifying compound (e.g., DZNEP), ix) a protein kinase inhibitor (e.g., staurosporine), and/or x) a ROCK inhibitor (e.g., thiazovivin) for 1, 2, 3, 4, 5, or 6 days (e.g., 2 or 3 days); and the cells are then contacted with i) a γ-secretase inhibitor (e.g., XXI), ii) at least one bone morphogenetic protein (BMP) signaling pathway inhibitor (e.g., LDN-193189), iii) a TGF-β signaling pathway inhibitor (e.g., Alk5i), iv) a thyroid hormone signaling pathway activator (e.g., GC-1), v) an epigenetic modifying compound (e.g., DZNEP), vi) a protein kinase inhibitor (e.g., staurosporine), and/or vii) a ROCK inhibitor (e.g., thiazovivin) for 1, 2, 3, 4, 5, 6 or 7 days (e.g., 4 or 5 days) in the absence of i) a SHH pathway inhibitor (e.g., Sant1), ii) a RA signaling pathway activator (e.g., retinoic acid), and iii) a growth factor from the epidermal growth factor (EGF) family (e.g., betacellulin) .

In some cases, the method comprises culturing the population of cells (e.g., PDX-1 positive, NKX6.1-positive pancreatic progenitor cells) in a BE5 medium, to induce the differentiation of at least one PDX-1 positive, NKX6.1-positive pancreatic progenitor cell in the population into an insulin-positive endocrine cell, wherein the insulin-positive endocrine cell expresses insulin.

Aspects of the disclosure involve treatment of cell population comprising PDX1-positive, NKX6.1-positive pancreatic progenitor cells with PKC activator, which may lead to increase in percentage of pancreatic α cells, increase in percentage of pancreatic δ cells, increase in percentage of pancreatic β cells, reduction in percentage of EC cells, or any combination thereof, in the cell population of pancreatic endocrine cells generated according to the method disclosed herein.

In some cases, the method comprises contacting a population of cells comprising PDX1-positive, NKX6.1-positive pancreatic progenitor cells with a first composition comprising the PKC activator, a ROCK inhibitor, a growth factor from TGFβ superfamily, a growth factor from FGF family, a RA signaling pathway activator, and a SHH pathway inhibitor, for one to two days, thereby obtaining a first transformation cell population comprising PDX1-positive, NKX6.1-positive pancreatic progenitor cells; and contacting the first transformation cell population comprising PDX1-positive, NKX6.1-positive pancreatic progenitor cells with a second composition comprising the PKC activator, a TGF-β signaling pathway inhibitor, a TH signaling pathway activator, and an epigenetic modifying compound, for one to two days, thereby obtaining a second transformation cell population comprising NKX6.1-positive, ISL1-positive endocrine cells.

In some cases, the method comprises (1) contacting PDX1-positive, NKX6.1-negative pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily, for about two to six days, to induce the differentiation of at least some of the PDX1-positive, NKX6.1-negative pancreatic progenitor cells into PDX1-positive, NKX6.1-positive pancreatic progenitor cells; and (2) after (1) contacting the population comprising the PDX1-positive, NKX6.1-positive pancreatic progenitor cells with i) at least one growth factor from the FGF family, ii) at least one SHH pathway inhibitor, iii) a RA signaling pathway activator, iv) ROCK inhibitor, v) at least one growth factor from the TGF-β superfamily, and vi) a PKC activator, for one to two days, thereby generating a first transformation cell population comprising PDX1-positive, NKX6.1-positive pancreatic progenitor cells.

In some cases, the method further comprises: (3) contacting the first transformation cell population comprising PDX1-positive, NKX6.1-positive pancreatic progenitor cells with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, x) a ROCK inhibitor, and xi) a PKC activator, for one to two days, thereby generating a second transformation cell population; and (4) contacting the second transformation cell population with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, and x) a ROCK inhibitor, thereby generating a cell population comprising NKX6.1-positive, ISL1-positive endocrine cells.

### Pancreatic Β Cells

Aspects of the disclosure involve generating pancreatic β cells (e.g., non-native pancreatic β cells). Non-native pancreatic β cells, in some cases, resemble endogenous mature β cells in form and function, but nevertheless are distinct from native β cells.

In some cases, the insulin-positive pancreatic endocrine cells generated using the method provided herein can form a cell cluster, alone or together with other types of cells, e.g., precursors thereof, e.g., stem cell, definitive endoderm cells, primitive gut tube cell, PDX1-positive, NKX6.1-negative pancreatic progenitor cells, or NKX6.1-positive pancreatic progenitor cells.

In some cases, the cell population comprising the insulin-positive endocrine cells can be directly induced to mature into SC-β cells without addition of any exogenous differentiation factors (such as inhibitor of TGF-β signaling pathway, thyroid hormone signaling pathway activator, PKC activator, growth factors from TGF-β superfamily, FGF family, or EGF family, SHH signaling pathway inhibitor, γ-secretase inhibitor, ROCK inhibitor, or BMP signaling pathway inhibitor). In some embodiments, the method provided herein comprises contacting a cell population comprising NKX6.1-positive, ISL1-positive endocrine cells with a serum albumin protein, a TGF-β signaling pathway inhibitor, a SHH pathway inhibitor, a thyroid hormone signaling pathway activator, a protein kinase inhibitor, a ROCK inhibitor, a BMP signaling pathway inhibitor, and/or an epigenetic modifying compound. In some embodiments, the method provided herein comprises contacting a cell population comprising NKX6.1-positive, ISL1-positive endocrine cells with human serum albumin protein. In some embodiments, the method provided herein comprises contacting a cell population comprising NKX6.1-positive, ISL1-positive endocrine cells with a PKC activator.

In some examples, insulin-positive endocrine cells can be matured in a NS-GFs medium, MCDB131 medium, DMEM medium, or CMRL medium. In some cases, the insulin-positive endocrine cells can be matured in a CMRLs medium supplemented with 10% FBS. In some cases, the insulin-positive endocrine cells can be matured in a DMEM/F12 medium supplemented with 0.01-1% HSA (e.g., 0.05% HSA). In other cases, SC-β cells can be obtained by culturing the population of cells containing the insulin-positive endocrine cells in a MCDB 131 medium that can be supplemented by 2% BSA. In some cases, the MCDB131 medium with 2% BSA for maturation of insulin-positive endocrine cells into SC-β cells can be comprise no small molecule factors as described herein. In some case, the MCDB131 medium with 2% BSA for maturation of insulin-positive endocrine cells into SC-β cells can comprise no serum (e.g., no FBS). In other cases, SC-β cells can be obtained by culturing the population of cells containing the insulin-positive endocrine cells in a MCDB131 medium that can be supplemented by 0.05% HSA and vitamin C. In some embodiments, the cells are not contacted with vitamin C. In some cases, SC-β cells can be obtained by culturing the population of cells containing the insulin-positive endocrine cells in a MCDB 131 medium that can be supplemented by 0.05% HSA, ITS-X, vitamin C, and glutamine (Gln, e.g., 4 mM). In some cases, the type of culture medium may be changed during S6. For instance, the S6 cells are cultured in a MCDB131 medium that can be supplemented by 0.05% HSA and vitamin C for the first two to four days, and then followed by a DMEM/F12 medium supplemented with 1% HSA. In some cases, additional factors are introduced into the culture medium. For instance, S6 cells can be cultured in a MCDB 131 medium that can be supplemented by 0.05% HSA, ITS-X, vitamin C, and glutamine (Gln, e.g., 4 mM) throughout the 10-12 days, during which ZnSO₄ is introduced from day 4 of S6.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days ; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor, ii) a TH signaling pathway activator, iii) at least one SHH pathway inhibitor, iv) a RA signaling pathway activator, v) a γ-secretase inhibitor, optionally vi) at least one growth factor from the epidermal growth factor (EGF) family, and optionally vii) a BMP signaling pathway inhibitor, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium) without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells. In preferred embodiments, the cells for use in any of the methods disclosed herein are not cadaveric islet cells. In some embodiments, the compositions for use in any of the methods disclosed herein comprise at least 80%, 85%, 90%, 91%, 92%, 93%, 94%, 95%, 96%, 97%, 98%, 99% or 100% living cells (i.e., not dead or dying cells).

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor, v) a PKC activator, vi) a ROCK inhibitor, and vii) a growth factor from TGFβ superfamily, for a period of 2 days; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor, ii) a TH signaling pathway activator, iii) at least one SHH pathway inhibitor, iv) a RA signaling pathway activator, v) a γ-secretase inhibitor, optionally vi) at least one growth factor from the epidermal growth factor (EGF) family, and optionally vii) a BMP signaling pathway inhibitor, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium )without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cell.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a PKC activator, and v) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor, ii) a TH signaling pathway activator, iii) at least one SHH pathway inhibitor, iv) a RA signaling pathway activator, v) a γ-secretase inhibitor, and optionally vi) at least one growth factor from the epidermal growth factor (EGF) family, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium), for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i)retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 or 6 days ; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound (e.g., DZNep or KD5170), ix) a protein kinase inhibitor, and x) a ROCK inhibitor, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., medium supplemented with HSA, NS-GFs medium, MCDB medium supplemented with BSA, MCDB131 medium, or DMEM/F12 medium), for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i)retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 or 6 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a γ-secretase inhibitor, ii) at least one bone morphogenetic protein (BMP) signaling pathway inhibitor, iii) a TGF-β signaling pathway inhibitor, iv) a thyroid hormone signaling pathway activator, v) an epigenetic modifying compound (e.g., DZNep or KD5170), vi) a protein kinase inhibitor, and vii) a ROCK inhibitor, for a period of between five and seven days, and within first three days of the period of between five and seven days, contacting the PDX1-positive, NKX6.1-positive pancreatic progenitor cells with a SHH pathway inhibitor, a RA signaling pathway, and at least one growth factor from the EGF family, which are removed from the PDX1-positive, NKX6.1-positive pancreatic progenitor cells thereafter; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., medium with HSA, NS-GFs medium, MCDB medium supplemented with BSA, MCDB131 medium, or DMEM/F12 medium), for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i)retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 3 or 4 days, followed by contacting with i) at least one growth factor from the FGF family, ii) at least one SHH pathway inhibitor, and optionally iii) a RA signaling pathway activator, and optionally iv) ROCK inhibitor, v) at least one factor from TGFβ superfamily, and vi) a PKC activator, and optionally vii) a gamma secretase inhibitor, for 1 to 2 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound (e.g., DZNep or KD5170), ix) a protein kinase inhibitor, x) a ROCK inhibitor, and xi) a PKC activator, for 1 to 2 days, followed by contacting with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound (e.g., DZNep or KD5170), ix) a protein kinase inhibitor, and x) a ROCK inhibitor, for a period of between three and six days; and f) differentiating at least some of the PDX1-positive, NKX6.1-positive, insulin-positive endocrine cells into SC-β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for one day followed by contacting the cells with the factor from the TGFβ superfamily for two additional days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a PKC activator, v) a ROCK inhibitor, and a BMP pathway inhibitor for 1 day, followed by contacting the cells with i) retinoic acid signaling pathway activator, ii) at least one factor from the FGF family, iii) a SHH pathway inhibitor, iv) a PKC activator, v) a ROCK inhibitor for another day; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor (e.g., Alk5i), ii) a TH signaling pathway activator (e.g., GC-1), iii) at least one SHH pathway inhibitor (e.g., Sant1), iv) a RA signaling pathway activator (e.g., retinoic acid), v) a γ-secretase inhibitor (e.g., XXI), vi) at least one growth factor from the epidermal growth factor (EGF) family (e.g., betacellulin), vii) a BMP pathway inhibitor (e.g., LDN193189), viii) a ROCK inhibitor (e.g., thiazovivin), ix) a protein kinase inhibitor (e.g., staurosporine), and x) an epigenetic modifier (e.g., DZNEP) for 2 or 3 days, followed by contacting the PDX1-positive, NKX6.1-positive pancreatic progenitor cells with i) a TGF-β signaling pathway inhibitor (e.g., Alk5i), ii) a TH signaling pathway activator (e.g., GC-1), iii) a γ-secretase inhibitor (e.g., XXI), iv) a BMP pathway inhibitor (e.g., LDN193189), v) a ROCK inhibitor (e.g., thiazovivin), vi) a protein kinase inhibitor (e.g., staurosporine), and vii) an epigenetic modifier (e.g., DZNEP), for a period of four or five days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., a medium comprising HSA), for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

The medium used to culture the cells dissociated from the first cell cluster can be xeno-free. A xeno-free medium for culturing cells and/or cell clusters of originated from an animal can have no product from other animals. In some cases, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some cases, medium can comprise about 10% of serum. In some cases, the medium can be free of small molecules and/or FBS. For example, a medium can comprise MCDB131 basal medium supplemented with 2% BSA. In some cases, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate 13-acetate, bryostatin 1, or derivatives thereof), antagonist of TGF β super family (e.g., A1k5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenetic Protein (BMP) type 1 receptor (e.g., LDN193189 or derivatives thereof), thyroid hormone signaling pathway activator (e.g., T3, GC-1 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some cases, the reaggregation medium can comprise no exogenous extracellular matrix molecule. In some cases, the reaggregation medium does not comprise Matrigel™. In some cases, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that that the concentration of serum albumin supplemented into the medium may vary. For example, a medium (e.g., MCDB131) can comprise about 0.01%, 0.05%, 0.1%, 1%, about 2%, about 3%, about 4%, about 5%, about 10%, or about 15% BSA. In other cases, a medium can comprise about 0.01%, 0.05%, 0.1%, 1%, about 2%, about 3%, about 4%, about 5%, about 10%, or about 15% HSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine. The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some cases, the medium comprises laminin, such as LN-332. In some cases, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

In some cases, cells can be cultured under dynamic conditions (e.g., under conditions in which the cells are subject to constant movement or stirring while in the suspension culture). For dynamic culturing of cells, the cells can be cultured in a container (e.g., an non-adhesive container such as a spinner flask (e.g., of 200 ml to 3000 ml, for example 250 ml; of 100 ml; or in 125 ml Erlenmeyer), which can be connected to a control unit and thus present a controlled culturing system. In some cases, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

Medium in a dynamic cell culture vessel (e.g., a spinner flask) can be stirred (e.g., by a stirrer). The spinning speed can correlate with the size of the re-aggregated second cell cluster. The spinning speed can be controlled so that the size of the second cell cluster can be similar to an endogenous pancreatic islet. In some cases, the spinning speed is controlled so that the size of the second cell cluster can be from about 75 µm to about 250 µm. The spinning speed of a dynamic cell culture vessel (e.g., a spinner flask) can be about 20 rounds per minute (rpm) to about 100 rpm, e.g., from about 30 rpm to about 90 rpm, from about 40 rpm to about 60 rpm, from about 45 rpm to about 50 rpm. In some cases, the spinning speed can be about 50 rpm.

Stage 6 cells as provided herein may or may not be subject to the dissociation and reaggregation process as described herein. In some cases, the cell cluster comprising the insulin-positive endocrine cells can be reaggregated. The reaggregation of the cell cluster can enrich the insulin-positive endocrine cells. In some cases, the insulin-positive endocrine cells in the cell cluster can be further matured into pancreatic β cells. For example, after reaggregation, the second cell cluster can exhibit in vitro GSIS, resembling native pancreatic islet. For example, after reaggregation, the second cell cluster can comprise non-native pancreatic β cell that exhibits in vitro GSIS. In some embodiments, the reaggregation process can be performed according to the disclosure of US20200332262, which is incorporated herein by reference in its entirety.

Stage 6 cells obtained according to methods provided herein can have high recovery yield after cryopreservation and reaggregation procedures. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without such treatment. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without treatment of a BMP signaling pathway inhibitor (e.g., DMH-1 or LDN) and a growth factor from TGF-β superfamily (e.g., Activin A) at stage 3. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a recovery yield after cryopreservation post stage 5 that is at least about 35%, 37.5%, 40%, 42.5%, 45%, 47.5%, 48%, 49%, or 50%. The recovery yield can be calculated as a percentage of cells that survive and form reaggregated cell clusters after cryopreservation, thawing and recovery, and reaggregation procedures, as compared to the cells before the cryopreservation.

In some embodiments, the present disclosure relates to cryopreservation of the non-native pancreatic β cells or precursors thereof (e.g., NKX6.1-positive, PDX1-positive, insulin-positive cells obtained following stage 5) obtained using the methods provided herein. In some embodiments, the cell population comprising non-native pancreatic β cells can be stored via cryopreservation. For instances, the cell population comprising non-native β cells, e.g., Stage 6 cells in some cases, can be dissociated into cell suspension, e.g., single cell suspension, and the cell suspension can be cryopreserved, e.g., frozen in a cryopreservation solution. The dissociation of the cells can be conducted by any of the technique provided herein, for example, by enzymatic treatment. The cells can be frozen at a temperature of at highest -20° C., at highest -30° C., at highest -40° C., at highest -50° C., at highest -60° C., at highest -70° C., at highest -80° C., at highest -90° C., at highest -100° C., at highest -110° C., at highest -120° C., at highest -130° C., at highest -140° C., at highest -150° C., at highest -160° C., at highest -170° C., at highest -180° C., at highest -190° C., or at highest -200° C. In some cases, the cells are frozen at a temperature of about -80° C. In some cases, the cells are frozen at a temperature of about -195° C. Any cooling methods can be used for providing the low temperature needed for cryopreservation, such as, but not limited to, electric freezer, solid carbon dioxide, and liquid nitrogen. In some cases, any cryopreservation solution available to one skilled in the art can be used for incubating the cells for storage at low temperature, including both custom made and commercial solutions. For example, a solution containing a cryoprotectant can be used. The cryoprotectant can be an agent that is configured to protect the cell from freezing damage. For instance, a cryoprotectant can be a substance that can lower the glass transition temperature of the cryopreservation solution. Exemplary cryoprotectants that can be used include DMSO (dimethyl sulfoxide), glycols (e.g., ethylene glycol, propylene glycol and glycerol), dextran (e.g., dextran-40), and trehalose. Additional agents can be added in to the cryopreservation solution for other effects. In some cases, commercially available cryopreservation solutions can be used in the method provided herein, for instance, FrostaLife™, pZerve™, Prime-XV®, Gibco Synth-a-Freeze Cryopreservation Medium, STEM-CELLBANKER®, CryoStor® Freezing Media, HypoThermosol® FRS Preservation Media, and CryoDefend® Stem Cells Media.

During the differentiation process, the cells can be subject to irradiation treatment as provided herein. In particular embodiments, the cells are not subject to irradiation treatment. In some cases, the cell population at Stage 6, e.g., the cell population or cell cluster that has cells being differentiated from insulin-positive endocrine cells into pancreatic β cells, is irradiated for a period of time. In some cases, the cell population at Stage 6 after reaggregation following the recovery from cryopreservation is irradiated for a period of time. In some cases, the cryopreserved cells (e.g., the cells that are cryopreserved at the end of Stage 5) are irradiated for a certain period of time prior to thawing and recovery for subsequent differentiation process.

In some embodiments, the stage 6 cells comprise NKX6.1-positive, insulin-positive cells. In some embodiments, the stage 6 cells comprise NKX6.1-positive, insulin-negative cells. In some embodiments, the stage 6 cells comprise C-peptide positive cells. In some embodiments, Stage 6 cells or cells that have characteristics of stage 6 cells are incubated in NS-GFs medium, MCDB131 medium, DMEM medium, or CMRL medium. In some embodiments, the stage 6 cells or cells that have characteristics of stage 6 cells are contacted with any one or more of a vitamin or anti-oxidant (e.g., vitamin C), an albumin protein (e.g., a human serum albumin protein), a TGF-beta pathway inhibitor (e.g., an ALK5 inhibitor II), a bone morphogenic protein (BMP) type 1 receptor inhibitor (e.g., LDN193189), a Rho-associated coiled-coil containing protein kinase (ROCK) inhibitor (e.g., thiazovivin), a histone methyltransferase inhibitor (e.g., DZNEP), and/or a protein kinase inhibitor (e.g., staurosporine). In some embodiments, the stage 6 cells are contacted with a PKC activator (see, e.g., US20210214690A1, which is incorporated by reference herein in its entirety). In some embodiments, the stage 6 cells are not contacted with a PKC activator.

In some embodiments, the disclosure provides for a composition comprising a population of insulin-positive cells and a lipid. In some embodiments, the disclosure provides for a method of contacting a population of insulin-positive cells with a lipid. In some embodiments, the lipid is a saturated fatty acid. In some embodiments, the saturated fatty acid is palmitate. In some embodiments, the lipid is an unsaturated fatty acid. In some embodiments, the non-saturated fatty acid is oleic acid, linoleic acid, or palmitoleic acid.

In some embodiments, the disclosure provides for a composition comprising a population of insulin-positive cells and MCDB 131. In some embodiments, the disclosure provides for a method of contacting a population of insulin-positive cells with MCDB 131. In some embodiments, the disclosure provides for a composition comprising a population of insulin-positive cells and DMEM/F12. In some embodiments, the disclosure provides for a method of contacting a population of insulin-positive cells with DMEM/F12. In some embodiments, the disclosure provides for a composition comprising a population of insulin-positive cells and zinc. In some embodiments, the disclosure provides for a method of contacting a population of insulin-positive cells with zinc. In some embodiments, the disclosure provides for a composition comprising a population of insulin-positive cells and ZnSO₄. In some embodiments, the disclosure provides for a method of contacting a population of insulin-positive cells with ZnSO₄.

In some embodiments, the disclosure provides for a composition comprising a population of insulin-positive cells and at least one metabolite. In some embodiments, the disclosure provides for a method of contacting a population of insulin-positive cells with at least one metabolite. In some embodiments, the at least one metabolite is glutamate, acetate, b-hydroxybutarate, L-carnitine, taurine, formate, or biotin. In some embodiments, the disclosure provides for a composition comprising a population of insulin-positive cells and one, two, three, four, five, six, or seven of glutamate, acetate, b-hydroxybutarate, L-carnitine, taurine, formate, or biotin. In some embodiments, the disclosure provides for a method of contacting a population of insulin-positive cells with one, two, three, four, five, six, or seven of glutamate, acetate, b-hydroxybutarate, L-carnitine, taurine, formate, or biotin.

In some embodiments, the disclosure provides for a composition comprising a population of insulin-positive cells and at least one amino acid. In some embodiments, the disclosure provides for a method of contacting a population of insulin-positive cells with at least one amino acid. In some embodiments, the at least one amino acid is alanine, glutamate, glycine, proline, threonine, or tryptophan. In some embodiments, the at least one amino acid is arginine, histidine, lysine, aspartic acid, glutamic acid, serine, asparagine, glutamine, cysteine, selenocysteine, alanine, valine, isoleucine, leucine, methionine, phenylalanine, tyrosine, glutamate, glycine, proline, threonine, or tryptophan. In some embodiments, the disclosure provides for a composition comprising a population of insulin-positive cells and at least one vitamin. In some embodiments, the disclosure provides for a method of contacting a population of insulin-positive cells with at least one vitamin. In some embodiments, the at least one vitamin is biotin or riboflavin.

In some embodiments, the disclosure provides for a composition comprising a population of insulin-positive cells and a monoglyceride lipase (MGLL) inhibitor. In some embodiments, the disclosure provides for a method of contacting a population of insulin-positive cells with at least one vitamin. In some embodiments, the MGLL inhibitor is any of JJKK048, KML29, NF1819, JW642, JZL184, JZL195, JZP361, pristimerin, or URB602, or derivatives thereof.

### Differentiation Factors

Aspects of the disclosure relate to contacting progenitor cells (e.g., stem cells, e.g., iPS cells, definitive endoderm cells, primitive gut tube cells, PDX1-positive, NKX6.1-negative pancreatic progenitor cells, PDX1-positive, NKX6.1-positive pancreatic progenitor cells, insulin-positive endocrine cells) with one or more β cell differentiation factors, for example, to induce the maturation of the insulin-positive endocrine cells or differentiation of other progenitor cells into SC-β cells (e.g., mature pancreatic β cells). In some embodiments, the differentiation factor(s) can induce the differentiation of pluripotent cells (e.g., iPSCs or hESCs) into definitive endoderm cells, e.g., in accordance with a method described herein. In some embodiments, the differentiation factor(s) can induce the differentiation of definitive endoderm cells into primitive gut tube cells, e.g., in accordance with a method described herein. In some embodiments, the differentiation factor(s) can induce the differentiation of primitive gut tube cells into PDX1-positive, NKX6.1-negative pancreatic progenitor cells, e.g., in accordance with a method described herein. In some embodiments, the differentiation factor(s) can induce the differentiation of PDX1-positive, NKX6.1-negative pancreatic progenitor cells into NKX6-1-positive pancreatic progenitor cells, e.g., in accordance with a method described herein. In some embodiments, the differentiation factor(s) can induce the differentiation of NKX6-1-positive pancreatic progenitor cells into insulin-positive endocrine cells, e.g., in accordance with a method described herein. In some embodiments, the differentiation factor(s) can induce the maturation of insulin-positive endocrine cells into SC-β cells, e.g., in accordance with a method described herein.

At least one differentiation factor described herein can be used alone, or in combination with other differentiation actors, to generate SC-β cells according to the methods as disclosed herein. In some embodiments, at least two, at least three, at least four, at least five, at least six, at least seven, at least eight, at least nine, or at least ten differentiation factors described herein are used in the methods of generating SC-β cells.

### Transforming Growth Factor-β (TGF-β) Superfamily

Aspects of the disclosure relate to the use of growth factors from the transforming growth factor-β (TGF-β) superfamily as differentiation factors. The “TGF-β superfamily” means proteins having structural and functional characteristics of known TGFβ family members. The TGFβ family of proteins can include the TGFβ series of proteins, the Inhibins (including Inhibin A and Inhibin B), the Activins (including Activin A, Activin B, and Activin AB), MIS (Müllerian inhibiting substance), BMP (bone morphogenetic proteins), dpp (decapentaplegic), Vg-1, MNSF (monoclonal nonspecific suppressor factor), and others. Activity of this family of proteins can be based on specific binding to certain receptors on various cell types. Members of this family can share regions of sequence identity, particularly at the C-terminus, that correlate to their function. The TGFβ family can include more than one hundred distinct proteins, all sharing at least one region of amino acid sequence identity. Members of the family that can be used in the method disclosed herein can include, but are not limited to, the following proteins, as identified by their GenBank accession numbers: P07995, P18331, P08476, Q04998, P03970, P43032, P55102, P27092, P42917, P09529, P27093, P04088, Q04999, P17491, P55104, Q9WUK5, P55103, O88959, 008717, P58166, 061643, P35621, P09534, P48970, QONR23, P25703, P30884, P12643, P49001, P21274, O46564, 019006, P22004, P20722, Q04906, Q07104, P30886, P18075, P23359, P22003, P34821, P49003, Q90751, P21275, Q06826, P30885, P34820, Q29607, P12644, Q90752, O46576, P27539, P48969, Q26974, P07713, P91706, P91699, P27091, O42222, Q24735, P20863, 018828, P55106, Q9PTQ2, O14793, O08689, O42221, O18830, O18831, O18836, O35312, O42220, P43026, P43027, P43029, O95390, Q9R229, O93449, Q9Z1W4, Q9BDW8, P43028, Q7Z4P5, P50414, P17246, P54831, P04202, P01137, P09533, P18341, 019011, Q9Z1Y6, P07200, Q9Z217, O95393, P55105, P30371, Q9MZE2, Q07258, Q96S42, P97737, AAA97415.1, NP-776788.1, NP-058824.1, EAL24001.1, 1 S4Y, NP-001009856.1, NP-1-032406.1, NP-999193.1, XP-519063.1, AAG17260.1, CAA40806.1, NP-1-001009458.1, AAQ55808.1, AAK40341.1, AAP33019.1, AAK21265.1, AAC59738.1, CAI46003.1, B40905, AAQ55811.1, AAK40342.1, XP-540364.1, P55102, AAQ55810.1, NP-990727.1, CAA51163.1, AAD50448.1, JC4862, PN0504, BAB17600.1, AAH56742.1, BAB17596.1, CAG06183.1, CAG05339.1, BAB17601.1, CAB43091.1, A36192, AAA49162.1, AAT42200.1, NP-789822.1, AAA59451.1, AAA59169.1, XP-541000.1, NP-990537.1, NP-1-002184.1, AAC14187.1, AAP83319.1, AAA59170.1, BAB16973.1, AAM66766.1, WFPGBB, 1201278C, AAH30029.1, CAA49326.1, XP-344131.1, AA-148845.1, XP-1-148966.3, 148235, B41398, AAH77857.1, AAB26863.1, 1706327A, BAA83804.1, NP-571143.1, CAG00858.1, BAB17599.1, BAB17602.1, AAB61468.1, PN0505, PN0506, CAB43092.1, BAB17598.1, BAA22570.1, BAB16972.1, BAC81672.1, BAA12694.1, BAA08494.1, B36192, C36192, BAB16971.1, NP-034695.1, AAA49160.1, CAA62347.1, AAA49161.1, AAD30132.1, CAA58290.1, NP-005529.1, XP-522443.1, AAM27448.1, XP-538247.1, AAD30133. I, AAC36741.1, AAH10404.1, NP-032408.1, AAN03682.1, XP-509161.1, AAC32311.1, NP-651942.2, AAL51005.1, AAC39083.1, AAH85547.1, NP-571023.1, CAF94113.1, EAL29247.1, AAW30007.1, AAH90232.1, A29619, NP-001007905.1, AAH73508.1, AADO2201.1, NP-999793.1, NP-990542.1, AAF19841.1, AAC97488.1, AAC60038.1, NP 989197.1, NP-571434.1, EAL41229.1, AAT07302.1, CAI19472.1, NP-031582.1, AAA40548.1, XP-535880.1, NP-1-037239.1, AAT72007.1, XP-418956.1, CAA41634.1, BAC30864.1, CAA38850.1, CAB81657.2, CAA45018.1, CAA45019.1, BAC28247.1, NP-031581.1, NP-990479.1, NP-999820.1, AAB27335.1, S45355, CAB82007.1, XP-534351.1, NP-058874.1, NP-031579.1, 1REW, AAB96785.1, AAB46367.1, CAA05033.1, BAA89012.1, IES7, AAP20870.1, BAC24087.1, AAG09784.1, BAC06352.1, AAQ89234.1, AAM27000.1, AAH30959.1, CAGO1491.1, NP-571435.1, 1REU, AAC60286.1, BAA24406.1, A36193, AAH55959.1, AAH54647.1, AAH90689.1, CAG09422.1, BAD16743.1, NP-032134.1, XP-532179.1, AAB24876.1, AAH57702.1, AAA82616.1, CAA40222.1, CAB90273.2, XP-342592.1, XP-534896.1, XP-534462.1, 1LXI, XP-417496.1, AAF34179.1, AAL73188.1, CAF96266.1, AAB34226.1, AAB33846.1, AAT12415.1, AA033819.1, AAT72008.1, AAD38402.1, BAB68396.1, CAA45021.1, AAB27337.1, AAP69917.1, AATI2416.1, NP-571396.1, CAA53513.1, AA033820.1, AAA48568.1, BAC02605.1, BAC02604.1, BAC02603.1, BAC02602.1, BAC02601.1, BAC02599.1, BAC02598.1, BAC02597.1, BAC02595.1, BAC02593.1, BAC02592.1, BAC02590.1, AAD28039.1, AAP74560.1, AAB94786.1, NP-001483.2, XP-528195.1, NP-571417.1, NP-001001557. I, AAH43222.1, AAM33143.1, CAG10381.1, BAA31132.1, EAL39680.1, EAA12482.2, P34820, AAP88972.1, AAP74559.1, CAI16418.1, AAD30538.1, XP-345502.1, NP-1-038554.1, CAG04089.1, CAD60936.2, NP-031584.1, B55452, AAC60285.1, BAA06410.1, AAH52846.1, NP-031580.1, NP-1-036959.1, CAA45836.1, CAA45020.1, Q29607, AAB27336.1, XP-547817.1, AAT12414.1, AAM54049.1, AAH78901.1, AA025745.1, NP-570912.1, XP-392194.1, AAD20829.1, AAC97113.1, AAC61694.1, AAH60340.1, AAR97906.1, BAA32227.1, BAB68395.1, BAC02895.1, AAWS 1451.1, AAF82188.1, XP-544189.1, NP-990568.1, BAC80211.1, AAW82620.1, AAF99597.1, NP-571062.1, CAC44179.1, AAB97467.1, AAT99303.1, AAD28038.1, AAH52168.1, NP-001004122.1, CAA72733.1, NP-032133.2, XP-394252.1, XP-224733.2, JH0801, AAP97721.1, NP-989669.1, S43296, P43029, A55452, AAH32495.1, XP-542974.1, NP-032135.1, AAK30842.1, AAK27794.1, BAC30847.1, EAA12064.2, AAP97720.1, XP-525704.1, AAT07301.1, BAD07014.1, CAF94356.1, AAR27581.1, AAG13400.1, AAC60127.1, CAF92055.1, XP-540103.1, AA020895.1, CAF97447.1, AAS01764.1, BAD08319.1, CAA10268.1, NP-998140.1, AAR03824.1, AAS48405.1, AAS48403.1, AAK53545.1, AAK84666.1, XP-395420.1, AAK56941.1, AAC47555.1, AAR88255.1, EAL33036.1, AAW47740.1, AAW29442.1, NP-722813.1, AARO8901.1, AAO 15420.2, CAC59700.1, AAL26886.1, AAK71708.1, AAK71707.1, CAC51427.2, AAK67984.1, AAK67983.1, AAK28706.1, P07713, P91706, P91699, CAG02450.1, AAC47552.1, NP-005802.1, XP-343149.1, AW34055.1, XP-538221.1, AAR27580.1, XP-125935.3, AAF21633.1, AAF21630.1, AAD05267.1, Q9Z1 W4, NP-1-031585.2, NP-571094.1, CAD43439.1, CAF99217.1, CAB63584.1, NP-722840.1, CAE46407.1, XP-1-417667.1, BAC53989.1, BAB19659.1, AAM46922.1, AAA81169.1, AAK28707.1, AAL05943.1, AAB17573.1, CAH25443.1, CAG10269.1, BAD16731.1, EAA00276.2, AAT07320.1, AAT07300.1, AAN15037.1, CAH25442.1, AAK08152.2, 2009388A, AAR12161.1, CAGO1961.1, CAB63656.1, CAD67714.1, CAF94162.1, NP-477340.1, EAL24792.1, NP-1-001009428.1, AAB86686.1, AAT40572.1, AAT40571.1, AAT40569.1, NP-033886.1, AAB49985.1, AAG39266.1, Q26974, AAC77461.1, AAC47262.1, BAC05509.1, NP-055297.1, XP-546146.1, XP-525772.1, NP-060525.2, AAH33585.1, AAH69080.1, CAG12751.1, AAH74757.2, NP-034964.1, NP-038639.1, 042221, AAF02773.1, NP-062024.1, AAR18244.1, AAR14343.1, XP-228285.2, AAT40573.1, AAT94456.1, AAL35278.1, AAL35277.1, AAL17640.1, AAC08035.1, AAB86692.1, CAB40844.1, BAC38637.1, BAB16046.1, AAN63522.1, NP-571041.1, AAB04986.2, AAC26791.1, AAB95254.1, BAA11835.1, AAR18246.1, XP-538528.1, BAA31853.1, AAK18000.1, XP-1-420540.1, AAL35276.1, AAQ98602.1, CAE71944.1, AAW50585.1, AAV63982.1, AAW29941.1, AAN87890.1, AAT40568.1, CAD57730.1, AAB81508.1, AAS00534.1, AAC59736.1, BAB79498.1, AAA97392.1, AAP85526.1, NP-999600.2, NP-878293.1, BAC82629.1, CAC60268.1, CAG04919.1, AAN10123.1, CAA07707.1 AAK20912.1, AAR88254.1, CAC34629.1, AAL35275.1, AAD46997. I, AAN03842.1, NP-571951.2, CAC50881.1, AAL99367.1, AAL49502.1, AAB71839.1, AAB65415.1, NP-624359.1, NP-990153.1, AAF78069.1, AAK49790.1, NP-919367.2, NP-001192.1, XP-544948.1, AAQ18013.1, AAV38739.1, NP-851298.1, CAA67685.1, AAT67171.1, AAT37502.1, AAD27804.1, AAN76665.1, BAC11909.1, XP-1-421648.1, CAB63704.1, NP-037306.1, A55706, AAF02780.1, CAG09623.1, NP-067589.1, NP-035707.1, AAV30547.1, AAP49817.1, BAC77407.1, AAL87199.1, CAG07172.1, B36193, CAA33024.1, NP-1-001009400.1, AAP36538.1, XP-512687.1, XP-510080.1, AAH05513.1, 1KTZ, AAH14690.1, AAA31526.1.

The growth factor from the TGF-β superfamily in the methods and compositions provided herein can be naturally obtained or recombinant. In some embodiments, the growth factor from the TGF-β superfamily comprises Activin A. The term “Activin A” can include fragments and derivatives of Activin A. Non-limiting exemplary sequences of Activin A are listed in Table 2. In some embodiments, the growth factor from the TGF-β superfamily can comprise a polypeptide having an amino acid sequence at least 30%, at least 40%, at least 50%, at least 60%, at least 70%, at least 80%, at least 90%, at least 95%, or at least 99%, or greater identical to the sequence of SEQ ID NO: 1. In some embodiments, the growth factor from the TGF-β superfamily can comprise a polypeptide having the amino acid sequence of SEQ ID NO: 1. In some embodiments, the growth factor from the TGF-β superfamily can comprise a polypeptide having an amino acid sequence at least 30%, at least 40%, at least 50%, at least 60%, at least 70%, at least 80%, at least 90%, at least 95%, or at least 99%, or greater identical to the sequence of any one of SEQ ID NOs: 2-15.

In some embodiments, the growth factor from the TGF-β superfamily comprises growth differentiation factor 8 (GDF8). The term “GDF8” can include fragments and derivatives of GDF8. The sequences of GDF8 polypeptides are available to the skilled artisan. In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF8 polypeptide sequence (GenBank Accession EAX10880).

In some embodiments, the growth factor from the TGF-β superfamily comprises a growth factor that is closely related to GDF8, e.g., growth differentiation factor 11 (GDF11). In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF11 polypeptide sequence (GenBank Accession AAF21630).

In some embodiments, the growth factor from the TGF-β superfamily can be replaced with an agent mimics the at least one growth factor from the TGF-β superfamily. Exemplary agents that mimic the at least one growth factor from the TGF-β superfamily, include, without limitation, IDE1 and IDE2.

### Bone Morphogenetic Protein (BMP) Signaling Pathway Inhibitors

Aspects of the disclosure relate to the use of BMP signaling pathway inhibitors as β cell differentiation factors. The BMP signaling family is a diverse subset of the TGF-β superfamily (Sebald et al. Biol. Chem. 385:697-710, 2004). Over twenty known BMP ligands are recognized by three distinct type II (BMPRII, ActRIIa, and ActRIIb) and at least three type I (ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner (Nohe et al. Cell Signal 16:291-299, 2004). Soluble BMP antagonists such as noggin, chordin, gremlin, and follistatin limit BMP signaling by ligand sequestration.

In some embodiments, the BMP signaling pathway inhibitor in the methods and composition provided herein comprises DMH-1, or a derivative, analogue, or variant thereof. In some embodiments, the BMP signaling pathway inhibitor in the methods and composition provided herein comprises the following compound or a derivative, analogue, or variant of the following compound:

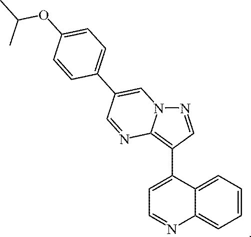

In some embodiments, the BMP signaling pathway inhibitor in the methods and composition provided herein comprises LDN193189 (also known as LDN193189, 1062368-24-4, LDN-193189, DM 3189, DM-3189, IUPAC Name: 4-[6-(4-piperazin-1-ylphenyl)pyrazolo[1,5-a]pyrimidin-3-yl]quinolone). In some embodiments, the BMP signaling pathway inhibitor in the methods and composition provided herein comprises the following compound or a derivative, analogue, or variant of the following compound:

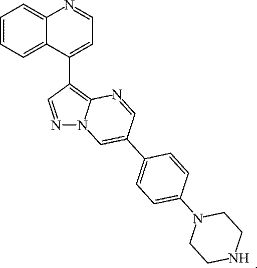

In some cases, DMH-1 can be more selective as compared to LDN193189. In some embodiments of the present disclosure, DMH-1 can be particularly useful for the methods provided herein. In some embodiments, the methods and compositions provided herein exclude use of LDN193189. In some embodiments, the methods and compositions provided herein exclude use of LDN193189, or a derivative, analogue, or variant thereof for generating PDX1-positive, NKX6.1-negative pancreatic progenitor cells from primitive gut tube cells. In some embodiments, the methods and compositions provided herein relate to use of DMH-1, or a derivative, analogue, or variant thereof for generating PDX1-positive, NKX6.1-negative pancreatic progenitor cells from primitive gut tube cells.

In some embodiments, the BMP signaling pathway inhibitor in the methods and composition provided herein comprise an analog or derivative of LDN193189, e.g., a salt, hydrate, solvent, ester, or prodrug of LDN193189. In some embodiments, a derivative (e.g., salt) of LDN193189 comprises LDN193189 hydrochloride.

In some embodiments, the BMP signaling pathway inhibitor in the methods and composition provided herein comprises a compound of Formula I from U.S. Pat. Publication No. 2011/0053930.

### TGF-β Signaling Pathway Inhibitors

Aspects of the disclosure relate to the use of TGF-β signaling pathway inhibitors as β cell differentiation factors.

In some embodiments, the TGF-β signaling pathway comprises TGF-β receptor type I kinase (TGF-β RI) signaling. In some embodiments, the TGF-β signaling pathway inhibitor comprises ALK5 inhibitor II (CAS 446859-33-2, an ATP-competitive inhibitor of TGF-B RI kinase, also known as “ALK5i”, RepSox, IUPAC Name: 2-[5-(6-methylpyridin-2-yl)-1H-pyrazol-4-yl]-1,5-naphthyridine. In some embodiments, the TGF-β signaling pathway inhibitor is an analog or derivative of ALK5 inhibitor II.

In some embodiments, the analog or derivative of ALK5 inhibitor II is a compound of Formula I as described in U.S. Pat Publication No. 2012/0021519, incorporated by reference herein in its entirety.

In some embodiments, the TGF-β signaling pathway inhibitor in the methods and compositions provided herein is a TGF-β receptor inhibitor described in U.S. Pat. Publication No. 2010/0267731. In some embodiments, the TGF-β signaling pathway inhibitor in the methods and compositions provided herein comprises an ALK5 inhibitor described in U.S. Pat. Publication Nos. 2009/0186076 and 2007/0142376. In some embodiments, the TGF-β signaling pathway inhibitor in the methods and compositions provided herein is A 83-01. In some embodiments, the TGF-β signaling pathway inhibitor in the methods and compositions provided herein is not A 83-01. In some embodiments, the compositions and methods described herein exclude A 83-01. In some embodiments, the TGF-β signaling pathway inhibitor in the methods and compositions provided herein is SB 431542. In some embodiments, the TGF-β signaling pathway inhibitor is not SB 431542. In some embodiments, the compositions and methods described herein exclude SB 431542. In some embodiments, the TGF-β signaling pathway inhibitor in the methods and compositions provided herein is D 4476. In some embodiments, the TGF-βsignaling pathway inhibitor is not D 4476. In some embodiments, the compositions and methods described herein exclude D 4476. In some embodiments, the TGF-β signaling pathway inhibitor in the methods and compositions provided herein is GW 788388. In some embodiments, the TGF-β signaling pathway inhibitor is not GW 788388. In some embodiments, the compositions and methods described herein exclude GW 788388. In some embodiments, the TGF-β signaling pathway inhibitor in the methods and compositions provided herein is LY 364947. In some embodiments, the TGF-β signaling pathway inhibitor is not LY 364947. In some embodiments, the compositions and methods described herein exclude LY 364947. In some embodiments, the TGF-β signaling pathway inhibitor in the methods and compositions provided herein is LY 580276. In some embodiments, the TGF-β signaling pathway inhibitor is not LY 580276. In some embodiments, the compositions and methods described herein exclude LY 580276. In some embodiments, the TGF-β signaling pathway inhibitor in the methods and compositions provided herein is SB 525334. In some embodiments, the TGF-β signaling pathway inhibitor is not SB 525334. In some embodiments, the compositions and methods described herein exclude SB 525334. In some embodiments, the TGF-β signaling pathway inhibitor in the methods and compositions provided herein is SB 505124. In some embodiments, the TGF-β signaling pathway inhibitor is not SB 505124. In some embodiments, the compositions and methods described herein exclude SB 505124. In some embodiments, the TGF-β signaling pathway inhibitor in the methods and compositions provided herein is SD 208. In some embodiments, the TGF-β signaling pathway inhibitor is not SD 208. In some embodiments, the compositions and methods described herein exclude SD 208. In some embodiments, the TGF-β signaling pathway inhibitor in the methods and compositions provided herein is GW 6604. In some embodiments, the TGF-β signaling pathway inhibitor is not GW 6604. In some embodiments, the compositions and methods described herein exclude GW 6604. In some embodiments, the TGF-β signaling pathway inhibitor in the methods and compositions provided herein is GW 788388. In some embodiments, the TGF-β signaling pathway inhibitor in the methods and compositions provided herein is not GW 788388. In some embodiments, the compositions and methods described herein exclude GW 788388.

From the collection of compounds described above, the following can be obtained from various sources: LY-364947, SB-525334, SD-208, and SB-505124 available from Sigma, P.O. Box 14508, St. Louis, Mo., 63178-9916; 616452 and 616453 available from Calbiochem (EMD Chemicals, Inc.), 480 S. Democrat Road, Gibbstown, N.J., 08027; GW788388 and GW6604 available from GlaxoSmithKline, 980 Great West Road, Brentford, Middlesex, TW8 9GS, United Kingdom; LY580276 available from Lilly Research, Indianapolis, Ind. 46285; and SM16 available from Biogen Idec, P.O. Box 14627, 5000 Davis Drive, Research Triangle Park, N.C., 27709-4627.

### WNT Signaling Pathway

Aspects of the disclosure relate to the use of activators of the WNT signaling pathway as β cell differentiation factors.

In some embodiments, the WNT signaling pathway activator in the methods and compositions provided herein comprises CHIR99021. In some embodiments, the WNT signaling pathway activator in the methods and compositions provided herein comprises a derivative of CHIR99021, e.g., a salt of CHIR99021, e.g., trihydrochloride, a hydrochloride salt of CHIR99021. In some embodiments, the WNT signaling pathway activator in the methods and compositions provided herein comprises Wnt3a recombinant protein. In some embodiments, the WNT signaling pathway activator in the methods and compositions provided herein comprises a glycogen synthase kinase 3 (GSK3) inhibitor. Exemplary GSK3 inhibitors include, without limitation, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, TWS 119, and analogs or derivatives of any of these. In certain embodiments, the methods, compositions, and kits disclosed herein exclude a WNT signaling pathway activator.

### Fibroblast Growth Factor (FGF) Family

Aspects of the disclosure relate to the use of growth factors from the FGF family as β cell differentiation factors.

In some embodiments, the growth factor from the FGF family in the methods and compositions provided herein comprises keratinocyte growth factor (KGF). The polypeptide sequences of KGF are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human KGF polypeptide sequence (GenBank Accession AAB21431). In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the sequence of SEQ ID NO: 16.

In some embodiments, the growth factor from the FGF family in the methods and composition provided herein comprises FGF2. The polypeptide sequences of FGF2 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF2 polypeptide sequence (GenBank Accession NP__001997).

In some embodiments, the at least one growth factor from the FGF family in the methods and composition provided herein comprises FGF8B. The polypeptide sequences of FGF8B are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF8B polypeptide sequence (GenBank Accession AAB40954).

In some embodiments, the at least one growth factor from the FGF family in the methods and composition provided herein comprises FGF10. The polypeptide sequences of FGF10 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF10 polypeptide sequence (GenBank Accession CAG46489).

In some embodiments, the at least one growth factor from the FGF family in the methods and composition provided herein comprises FGF21. The polypeptide sequences of FGF21 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF21 polypeptide sequence (GenBank Accession AAQ89444.1).

### Sonic Hedgehog (SHH) Signaling Pathway

Aspects of the disclosure relate to the use of SHH signaling pathway inhibitors as β cell differentiation factors.

In some embodiments, the SHH signaling pathway inhibitor in the methods and composition provided herein comprises Sant1. In some embodiments, the SHH signaling pathway inhibitor in the methods and composition provided herein comprises SANT2. In some embodiments, the SHH signaling pathway inhibitor in the methods and composition provided herein comprises SANT3. In some embodiments, the SHH signaling pathway inhibitor in the methods and composition provided herein comprises SANT4. In some embodiments, the SHH signaling pathway inhibitor comprises Cur61414. In some embodiments, the SHH signaling pathway inhibitor in the methods and composition provided herein comprises forskolin. In some embodiments, the SHH signaling pathway inhibitor in the methods and composition provided herein comprises tomatidine. In some embodiments, the SHH signaling pathway inhibitor in the methods and composition provided herein comprises AY9944. In some embodiments, the SHH signaling pathway inhibitor in the methods and composition provided herein comprises triparanol. In some embodiments, the SHH signaling pathway inhibitor in the methods and composition provided herein comprises compound A or compound B (as disclosed in U.S. Pub. No. 2004/0060568). In some embodiments, the SHH signaling pathway inhibitor in the methods and composition provided herein comprises a steroidal alkaloid that antagonizes hedgehog signaling (e.g., cyclopamine or a derivative thereof) as disclosed in U.S. Pub. No. 2006/0276391. In certain embodiments, the methods, compositions, and kits disclosed herein exclude a SHH signaling pathway inhibitor.

### Rho Kinase (ROCK) Signaling Pathway

Aspects of the disclosure relate to the use of ROCK signaling pathway inhibitors (ROCK inhibitors) as β cell differentiation factors.

In some embodiments, the ROCK inhibitor in the methods and composition provided herein comprises Y-27632 or Thiazovivin. In some embodiments, the ROCK inhibitor in the methods and composition provided herein comprises Thiazovivin. In some embodiments, the ROCK inhibitor in the methods and composition provided herein comprises Y-27632. In some cases, the ROCK inhibitor in the methods and composition provided herein comprises the following compound or a derivative thereof:

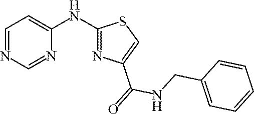 [SMILES: O=C(NCc1ccccc1)c1csc(Nc2ccncn2)n1]

In some cases, the ROCK inhibitor in the methods and composition provided herein comprises the following compound or a derivative thereof:

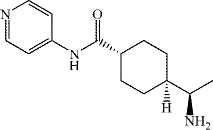 [SMILES: C[C@@H](N)[C@H]1CC[C@H](C(=O)Nc2ccncc2)CC1]

Non-limiting examples of ROCK inhibitor that can be used in the methods and compositions provided herein include Thiazovivin, Y-27632, Fasudil/HA1077, H-1152, Ripasudil, Y39983, Wf-536, SLx-2119, Azabenzimidazole-aminofurazans, DE-104, Olefins, Isoquinolines, Indazoles, and pyridinealkene derivatives, ROKα inhibitor, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, and quinazoline.

### Retinoic Acid Signaling Pathway

Aspects of the disclosure relate to the use of modulators of retinoic acid signaling as β cell differentiation factors.

In some embodiments, the modulator of retinoic acid signaling in the methods and composition provided herein comprises an activator of retinoic acid signaling. In some embodiments, the RA signaling pathway activator in the methods and composition provided herein comprises retinoic acid. In some embodiments, the RA signaling pathway activator in the methods and composition provided herein comprises a retinoic acid receptor agonist. Exemplary retinoic acid receptor agonists in the methods and composition provided herein include, without limitation, CD 1530, AM 580, TTNPB, CD 437, Ch 55, BMS 961, AC 261066, AC 55649, AM 80, BMS 753, tazarotene, adapalene, and CD 2314.

In some embodiments, the modulator of retinoic acid signaling in the methods and composition provided herein comprises an inhibitor of retinoic acid signaling. In some embodiments, the retinoic acid signaling pathway inhibitor comprises DEAB (IUPAC Name: 2-[2-(diethylamino)ethoxy]-3-prop-2-enylbenzaldehyde). In some embodiments, the retinoic acid signaling pathway inhibitor comprises an analog or derivative of DEAB.

In some embodiments, the retinoic acid signaling pathway inhibitor in the methods and composition provided herein comprises a retinoic acid receptor antagonist. In some embodiments, the retinoic acid receptor antagonist in the methods and composition provided herein comprises (E)-4-[2-(5,6-dihydro-5,5-dimethyl-8-phenyl-2-naphthalenyl)ethenyl]benzoic acid, (E)-4-[[(5,6-dihydro-5,5-dimethyl-8-phenylethynyl)-2-naphthalenyl]ethenyl]benzoic acid, (E)-4-[2-[5,6-dihydro-5,5-dimethyl-8-(2-naphthalenyl)-2-naphthalenyl]ethenyl]-benzoic acid, and (E)-4-[2-[5,6-dihydro-5,5-dimethyl-8-(4-methoxyphenyl)-2-naphthalenyl]ethenyl]benzoic acid. In some embodiments, the retinoic acid receptor antagonist comprises BMS 195614 (CAS#253310-42-8), ER 50891 (CAS#187400-85-7), BMS 493 (CAS#170355-78-9), CD 2665 (CAS#170355-78-9), LE 135 (CAS#155877-83-1), BMS 453 (CAS #166977-43-1), or MM 11253 (CAS#345952-44-5).

In certain embodiments, the methods, compositions, and kits disclosed herein exclude a modulator of retinoic acid signaling. In certain embodiments, the methods, compositions, and kits disclosed herein exclude a retinoic acid signaling pathway activator. In certain embodiments, the methods, compositions, and kits disclosed herein exclude a retinoic acid signaling pathway inhibitor.

### Protein Kinase C Activators

Aspects of the disclosure relate to the use of protein kinase C activators as β cell differentiation factors. Protein kinase C is one of the largest families of protein kinase enzymes and is composed of a variety of isoforms. Conventional isoforms include a, βI, βII, γ; novel isoforms include δ, ε, η, Θ; and atypical isoforms include ξ, and √λ. PKC enzymes are primarily cytosolic but translocate to the membrane when activated. In the cytoplasm, PKC is phosphorylated by other kinases or autophosphorylated. In order to be activated, some PKC isoforms (e.g., PKC-ε) require a molecule to bind to the diacylglycerol (“DAG”) binding site or the phosphatidylserine (“PS”) binding site. Others are able to be activated without any secondary binding messengers at all. PKC activators that bind to the DAG site include, but are not limited to, bryostatin, picologues, phorbol esters, aplysiatoxin, and gnidimacrin. PKC activators that bind to the PS site include, but are not limited to, polyunsaturated fatty acids and their derivatives. It is contemplated that any protein kinase C activator that is capable, either alone or in combination with one or more other β cell differentiation factors, of inducing the differentiation of at least one insulin-producing, endocrine cell or precursor thereof into a SC-β cell can be used in the methods, compositions, and kits described herein.

In some embodiments, any of the PKC activators disclosed herein is a PKC activator capable of binding to a DAG binding site on a PKC. In some embodiments, the PKC activator is capable of binding to a C 1 domain of a PKC. In some embodiments, the PKC activator is a benzolactam-derivative. In some embodiments, the benzolactam-derivative is ((2S,5S)-(E,E)-8-(5-(4-(Trifluoromethyl)phenyl)-2,4-pentadienoylamino)benzolactam), which may be referred to herein as TPPB or TPB. In some embodiments, contacting a population of cells with a benzolactam-derivative PKC activator (e.g., TPPB) increases cell yield as compared to a population of cells not treated with the benzolactam-derivative PKC activator. In some embodiments, the PKC activator is a phorbol ester. In some embodiments, the phorbol ester is Phorbol 12,13-dibutyrate, which may be referred to herein as PDBU or PdbU. In some embodiments, contacting a population of cells with a benzolactam-derivative PKC activator (e.g., TPPB) increases cell yield as compared to a population of cells treated with a phorbol ester PKC activator (e.g., PdbU). In some embodiments, the PKC activator in the methods and composition provided herein comprises PdbU. In some embodiments, the PKC activator in the methods and composition provided herein comprises TPB. In some embodiments, the PKC activator in the methods and composition provided herein comprises cyclopropanated polyunsaturated fatty acids, cyclopropanated monounsaturated fatty acids, cyclopropanated polyunsaturated fatty alcohols, cyclopropanated monounsaturated fatty alcohols, cyclopropanated polyunsaturated fatty acid esters, cyclopropanated monounsaturated fatty acid esters, cyclopropanated polyunsaturated fatty acid sulfates, cyclopropanated monounsaturated fatty acid sulfates, cyclopropanated polyunsaturated fatty acid phosphates, cyclopropanated monounsaturated fatty acid phosphates, macrocyclic lactones, DAG derivatives, isoprenoids, octylindolactam V, gnidimacrin, iripallidal, ingenol, napthalenesulfonamides, diacylglycerol kinase inhibitors, fibroblast growth factor 18 (FGF-18), insulin growth factor, hormones, and growth factor activators, as described in WIPO Pub. No. WO/2013/071282. In some embodiments, the bryostain comprises bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In certain embodiments, the methods, compositions, and kits disclosed herein exclude a protein kinase C activator.

### γ-Secretase Inhibitors

Aspects of the disclosure relate to the use of γ-secretase inhibitors as β cell differentiation factors.

In some embodiments, the γ-secretase inhibitor in the methods and composition provided herein comprises XXI. In some embodiments, the γ-secretase inhibitor in the methods and composition provided herein comprises DAPT. Additional exemplary γ-secretase inhibitors in the methods and composition provided herein include, without limitation, the γ-secretase inhibitors described in U.S. Pat. Nos. 7,049,296, 8,481,499, 8,501,813, and WIPO Pub. No. WO/2013/052700. In certain embodiments, the methods (or specific steps thereof), compositions, and kits disclosed herein exclude a γ-secretase inhibitor.

### Thyroid Hormone Signaling Pathway Activators

Aspects of the disclosure relate to the use of thyroid hormone signaling pathway activators as β cell differentiation factors.

In some embodiments, the thyroid hormone signaling pathway activator in the methods and composition provided herein comprises triiodothyronine (T3). In some embodiments, the thyroid hormone signaling pathway activator in the methods and composition provided herein comprises GC-1. In some embodiments, the thyroid hormone signaling pathway activator in the methods and composition provided herein comprises an analog or derivative of T3 or GC-1. Exemplary analogs of T3 in the methods and composition provided herein include, but are not limited to, selective and non-selective thyromimetics, TRβ selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3’,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine [T4] and triiodothyronine [T3] alanine chain), 3,3’,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (T0AM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA).

In some embodiments, the thyroid hormone signaling pathway activator in the methods and composition provided herein comprises a prodrug or prohormone of T3, such as T4 thyroid hormone (e.g., thyroxine or L-3,5,3’,5′-tetraiodothyronine).

In some embodiments, the thyroid hormone signaling pathway activator in the methods and composition provided herein is an iodothyronine composition described in U.S. Pat. No. 7,163,918.

### Epidermal Growth Factor (EGF) Family

Aspects of the disclosure relate to the use of growth factors from the EGF family as β cell differentiation factors.

In some embodiments, the at least one growth factor from the EGF family in the methods and compositions provided herein comprises betacellulin. In some embodiments, at least one growth factor from the EGF family in the methods and composition provided herein comprises EGF. Epidermal growth factor (EGF) is a 53 amino acid cytokine which is proteolytically cleaved from a large integral membrane protein precursor. In some embodiments, the growth factor from the EGF family in the methods and composition provided herein comprises a variant EGF polypeptide, for example an isolated epidermal growth factor polypeptide having at least 90% amino acid identity to the human wild-type EGF polypeptide sequence, as disclosed in U.S. Pat. No. 7,084,246. In some embodiments, the growth factor from the EGF family in the methods and composition provided herein comprises an engineered EGF mutant that binds to and agonizes the EGF receptor, as is disclosed in U.S. Pat. No. 8,247,531. In some embodiments, the at least one growth factor from the EGF family in the methods and composition provided herein is replaced with an agent that activates a signaling pathway in the EGF family. In some embodiments, the growth factor from the EGF family in the methods and composition provided herein comprises a compound that mimics EGF. In certain embodiments, the methods, compositions, and kits disclosed herein exclude a growth factor from the EGF family. In some embodiments, the growth factor from the EGF family used in the compositions and methods described herein comprises an amino acid sequence that is at least 30%, at least 40%, at least 50%, at least 60%, at least 70%, at least 75%, at least 80%, at least 85%, at least 90%, at least 95%, at least 98% or at least 99% identical to Human betacellulin amino acid sequence (GenBank: AAB25452.1). In some embodiments, the growth factor from the EGF family used in the compositions and methods described herein comprises an amino acid sequence that is at least 30%, at least 40%, at least 50%, at least 60%, at least 70%, at least 75%, at least 80%, at least 85%, at least 90%, at least 95%, at least 98% or at least 99% identical to the amino acid sequence of SEQ ID NO: 17, or a functional fragment thereof. In some embodiments, the growth factor from the EGF family used in the compositions and methods described herein comprises the amino acid sequence of SEQ ID NO: 17.

TABLE 2

Exemplary amino acid sequences of differentiation factors

| SEQ ID NO | Amino acid sequence |
|---|---|
| 1 | GLECDGKVNICCKKQFFVSFKDIGWNDWIIAPSGYHANYCEGECPSHIAG TSGSSLSFHSTVINHYRMRGHSPFANLKSCCVPTKLRPMSMLYYDDGQNI IKKDIQNMIVEECGCS |
| 2 | MPLLWLRGFLLASCWIIVRSSPTPGSEGHSAAPDCPSCALAALPKDVPNS QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEVWLFLKVPKANRTRTKVTIRLFQQQKHPQGSLDTGEEAEEVGLKGE RSELLLSEKVVDARKSTWHVFPVSSSIQRLLDQGKSSLDVRIACEQCQES GASLVLLGKKKKKEEEGEGKKKGGGEGGAGADEEKEQSHRPFLMLQARQS EDHPHRRRRRGLECDGKVNICCKKQFFVSFKDIGWNDWIIAPSGYHANYC EGECPSHIAGTSGSSLSFHSTVINHYRMRGHSPFANLKSCCVPTKLRPMS MLYYDDGQNIIKKDIQNMIVEECGCS |
| 3 | ARQSEDHPHRRRRRGLECDGKVNICCKKQFFVSFKDIGWNDWIIAPSGYH ANYCEGECPSHIAGTSGSSLSFHSTVINHYRMRGHSPFANLKSCCVPTKL RPMSMLYYDDGQNIIKKDIQNMIVEECGCS |
| 4 | GLECDGKVNICCKKQFFVSFKDIGWNDWIIAPSGYHANYCEGECPSHIAG TSGSSLSFHSTVINHYACGHSPFANLKSCCVPTKLRPMSMLYYDDGQNII KKDIQNMIVEECGCS |
| 5 | MPLLWLRGFLLASCWIIVRSSPTPGSEGHSAAPDCPSCALAALPKDVPNS QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEVWLFLKVPKANRTRTKVTIRLFQQQKHPQGSLDTGEEAEEVGLKGE RSELLLSEKVVDARKSTWHVFPVSSSIQRLLDQGKSSLDVRIACEQCQES GASLVLLGKKKKKEEEGEGKKKGGGEGGAGADEEKEQSHRPFLMLQARQS EDHPHRRRRRGLECDGKVNICCKKQFFVSFKDIGWNDWIIAPSGYHANYC EGECPSHIAGTSGSSLSFHSTVINHYRMRGHSPFANLKSCCVPTKLRPMS MLYYDDGQNIIKKDIQNMIVEECGCS |
| 6 | MPLLWLRGFLLASCWIIVRSSPTPGSEGHGSAPDCPSCALATLPKDGPNS QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEVWLFLKVPKANRTRTKVTIRLFQQQKHPQGSLDTGDEAEEMGLKGE RSELLLSEKVVDARKSTWHIFPVSSSIQRLLDQGKSSLDVRIACEQCQES GASLVLLGKKKKKEVDGDGKKKDGSDGGLEEEKEQSHRPFLMLQARQSED HPHRRRRRGLECDGKVNICCKKQFFVSFKDIGWNDWIIAPSGYHANYCEG ECPSHIAGTSGSSLSFHSTVINHYRMRGHSPFANLKSCCVPTKLRPMSML YYDDGQNIIKKDIQNMIVEECGCS |
| 7 | MPLLWLRGFLLASCWIIVRSSPTPGSEGHGAAPDCPSCALATLPKDGPNS QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEVWLFLKVPKANRTRTKVTIRLFQQQKHPQGSLDMGDEAEEMGLKGE RSELLLSEKVVDARKSTWHIFPVSSSIQRLLDQGKSSLDVRIACEQCQES GASLVLLGKKKKKEVDGDGKKKDGSDGGLEEEKEQSHRPFLMLQARQSED HPHRRRRRGLECDGKVNICCKKQFFVSFKDIGWNDWIIAPSGYHANYCEG ECPSHIAGTSGSSLSFHSTVINHYRMRGHSPFANLKSCCVPTKLRPMSML YYDDGQNIIKKDIQNMIVEECGCS |
| 8 | MPLLWKRGFLLVICWIIVRSSPTPGSEGHSSVADCPSCALTTLSKDVPSS QPEMVEAVKKHILNMLHLRDRPNITQPVPKAALLNATKKLHVGKVGDDGY VEIEDDVGRRAEMNEVVEQTSEIITFAESGTPKKTLHFEISKEGSELSVV EHAEVWLFLKVSKANRSRTKVTIRLFQQQRQPKGNSEAAEDMEDMGLKGE RSETLISEKAVDARKSTWHIFPISSSVQRLLDQGQSSLDVRIACDLCQET GASLVLLGKKKKKEDDGEGKEKDGGELTGEEEKEQSHRPFLMMLARHSED RQHRRRERGLECDGKVNICCKKQFFVSFKDIGWSDWIIAPTGYHANYCEE ECPSHIAGTSGSSLSFHSTVINHYRMRGHSPFANLKSCCVPTKLRPMSML YYDDGQNIIKKDIQNMIVEECGCS |
| 9 | MPLLWLRGFLLASCWIIVRSSPTPGSEGHSAAPDCPSCALATLPKDVPNS QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEIWLFLKVPKANRTRSKVTIRLFQQQKHLQGSLDAGEEAEEVGLKGE KSEMLISEKVVDARKSTWHIFPVSSCIQRLLDQGKSSLDIRIACEQCQET GASLVLLGKKKKKEEEGEGKKRDGEGGAGGDEEKEQSHRPFLMLQARQSE DHPHRRRRRGLECDGKVNICCKKQFFVSFKDIGWNDWIIAPSGYHANYCE GECPSHIAGTSGSSLSFHSTVINHYRMRGHSPFANLKSCCVPTKLRPMSM LYYDDGQNIIKKDIQNMIVEECGCS |
| 10 | MPLLWLRGFLLASCWIIVKSSPTPGSEGHSAAPNCPSCALATLPKDVPNA QPEMVEAVKKHILNMLHLKKRPDVTQPVPKAALLNAIRKLHVGKVGENGY VEIEDDIGRRAEMNELMEQTSEIITFAESGTARKTLHFEISKEGSDLSVV ERAEVWLFLKVPKANRTRSKVTIRLLQQQKHPQGSSDTREEAEEADLMEE RSEQLISEKVVDARKSTWHIFPVSSSIQRLLDQGKSSLDIRIACDQCHET GASLVLLGKKKKKEEEGEGKKKDGGEAGAGVDEEKEQSHRPFLMLQARQS EDHPHRRRRRGLECDGKVNICCKKQFFVSFKDIGWNDWIIAPSGYHANYC EGECPSHIAGTSGSSLSFHSTVINQYRLRGHNPFANLKSCCVPTKLRPMS MLYYDDGQNIIKKDIQNMIVEECGCS |
| 11 | MPLLWLRGFLLASCWIIVRSSPTPGSGGHSAAPDCPSCALATLPKDVPNS QPEMVEAVKKHILNMLHLKKRPDVTQPVPKAALLNAIRKLHVGKVGENGY VELEDDIGRRAEMNELMEQTSEIITFAEAGTARKTLRFEISKEGSDLSVV ERAEIWLFLKVPKANRTRTKVSIRLFQQQRRPQGSADAGEEAEDVGFPEE KSEVLISEKVVDARKSTWHIFPVSSSIQRLLDQGKSALDIRTACEQCHET GASLVLLGKKKKKEEEAEGRKRDGEGAGVDEEKEQSHRPFLMLQARQSEE HPHRRRRRGLECDGKVNICCKKQFFVSFKDIGWNDWIIAPSGYHANYCEG ECPSHIAGTSGSSLSFHSTVINHYRMRGHSPFANLKSCCVPTKLRPMSML YYDDGQNIIKKDIQNMIVEECGCS |
| 12 | MPLLWLRGFLLASCWIIVRSSPTPGSEGHSAAPDCPSCALATLPKDVPNS QPEMVEAVKKHILNMLHLKKRPDVTQPVPKAALLNAIRKLHVGKVGENGY VEIEDDIGRRAEMNELMEQTSEIITFAESGTARKTLHFEISQEGSDLSVV ERAEIWLFLKVPKANRTRSKVTIRLFQQQKHLQGSLDAGEEAEEVGLKGE KSEMLISEKVVDARKSTWHIFPVSSCIQRLLDQGKSSLDIRIACEQCQET GASLVLLGKKKRKEEEGEGKKRDGEGGAGGDEEKEQSHRPFLMLQARQSE |
| | DHPHRRRRRGLECDGKVNICCKKQFYVSFKDIGWNDWIIAPSGYHANYCE GECPSHIAGTSGSSLSFHSTVINHYRMRGHSPFANLKSCCVPTKLRPMSM LYYDDGQNIIKKDIQNMIVEECGCS |
| 13 | MPLLWLRGFLLASCWIIVRSSPTPGSEGPGAAPDCPSCALATLPKDVPNS QPEMVEAVKKHILNMLHLKKRPEVTQPVPKAALLNAIRKLHVGKVGENGY VEIEDDIGRRAEMNELMEQTSEIITFAESGTARKTLHFEISKEGSDLSVV ERAEVWLFLKVPKANRTRTKVTIQLLQKQPQGGVDAGEEAEEMGLMEERN EVLISEKVVDARKSTWHIFPVSSSIQRLLDQGKSSLDVRIACEQCHETGA SLVLLGKKKKKEEEGEGKKKDGGDGGAGADEDKEQSHRPFLMLQARQSED HPHRRRRRGLECDGKVNICCKKQFFVSFKDIGWNDWIIAPSGYHANYCEG ECPSHIAGTSGSSLSFHSTVINHYRMRGHSPFANLKSCCVPTKLRPMSML YYDDGQNIIKKDIQNMIVEECGCS |
| 14 | MSPLPLLSGILLLLIRSCSLSAMVTKGSLPMSEQQAGATVCPSCALARFR KGVSESEDEGAQQDVVEAVKRHILNMLHLQERPNITHPVPRAALLNAIRK VHVGRVAKDGSVLIEDEASNRAETEQAEQTEIITFAETGEAPGIVNFLIS KEGGEMSVVDQANVWIFLRLPKGNRTRANVNIRLLLQQGAGEKILAEKSV DTRRSGWHTFPASESVQSLLQRGGSTLSLRVSCPLCADARATPVLVSPGG SEREQSHRPFLMAVVRQMDELSLRRRRKRGLECDGKARVCCKRQFYVNFK DIGWNDWIIAPSGYHANYCEGDCASNVASITGNSLSFHSTVISHYRIRGY SPFTNIKSCCVPTRLRAMSMLYYNEEQKIVKKDIQNMIVEECGCS |
| 15 | MSSLTLVNRGTAALRLFVRGLLTHSSREWLSGDGEPDDPVTPCPSCALAQ RQKDSEEQTDMVEAVKRHILNMLHLNTRPNVTHPVPRAALLNAIRRLHVG RVGEDGTVEMEEDGGGLGEHREQSEEQPFEIITFAEPGDAPDIMKFDISM EGNTLSVVEQANVWLLLKVAKGSRGKGKVSVQLLQHGKADPGSADGPQEA VVSEKTVDTRRSGWHTLPVSRTVQTLLDGDSSMLSLRVSCPMCAEAGAVP ILVPTESNKGKEREQSHRPFLMVVLKPAEEHPHRRSKRGLECDGKIRVCC KRQFYVNFKDIGWSDWIIAPSGYHANYCEGDCPSHVASITGSALSFHSTV INHYRMRGYSPFNNIKSCCVPTRLRAMSMLYYNEEQKIIKKDIQNMIVEE CGCS |
| 16 | MHKWILTWILPTLLYRSCFHIICLVGTISLACNDMTPEQMATNVNCSSPE RHTRSYDYMEGGDIRVRRLFCRTQWYLRIDKRGKVKGTQEMKNNYNIMEI RTVAVGIVAIKGVESEFYLAMNKEGKLYAKKECNEDCNFKELILENHYNT YASAKWTHNGGEMFVALNQKGIPVRGKKTKKEQKTAHFLPMAIT |
| 17 | MDRAARCSGASSLPLLLALALGLVILHCVVADGNSTRSPETNGLLCGDPE ENCAATTTQSKRKGHFSRCPKQYKHYCIKGRCRFVVAEQTPSCVCDEGYI GARCERVDLFYLRGDRGQILVICLIAVMVVFIILVIGVCTCCHPLRKRRK RKKKEEEMETLGKDITPINEDIEETN |

### Epigenetic Modifying Compounds

Aspects of the disclosure relate to the use of epigenetic modifying compound as β cell differentiation factors.

The term “epigenetic modifying compound” can refer to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, can alter DNA accessibility and chromatin structure, thereby regulating patterns of gene expression. These processes can be crucial to normal development and differentiation of distinct cell lineages in the adult organism. They can be modified by exogenous influences, and, as such, can contribute to or be the result of environmental alterations of phenotype or pathophenotype. Importantly, epigenetic modification can have a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

In an embodiment, the histone methyltransferase inhibitor is an inhibitor of enhancer of zeste homolog 2 (EZH2). EZH2 is a histone-lysine N-methyltransferase enzyme. Non-limiting examples of an EZH2 inhibitor that can be used in the methods provided herein include 3-deazaneplanocin A (DZNep), EPZ6438, EPZ005687 (an S-adenosylmethionine (SAM) competitive inhibitor), EI1, GSK126, and UNC1999. DZNep can inhibit the hydrolysis of S-adenosyl-L-homocysteine (SAH), which is a product-based inhibitor of all protein methyltransferases, leading to increased cellular concentrations of SAH which in turn inhibits EZH2. DZNep may not be specific to EZH2 and can also inhibit other DNA methyltransferases. GSK126 is a SAM-competitive EZH2 inhibitor that has 150-fold selectivity over EZH1. UNC1999 is an analogue of GSK126, and it is less selective than its counterpart GSK126.

In an embodiment, the histone methyltransferase inhibitor is DZNep. In an embodiment, the HDAC inhibitor is a class I HDAC inhibitor, a class II HDAC inhibitor, or a combination thereof. In an embodiment, the HDAC inhibitor is KD5170 (mercaptoketone-based HDAC inhibitor), MC1568 (class IIa HDAC inhibitor), TMP195 (class IIa HDAC inhibitor), or any combination thereof. In some embodiments, HDAC inhibitor is vorinostat, romidepsin (Istodax), chidamide, panobinostat (farydak), belinostat (PXD101), panobinostat (LBH589), valproic acid, mocetinostat (MGCD0103), abexinostat (PCI-24781), entinostat (MS-275), SB939, resminostat (4SC-201), givinostat (ITF2357), quisinostat (JNJ-26481585), HBI-8000, (a benzamide HDI), kevetrin, CUDC-101, AR-42, CHR-2845, CHR-3996, 4SC-202, CG200745, ACY-1215, ME-344, sulforaphane, or any variant thereof.

### Protein Kinase Inhibitors

Aspects of the disclosure relate to the use of protein kinase inhibitors as β cell differentiation factors.

In some embodiments, the protein kinase inhibitor in the methods and composition provided herein comprises staurosporine. In some embodiments, the protein kinase inhibitor in the methods and composition provided herein comprises an analog of staurosporine. Exemplary analogs of staurosporine in the methods and composition provided herein include, without limitation, Ro-31-8220, a bisindolylmaleimide (Bis) compound, 10′-{5″-[(methoxycarbonyl)amino]-2″-methyl}-phenylaminocarbonylstaurosporine, a staralog (see, e.g., Lopez et al., “Staurosporine-derived inhibitors broaden the scope of analog-sensitive kinase technology”, J. Am. Chem. Soc. 2013; 135(48):18153-18159), and, cgp41251.

In some embodiments, the protein kinase inhibitor in the methods and composition provided herein is an inhibitor of PKCβ. In some embodiments, the protein kinase inhibitor in the methods and composition provided herein is an inhibitor of PKCβ with the following structure or a derivative, analogue or variant of the compound as follows:

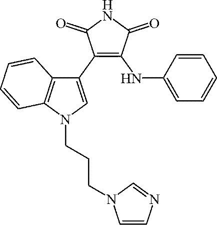 [SMILES: O=C1NC(=O)C(c2cn(CCCn3ccnc3)c3ccccc23)=C1Nc1ccccc1]

In some embodiments, the inhibitor of PKCβ is a GSK-2 compound with the following structure or a derivative, analogue or variant of the compound as follows:

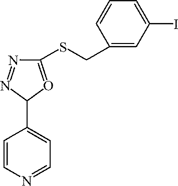

In some embodiments, the inhibitor of PKC in the methods and composition provided herein is a bisindolylmaleimide. Exemplary bisindolylmaleimides include, without limitation, bisindolylmaleimide I, bisindolylmaleimide II, bisindolylmaleimide Ill, hydrochloride, or a derivative, analogue or variant thereof.

In some embodiments, the PKC inhibitor in the methods and composition provided herein is a pseudohypericin, or a derivative, analogue, or variant thereof. In some embodiments, the PKC inhibitor in the methods and composition provided herein is indorublin-3-monoximc, 5-Iodo or a derivative, analogue or variant thereof. In certain embodiments, the methods, compositions, and kits disclosed herein exclude a protein kinase inhibitor.

### Encapsulation Compositions and Devices

In some aspects, the disclosure provides encapsulation compositions or devices comprising any of the cells disclosed herein. Various encapsulation devices, degradable gels and networks can be used for the pharmaceutical compositions of the present disclosure. For example, degradable materials particularly suitable for sustained release formulations include biocompatible polymers, such as poly(lactic acid), poly (lactic-co-glycolic acid), methylcellulose, hyaluronic acid, collagen, and the like.

In some embodiments, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established. Examples of scaffold or matrix (sometimes referred to collectively as “framework”) material that may be used in the present disclosure include nonwoven mats, porous foams, or self-assembling peptides. Nonwoven mats, for example, may be formed using fibers comprising a synthetic absorbable copolymer of glycolic and lactic acids (PGA/PLA), foams, and/or poly(epsilon-caprolactone)/poly(glycolic acid) (PCL/PGA) copolymer.

In some embodiments, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures. In many of the abovementioned cases, the framework may be molded into a useful shape. Furthermore, it will be appreciated that non-native pancreatic β cells may be cultured on pre-formed, nondegradable surgical or implantable devices.

In some embodiments, the matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

In some aspects, the present disclosure provides devices comprising a population of in vitro differentiated cells described herein. In some embodiments, the population of in vitro differentiated cells described herein form cell clusters. A device can be configured to house the cells described herein which, in particular embodiments, produce and release insulin when implanted into a subject. In some embodiments, a device can further comprise one or more semipermeable membrane. The semipermeable membrane can be configured to retain the cell cluster in the device and permit passage of insulin secreted by the cells. In some embodiments of the device, the cells can be encapsulated by the semipermeable membrane. The encapsulation can be performed by any technique available to one skilled in the art. The semipermeable membrane can also be made of any suitable material as one skilled in the art would appreciate and verify. For example, the semipermeable membrane can be made of polysaccharide or polycation. In some embodiments, the semipermeable membrane can be made of poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, biodegradable polyurethanes, albumin, collagen, fibrin, polyamino acids, prolamines, alginate, agarose, agarose with gelatin, dextran, polyacrylates, ethylene- vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, polyethylene oxide, or any combinations thereof. In some embodiments, the semipermeable membrane comprises alginate. In some embodiments, the cells are encapsulated in a microcapsule that comprises an alginate core surrounded by the semipermeable membrane. In some embodiments, the alginate core is modified, for example, to produce a scaffold comprising an alginate core having covalently conjugated oligopeptides with an RGD sequence (arginine, glycine, aspartic acid). In some embodiments, the alginate core is modified, for example, to produce a covalently reinforced microcapsule having a chemoenzymatically engineered alginate of enhanced stability. In some embodiments, the alginate core is modified, for example, to produce membrane-mimetic films assembled by in-situ polymerization of acrylate functionalized phospholipids. In some embodiments, microcapsules are composed of enzymatically modified alginates using epimerases. In some embodiments, microcapsules comprise covalent links between adjacent layers of the microcapsule membrane. In some embodiment, the microcapsule comprises a subsieve-size capsule comprising alginate coupled with phenol moieties. In some embodiments, the microcapsule comprises a scaffold comprising alginate-agarose. In some embodiments, the cells are modified with PEG before being encapsulated within alginate. In some embodiments, the cells are encapsulated in photoreactive liposomes and alginate. It should be appreciated that the alginate employed in the microcapsules can be replaced with other suitable biomaterials, including, without limitation, polyethylene glycol (PEG), chitosan, polyester hollow fibers, collagen, hyaluronic acid, dextran with ROD, BHD and polyethylene glycol-diacrylate (PEGDA), poly(MPC-co-n-butyl methacrylate-co-4-vinylphenyl boronic acid) (PMBV) and poly(vinyl alcohol) (PVA), agarose, agarose with gelatin, and multilayer cases of these. In some embodiments, the device provided herein comprise extracorporeal segment, e.g., part of the device can be outside a subject’s body when the device is implanted in the subject. The extracorporeal segment can comprise any functional component of the device, with or without the cells or cell cluster provided herein.

Further provided herein are methods for treating or preventing a disease in a subject. A composition comprising a population of in vitro differentiated cells described herein can be administered into a subject to restore a degree of pancreatic function in the subject. In some embodiments, such composition is transplanted in a subject. The term “transplant” can refer to the placement of cells or cell clusters, any portion of the cells or cell clusters thereof, any compositions comprising cells, cell clusters or any portion thereof, into a subject, by a method or route which results in at least partial localization of the introduced cells or cell clusters (e.g., within a device) at a desired site. In some embodiments, the desired site is the pancreas. In some embodiments, the desired site is a non-pancreatic location, such as in the liver or subcutaneously or in the preperitoneal region, for example, in a capsule (e.g., microcapsule) to maintain the implanted cells at the implant location and avoid migration. In some embodiments, the transplanted cells release insulin in an amount sufficient for a reduction of blood glucose levels in the subject.

In some embodiments, a composition comprising a population of in vitro differentiated cells described herein are housed in a device that is implanted in a subject. In some embodiments, a composition comprising a population of in vitro differentiated cells described herein are housed in a device suitable for implantation into a subject. In some embodiments, the device upon implantation in a subject releases insulin while retaining the cells in the device and facilitates tissue vascularization in and around the device. Exemplary devices are described, for example in US Pat. No. 11,471,398, US Publication Nos. 2020-0289407, 2021-0016073, 2022-0175511, and PCT Applications WO2018232180, WO2019068059, WO2019178134, WO2020/206150, and WO2020/206157, each of which is incorporated-by-reference in its entirety. In some embodiments, a subject is not administered an immune suppression agent during the implantation or vascularization of the device. In some embodiments, the subject is administered an immune suppression agent (e.g., Thymoglobulin (ATG), sirolimus, Etanercept, tacrolimus, or mycophenolate) for the first 1, 2, 3, 4, 5, 6, or 7 months following the implantation of the device, but the subject is not administered an immune suppression agent (e.g., Thymoglobulin (ATG), sirolimus, Etanercept, tacrolimus, or mycophenolate) 6, 7, 8, 9, 10, 11, 12, 13, 15, 24, or 36 months after the implantation of the device. In some embodiments, the device has a thickness of at least about 300 pm. In some embodiments, the device comprises a membrane comprising a plurality of nodes interconnected by a plurality of fibrils.

In some embodiments, a device of the disclosure may include multiple layers of membranes. At least one exterior membrane of these multiple layers of membranes may be semipermeable. However, embodiments in which each of the membranes is semipermeable or where at least one of the membranes within a device are substantially impermeable are also contemplated. Further, a device may include two stacked membranes, three stacked membranes, and/or any other appropriate number of membranes as the disclosure is not limited in this fashion. For example, in one embodiment including two membranes, either membrane may be semipermeable and the other impermeable or both may be semipermeable. Accordingly, it should be understood that the current disclosure is not limited to any particular combination of membranes within a stacked structure.

In some embodiments, a device may include at least one population of cells disposed within an internal volume of the device. For example, the population of cells may be disposed within an internal volume formed between two or more opposing exterior membranes of the device where an exterior edge of the internal volume may be defined by one or more bonds extended around at least a portion, and in some instances an entire, perimeter of the membranes or other appropriate portion of the membranes. In such an embodiment, at least the exterior membranes of the device may be configured to block passage of the one or more populations of cells out of the device. Accordingly, the one or more populations of cells may be retained within the interior volume of the device. While the use of two exterior membranes forming a single internal volume is noted, the use of multiple intermediate membranes positioned between the exterior membranes of a device and/or multiple unconnected interior volumes within a device are also contemplated. Additionally, instances in which a single membrane is folded over and bonded to itself to provide two opposing membranes to form the interior volume are also contemplated.

To provide the desired selectivity, the porous membranes used with any of the devices disclosed herein may have an open porous structure with average pore sizes that are greater than or equal to about 1 nm, 5 nm, 10 nm, 15 nm, 20 nm, 30 nm, 40 nm, 50 nm, 60 nm, 70 nm, 80 nm, 90 nm, 100 nm, 200 nm, 300 nm, and/or any other appropriate size range. Correspondingly, the average pore size of the various membranes described herein may have an average pore size that is less than or equal to 2500 nm, 2000 nm, 1700 nm, 1500 nm, 1400 nm, 1300 nm, 1200 nm, 1100 nm, 1000 nm, 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, and/or any other appropriate size range. Combinations of the foregoing are contemplated including, for example, an average pore size that is between or equal to 1 nm and 20 nm, 1 nm and 2500 nm, and/or any other appropriate combination. While specific average pore sizes are described above, it should be understood that any appropriate average pore size may be used for the various membranes described herein including average pore sizes both greater than and less than those noted above.

To provide sufficient strength and/or rigidity for a macroencapsulation device, the various membranes and frames may be made from materials that are sufficiently stiff. The desired stiffness may be provided via an appropriate combination of a material’s Young’s modulus (also referred to as an Elastic modulus), thickness, and overall construction which may be balanced with a desired permeability of the device. Appropriate Young’s moduli for the various membranes and frames described herein may be at least 10⁵ Pa, 10⁶ Pa, 10⁷ Pa, 10⁸ Pa, 10⁹ Pa, and/or 10¹⁰ Pa. Other appropriate Young’s moduli for the various membranes and frames described herein may be used including moduli both greater than and less than these ranges. Ranges between the foregoing Young’s moduli are contemplated including, for example, a Young’s modulus between or equal to about 10⁶ Pa and 10¹⁰ Pa.

In some embodiments, it may be desirable for one or more of the membranes included within a macroencapsulation device to be hydrophilic to facilitate loading of cells into the macroencapsulation device and/or to facilitate the flow of one or more fluids, biological compounds, therapeutics, cell nutrients, cell waste, and/or other materials through the membranes of a device. Additionally, a hydrophilic outer membrane may also reduce the occurrence of fibrosis when the device is positioned in vivo. Accordingly, the membranes of a macroencapsulation device may either be made from a hydrophilic material and/or treated with a hydrophilic coating. Appropriate hydrophilic coatings may include, but are not limited to polyhydroxyacrylate, PEG, pHPA, carboxymethylcellulose, alginate, agarose, and/or solute-impregnated thermoplastic coatings. Appropriate hydrophilic materials may also include, but are not limited to an appropriate hydrophilic polymer, polyethylene glycol, polyvinyl alcohol, polydopanine, any combination thereof, and/or any other appropriate hydrophilic material capable of forming a coating on the membranes or that the membranes may be made from.

In some embodiments, the device comprises a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment having a surface area to volume ratio of at least about 40 cm-1, and wherein the enclosed compartment provides a volume for housing a cell within the device.

In some embodiments, the enclosed compartment comprises a single continuous open chamber. In some embodiments, the volume is about 8 µL to about 1,000 µL. In some embodiments, the device has at least one of a length and a width of about 0.25 cm to about 3 cm. In some embodiments, the device has a thickness of at least about 300 pm.

In some embodiments, the plurality of channels is generally perpendicular with respect to the first membrane. In some embodiments, the plurality of channels is arranged in a rectilinear array. In some embodiments, the plurality of channels is arranged in a polar array. In some embodiments, the channel has an average diameter of about 400 pm to about 3,000 pm. In some embodiments, the diameter is measured at a narrowest point in the channel. In some embodiments, a center of each channel is separated from the center of another channel by a distance of about 75 pm to about 500 pm. In some embodiments, the channel has a height to diameter ratio of at least about 0.2. In some embodiments, the device has a number of channels per area along a transverse plane, and in some embodiments the number is greater than about 50 /cm2.

In certain embodiments, it may be desirable to limit a maximum thickness of a device in a direction perpendicular to a plane in which a maximum transverse dimension of the device lies. Accordingly, one or more interior portions of first and second membranes disposed within a frame may be bonded together to limit the extent to which the membranes may be displaced relative to one another. These bonded portions of the membranes may be dispersed uniformly within the interior portion of the membranes located within the frame. These bonded portions may have any appropriate shape including, for example, dots, lines, curves, or any other appropriate shape. While the bonded interior portions may have any appropriate size for a desired application, in one embodiment using bonded dots, the diameter of the bonded dots may be greater than or equal to about 0.5 mm, 0.75 mm, 1 mm, 1.25 mm, 1.5 mm, and/or any other appropriate diameter. Correspondingly, the diameter of the dots may be less than or equal to about 3 mm, 2.75 mm, 2.5 mm, 2.25 mm, 2.0 mm, and/or any other appropriate diameter. Combinations of the above noted ranges are contemplated including, for example, a diameter that is between or equal to 0.5 mm and 3 mm. While specific shapes and size ranges are provided above, it should be understood that other shapes and sizes both smaller and greater than those noted above are contemplated as the disclosure is not limited in this fashion.

In some embodiments, one or more portions of adjacent membranes may be bonded together such that the interior volume within the device is subdivided into a plurality of interconnected channels, which in some embodiments may be shaped like a lumen though any appropriate shape or configuration of the channels may also be used. The channels may have an inner maximum transverse dimension, such as an inner diameter, that is greater than or equal to 40 µm, 50 µm, 100 µm, 200 µm, 300 µm, and/or 400 µm. Correspondingly, the channels may have an inner maximum transverse dimension that is less than or equal to 800 µm, 700 µm, 600 µm, 500 µm, and/or 400 m. Combinations of the foregoing are contemplated including, for example, an inner maximum transverse dimension of the plurality of channels that is between or equal to 40 µm and 800 µm. Further, a density of the interconnected channels forming the various compartments of a device may have a density per unity area within a transverse plane of the device that is greater than or equal to about 10 channels/cm², 15 channels/cm², 20 channels/cm², 25 channels/cm², 30 channels/cm², 35 channels/cm², 40 channels/cm², 45 channels/cm², 50 channels/cm², 60 channels/cm², 70 channels/cm², 80 channels/cm², 90 channels/cm², 100 channels/cm², 110 channels/cm², 120 channels/cm², 130 channels/cm², 140 channels/cm², 150 channels/cm², 175 channels/cm², or 200 channels/cm². Ranges extending between any of the above noted density of channels are also contemplated including, for example, a density of channels that is between or equal to about 10 channels/cm² and 200 channels/cm². Though densities both greater than and less than the ranges described above are also contemplated.

A device as described herein may have any appropriate combination of internal volumes, external dimensions, and/or other appropriate physical parameters. For example, an internal volume encompassed by the outer membranes of a macroencapsulation device may be between or equal to 40 µL and 250 µL. A width, or maximum transverse dimension, of the macroencapsulation device may also be between about 20 mm and 80 mm. Additionally, to provide a desired diffusion of oxygen into the interior of a macroencapsulation device to support cells contained therein, a maximum oxygen diffusion distance from an exterior of the device to an interior portion of the device including a population of cells may be less than 50 µm, 100 µm, 150 µm, 200 µm, 250 µm, 300 µm, 350 µm, 400 µm, 450 µm, or 500 µm. In some embodiments, the maximum oxygen diffusion distance from an exterior of the device to an interior portion of the device including a population of cells is less than or equal to 150 µm. In some embodiments, the maximum oxygen diffusion distance from an exterior of the device to an interior portion of the device including a population of cells is less than or equal to 200 µm. In some embodiments, the maximum oxygen diffusion distance from an exterior of the device to an interior portion of the device including a population of cells is less than or equal to 250 µm. Correspondingly, a maximum thickness, or dimension perpendicular to a maximum transverse dimension, of the overall device and/or an internal volume located within the device may be less than 50 µm, 100 µm, 150 µm, 200 µm, 250 µm, 300 µm, 350 µm, 400 µm, 450 µm, or 500 µm. In some embodiments, the maximum thickness, or dimension perpendicular to a maximum transverse dimension, of the overall device and/or an internal volume located within the device is less than or equal to 500 µm. Further, in some embodiments, an outer surface area to volume ratio of the device may be greater than or equal to about 20 cm⁻¹, 40 cm⁻¹, 60 cm⁻¹, 80 cm⁻¹, 100 cm⁻¹, 120 cm⁻¹, or 150 cm⁻¹. Ranges extending between any of the forgoing values for the various dimensions and parameters as well as ranges both greater than and less those noted above are also contemplated.

In some embodiments, at least one of the first membrane and the second membrane comprise a plurality of nodes interconnected by a plurality of fibrils. In some embodiments, at least one of the first membrane and the second membrane comprise PVDF, PTFE, ePTFE, PCL, PE/PES, PP, PS, PMMA, PLGA, PLLA, or any combination thereof. In some embodiments, the device further comprises an opening through the first membrane and/or the second membrane within the channel. In some embodiments, the opening has a concentricity with respect to the channel of at most about 25% the diameter of the channel. In some embodiments is a frame configured to receive the device described herein. In some embodiments, the frame is configured to receive a plurality of cell housing devices. In some embodiments, the frame comprises a flexing mechanism configured to prevent buckling of the cell housing device.

In some embodiments, the device comprises a porous polymeric membrane including a plurality of open pores, wherein the open pores have an average pore size equal to or less than 1 um, wherein said membrane has an average thickness of between 10 um and 150 um, and wherein said membrane has a tensile strength of at least 1 MPa; and a population of cells, wherein said population of cells comprises at least one selected from the group of pancreatic progenitor cells, endocrine cells, alpha cells, delta cells, and beta cells, wherein said membrane is configured to exhibit a ratio of Dfirst/Dsecond equal to or greater than 2 and less than or equal to 50, wherein Dfirst is a first diffusion coefficient for a first molecule having a first molecular weight between or equal to 50 Da and 10 kDa, wherein Dsecond is a second diffusion coefficient for a second molecule having a second molecular weight between or equal to 50 kDa and 500 kDa, and wherein a ratio of said second molecular weight to said first molecular weight is equal to or greater than 10. In some embodiments, the device comprises a first membrane having a first surface and a second surface opposing the first surface; and a second membrane opposite and attached to the second surface of the first membrane; wherein the first membrane and the second membrane form an enclosed compartment configured to house a cell population, and wherein at least one of the first membrane and the second membrane is sintered. In some embodiments, both the first membrane and the second membrane are sintered. Sintering of a membrane may be used to alter the porosity and flux properties of a membrane. For example, the sintering may increase the porosity of the membrane while maintaining its pore structure. The sintering may also improve the mechanical stability and diffusive flux of the membrane. Thus, sintering may be used to alter the porosity and/or mechanical properties of the membranes, which in turn can be used to tune the porosity and the flux properties of the macroencapsulation device. Accordingly, in some embodiments, any desired combination of sintered and/or unsintered membranes may be used. For instance, two exterior membranes of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

In some embodiments, an implantable encapsulation device comprises an internal volume comprising, disposed therein, a population of in vitro differentiated cells or a composition comprising a population of in vitro differentiated cells described herein described herein. In some embodiments, the implantable encapsulation device comprises at least one membrane that at least partially defines the internal volume. In some embodiments, the at least one membrane includes a first membrane and a second membrane, wherein the first membrane and the second membrane are bonded together to form a seal extending at least partially around the internal volume disposed between the first membrane and the second membrane. In some embodiments, the at least one membrane comprises at least one selected from PVDF, PTFE, ePTFE, PCL, PE/PES, PP, PS, PMMA, PLGA, and PLLA. In some embodiments, the at least one membrane comprises ePTFE.

In some embodiments, during a manufacturing process of a - 163 -icroencapsulation device, at least one, and in some instances at least two, or more flexible membranes of the device may be deformed to fit at least partially within and subsequently bonded to a frame to form the - 163 -icroencapsulation device. The frame may hold the membranes in the desired configuration where the membranes have a desired amount of slack extending between opposing portions of the frame. For example, in one embodiment, an outer perimeter of a first membrane and a second membrane disposed on the first membrane may be deformed from a first maximum transverse dimension to a second maximum transverse dimension smaller than the first maximum transverse dimension prior to bonding with a frame. This deformation and subsequent holding of the membranes in the deformed configuration may cause the membranes to be held in the frame with a desired amount of slack to accommodate the excess material contained within the frame and may be accomplished in a number of different ways.

The membranes described in the various embodiments of macroencapsulation devices described herein may be bonded to one another using any appropriate bonding method as the disclosure is not limited in this fashion. For example, adjacent membranes may be bonded to one another using an adhesive, an epoxy, a weld or other fusion based technique (e.g. ultrasonic bonding, laser bonding, physical bonding, thermal bonding, etc.), mechanical clamping using a frame or fixture, and/or any other appropriate bonding method. In one specific embodiment, adjacent membranes may be bonded using a heated tool that is used to press or strike two or more membranes against each other for a set fusion time with a predetermined pressure and/or force. In view of the above, it should be understood that the current disclosure is not limited to the use of any particular method for bonding the membranes together.

In some embodiments, the disclosure provides for a device comprising at least 50 x 10⁶ cells. In some embodiments, the disclosure provides for a device comprising no more than 800 x 10⁶ cells. In some embodiments, the disclosure provides for a device comprising between 40 x 10⁶ cells and 100 x 10⁶ cells, between 50 x 10⁶ cells and 90 x 10⁶ cells, between 60 x 10⁶ cells and 80 x 10⁶ cells, or between 70 x 10⁶ cells and 80 x 10⁶ cells. In some embodiments, the device comprises about 75 x 10⁶ cells. In some embodiments, between 10 x 10⁶ and 60 x 10⁶, between 10 x 10⁶ and 50 x 10⁶, between 10 x 10⁶ and 40 x 10⁶, between 10 x 10⁶ and 30 x 10⁶, between 25 x 10⁶ and 60 x 10⁶, or between 25 x 10⁶ and 40 x 10⁶ of the cells in the device are NKX6.1-positive, ISL1-positive cells. In some embodiments, the disclosure provides for the administration to a patient one or more devices, wherein the one or more devices comprise at least 50 x 10⁶ cells. In some embodiments, the disclosure provides for the administration to a patient one or more devices, wherein the one or more devices comprise no more than 120 x 10⁷ cells. In some embodiments, the disclosure provides for the administration to a patient one or more devices, wherein the one or more devices comprise between 50 x 10⁶ cells and 120 x 10⁷ cells, 50 x 10⁶ cells and 500 x 10⁶ cells, 50 x 10⁶ cells and 300 x 10⁶ cells, 100 x 10⁶ cells and 900 x 10⁶ cells, between 100 x 10⁶ cells and 600 x 10⁶ cells, between 100 x 10⁶ cells and 500 x 10⁶ cells, between 250 x 10⁶ cells and 900 x 10⁶ cells, between 250 x 10⁶ cells and 500 x 10⁶ cells, between 350 x 10⁶ cells and 900 x 10⁶ cells, between 350 x 10⁶ cells and 500 x 10⁶ cells, between 400 x 10⁶ cells and 500 x 10⁶ cells, between 150 x 10⁶ cells and 700 x 10⁶ cells, between 130 x 10⁶ cells and 470 x 10⁶ cells, between 450 x 10⁶ cells and 900 x 10⁶ cells, between 800 x 10⁶ cells and 1000 x 10⁶ cells, or between 450 x 10⁶ cells and 600 x 10⁶ cells. In particular embodiments,, the disclosure provides for the administration to a patient one or more devices, wherein the one or more devices comprise between 250 x 10⁶ cells and 500 x 10⁶ cells. In some embodiments, between 50 x 10⁶ and 800 x 10⁶, between 50 x 10⁶ and 400 x 10⁶, between 50 x 10⁶ and 300 x 10⁶, between 50 x 10⁶ and 800 x 10⁶, between 50 x 10⁶ and 200 x 10⁶, between 150 x 10⁶ and 800 x 10⁶, between 150 x 10⁶ and 500 x 10⁶, between 50 x 10⁶ and 250 x 10⁶, between 200 x 10⁶ and 800 x 10⁶, between 200 x 10⁶ and 500 x 10⁶, between 200 x 10⁶ and 300 x 10⁶, between 300 x 10⁶ and 800 x 10⁶, or between 500 x 10⁶ and 800 x 10⁶ of the cells in the one or more devices are NKX6.1-positive, ISL1-positive cells. In particular embodiments, between 150 x 10⁶ and 250 x 10⁶ of the cells in the one or more devices are NKX6.1-positive, ISL1-positive cells. In some embodiments, the disclosure provides for the administration to a patient 1, 2, 3, 4, 5, 6, 7, 8, 9, or 10 devices. In some embodiments, the disclosure provides for the administration to a patient 2, 4, 6 or 8 devices. In some embodiments, the disclosure provides for the administration of 4 devices. In some embodiments, the disclosure provides for the administration of 6 devices. In some embodiments, the disclosure provides for the administration of 2-8, 3-7, or 4-6 devices.

The cell compositions in any of the devices disclosed herein comprise one or more pharmaceutically acceptable excipients. In some embodiments, the pharmaceutically acceptable carrier is a surfactant. In some embodiments, the pharmaceutically acceptable carrier is a buffer. In some embodiments, the pharmaceutically acceptable excipient is DMEM. In some embodiments, the pharmaceutically acceptable excipient is DMEM/F12. In some embodiments, the pharmaceutically acceptable excipient comprises human serum albumin.

In some embodiments, any of the devices disclosed herein are administered to a subject, e.g., via implantation. In some emodiments, any of the devices disclosed herein are administered to a subject extrahepatically. In some embodiments, one or more devices are administered subcutaneously. In some embodiments, one or more devices are administered subabdominally. In some embodiments, one or more devices are administered peritoneally. In some embodiments, one or more devices are administered preperitoneally. In some embodiments, one or more devices are administered into the preperitoneal space of the abdominal wall. In some embodiments, one or more devices are administered into the preperitoneal space of the anterior abdominal wall.

In some embodiments, the device is filled with cells to completion, which permits volumetric dose calculation based on the volume capacity of the device. For example, the device volume may be calculated based on extrapolation from a solid model using individual measurements of, for example, the fusion dots to account for the un-fillable area of the device. Second, the volume of each individual cell in the drug substance may be assessed using an automated cell counting instrument that outputs the average diameter. Third, the packing fraction of cells during encapsulation may be estimated to a specific percentage based on empirical testing (e.g., 80%, 80%, 90%, or, in some embodiments, 88%). Together, these measurements may be used to calculate the number of cells that are filled into the device by dividing the volume of the device by the volume of each individual cell adjusted by a maximum capacity of, for example, 88%.

In some embodiments, following encapsulation, an assessment of the number of total cells loaded into the device is required for dose calculation (this calculation may be referred to as DCN, or device cell number). In some embodiments, internal device volume of the device is calculated for each encapsulated device, analogous to calculation of packed cell volume as assessed in clinical islet infusions. Device cell number may be determined from two primary inputs, the internal device volume available to hold cells (device volume) and the volume of the individual cells being encapsulated (cell volume).

In some embodiments, device volume may be assessed in-silico from manufacturing specifications, including slack (the relationship of the devices PEEK frame inner diameter, and the membrane outer diameter held constant during device manufacturing) in addition to fusion diameter (membrane bonded between individual channels) of the device (e.g., any of the devices disclosed herein). Cell volume is calculated from cell radii, which may be reported from the cell count and viability assessment generated from a LUNA FX Cell counter with each individual cell prep that is loaded to the device during encapsulation proceedings.

In some embodiments, the Device Cell Number (DCN) assessment is a volumetric calculation to measure the number of cells loaded into a device at a constant packing fraction (PF) as a function of Device and Cell Volume.

$$D C N = P F \times \frac{V o l u m e {}_{d e v i c e}}{V o l u m {e}_{c e l l}} \times 2 D F i l l \%$$

KIT

In some aspects, the disclosure provides a kit. For example, the disclosure provides a kit for the treatment, amelioration and/or prevention of disease characterized by high blood sugar levels over a prolonged period of time, e.g., diabetes, e.g., Type 1 diabetes or Type 2 diabetes in a subject at risk for developing such a disease. In some embodiments, the kit includes (a) a pharmaceutical composition described herein, and, optionally (b) informational material. In some embodiments, the kit includes (a) a population of cells in a liquid suspension described herein, and, optionally (b) informational material. In some embodiments, the disclosure provides for a container housing any of the devices and cell compositions disclosed herein. In some embodiments, the container comprises a media in which the device and cell compositions are bathed. The informational material may be descriptive, instructional, marketing or other material that relates to the methods described herein and/or the use of the pharmaceutical composition for the methods described herein. The pharmaceutical composition may comprise material for a single administration (e.g., single dosage form), or may comprise material for multiple administrations (e.g., a “multidose” kit).

The informational material of the kits is not limited in its form. In one embodiment, the informational material may include information about production of the pharmaceutical composition, date of expiration, batch or production site information, and so forth. In one embodiment, the informational material relates to methods for administering a dosage form of the pharmaceutical composition.

In addition to a dosage form of the pharmaceutical composition described herein, the kit may include other ingredients, such as a second agent for treating a disease described herein. Alternatively, the other ingredients may be included in the kit, but in different compositions or containers than a pharmaceutical composition described herein. In such embodiments, the kit may include instructions for admixing a pharmaceutical composition described herein and the other ingredients, or for using a pharmaceutical composition described herein together with the other ingredients.

In some cases, the kit disclosed herein further comprises an immune response modulator disclosed herein, e.g., for reducing transplant rejection by the subject against the infused cells. In some embodiments, the immune response modulator is not or does not comprise a steroid. In some cases, the immune response modulator comprises a steroid such as corticosteroid. Examples of immune response modulator that can be used in the methods can include purine synthesis inhibitors like azathioprine and mycophenolic acid, pyrimidine synthesis inhibitors like leflunomide and teriflunomide, antifolate like methotrexate, tacrolimus, ciclosporin, pimecrolimus, abetimus, gusperimus, lenalidomide, pomalidomide, thalidomide, PDE4 inhibitor, apremilast, anakinra, sirolimus, everolimus, ridaforolimus, temsirolimus, umirolimus, zotarolimus, anti-thymocyte globulin antibodies, anti-lymphocyte globulin antibodies, CTLA-4, fragment thereof, and fusion proteins thereof like abatacept and belatacept, TNF inhibitor like etanercept and pegsunercept, aflibercept, alefacept, rilonacept, antibodies against complement component 5 like eculizumab, anti-TNF antibodies like adalimumab, afelimomab, certolizumab pegol, golimumab, infliximab, and nerelimomab, antibodies against Interleukin 5 like mepolizumab, anti-Ig E antibodies like omalizumab, anti-Interferon antibodies like faralimomab, anti-IL-6 antibodies like elsilimomab, antibodies against IL-12 and IL-23 like lebrikizumab and ustekinumab, anti-IL-17A antibodies like secukinumab, anti-CD3 antibodies like muromonab-CD3, otelixizumab, teplizumab, and visilizumab, anti-CD4 antibodies like clenoliximab, keliximab, and zanolimumab, anti-CD11a antibodies like efalizumab, anti-CD18 antibodies like erlizumab, anti-CD20 antibodies like obinutuzumab, rituximab, ocrelizumab and pascolizumab, anti-CD23 antibodies like gomiliximab and lumiliximab, anti-CD40 antibodies like teneliximab and toralizumab, antibodies against CD62L/L-selectin like aselizumab, anti-CD80 antibodies like galiximab, anti-CD147/Basigin antibodies like gavilimomab, anti-CD154 antibodies like ruplizumab, anti-BLyS antibodies like belimumab and blisibimod, anti-CTLA-4 antibodies like ipilimumab and tremelimumab, anti-CAT antibodies like bertilimumab, lerdelimumab, and metelimumab, anti-Integrin antibodies like natalizumab, antibodies against Interleukin-6 receptor like tocilizumab, anti-LFA-1 antibodies like odulimomab, antibodies against IL-2 receptor/CD25 like basiliximab, daclizumab, and inolimomab, antibodies against T-lymphocyte (Zolimomab aritox) like atorolimumab, cedelizumab, fontolizumab, maslimomab, morolimumab, pexelizumab, reslizumab, rovelizumab, siplizumab, talizumab, telimomab aritox, vapaliximab, and vepalimomab, antibodies against CD52 like alemtuzumab, blockers of inosine monophosphate dehydrogenase (IMPDH) like mycophenolate mofetil, inhibitors of cell emigration like FTY720. In some embodiments, the immune response modulator is selected from the group consisting of: Thymoglobulin, Etanercept, Basiliximab, Tacrolimus, Sirolimus, and Mycophenolate mofetil, or any combination thereof.

The kit may include one or more containers for the composition containing a dosage form described herein. In some embodiments, the kit contains separate containers, dividers or compartments for the composition and informational material. For example, the pharmaceutical composition may be contained in a bottle, vial, or syringe, and the informational material may be contained in a plastic sleeve or packet. In other embodiments, the separate elements of the kit are contained within a single, undivided container. For example, the dosage form of a pharmaceutical composition described herein is contained in a bottle, vial or syringe that has attached thereto the informational material in the form of a label.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1. Infusion of Non-Native Pancreatic Cells to Treat Diabetes in Human

### Subject

This example illustrates treatment of human subjects with Type I diabetes by infusion of non-native pancreatic cells (also termed as “stem cell-derived islet cells” or “SC-islet cells” hereafter) according to some embodiments of the present disclosure. The following clinical efficacy and safety data indicate that the exemplary formulation was well tolerated by the human subject and led to robust improvements in glycemic control and restoration of islet cell function.

SC-islet cells, including mature, non-native β cells capable of releasing insulin in response to glucose challenge in vitro, were generated by an in vitro differentiation process. The differentiation process followed a 6-stage stepwise protocol, starting from human embryonic stem cells to generation of mature β cells, as outlined below in Table 3. Following Stage 5 (S5) cells were disassociated and cryopreserved. The cryopreserved cells were thawed prior to stage 6 (S6).

TABLE 3

| | |
|---|---|
| S1D1 | 0.05% HSA |
| | 250 µM L-ascorbic acid |
| | 7.7 nM Activin-A |
| | 3 mM CHIR99021 |
| SID2 | 0.05% HSA |
| | 250 µM L-ascorbic acid |
| | 7.7 nM Activin-A |
| S1D3 | 0.05% HSA |
| | 250 µM L-ascorbic acid |
| | 7.7 nM Activin-A |
| S2D1 | 0.05% HSA |
| | 250 µM L-ascorbic acid |
| | 2.6 nM KGF |
| S2D2 | 0.05% HSA |
| | 250 µM L-ascorbic acid |
| | 2.6 nM KGF |
| S2D3 | 0.05% HSA |
| | 250 µM L-ascorbic acid |
| | 2.6 nM KGF |
| S3D1 | 0.05% HSA |
| | 250 µM L-ascorbic acid |
| | 2.6 nM KGF |
| | 250 nM SANT-1 |
| | 2 mM Retinoic Acid |
| | 500 nM PDBU |
| | 2.5 µM Thiazovivin |
| | 250 nM DMH-1 |
| | 1.5 nM Activin-A |
| S3D2 | 0.05% HSA |
| | 250 µM L-ascorbic acid |
| | 2.6 nM KGF |
| | 250 nM SANT-1 |
| | 2 mM Retinoic Acid |
| | 500 nM PDBU |
| | 2.5 µM Thiazovivin |
| S4D1 | 0.05% HSA |
| | 250 µM L-ascorbic acid |
| | 2.6 nM L KGF |
| | 250 nM SANT-1 |
| | 100 nM Retinoic Acid |
| | 2.5 µM Thiazovivin |
| | 0.4 nM Activin-A |
| S4D2 | No Feed |
| S4D3 | 0.05% HSA |
| | 250 µM L-ascorbic acid |
| | 2.6 nM KGF |
| | 250 nM SANT-1 |
| | 100 nM Retinoic Acid |
| | 2.5 µM Thiazovivin |
| | 0.4 nM Activin-A |
| S5D4 | No Feed |
| S4D5 | 0.05% HSA |
| | 250 µM L-ascorbic acid |
| | 2.6 nM KGF |
| | 250 nM SANT-1 |
| | 100 nM Retinoic Acid |
| | 2.5 µM Thiazovivin |
| | 0.4 nM Activin-A |
| S4D6 | No Feed |
| S5D1 | 0.05% HSA |
| | 250 µM L-ascorbic acid |
| | 250 nM SANT-1 |
| | 2.2 nM Betacellulin |
| | 2 mM XXI |
| | 100 nM LDN |
| | 10 mM ALK5 Inhibitor II |
| | 1 mM GC 1 |
| | 2.5 µM Thiazovivin |
| | 3 nM SSP |
| | 100 nM DZNEP |
| | 50 nM Retinoic Acid |
| | 10 µM ZnSO₄ |
| S5D2 | 0.05% HSA |
| | 250 µM L-ascorbic acid |
| | 250 nM SANT-1 |
| | 2.2 nM Betacellulin |
| | 2 mM XXI |
| | 100 nM LDN |
| | 10 mM ALK5 Inhibitor II |
| | 1 mM GC 1 |
| | 2.5 µM Thiazovivin |
| | 3 nM SSP |
| | 100 nM DZNEP |
| | 50 nM Retinoic Acid |
| | 10 µM ZnSO₄ |
| S5D3 | No Feed |
| S5D4 | 0.05% HSA |
| | 250 µM L-ascorbic acid |
| | 2 mM XXI |
| | 100 nM LDN |
| | 10 mM ALK5 Inhibitor II |
| | 1 mM GC 1 |
| | 2.5 µM Thiazovivin |
| | 3 nM SSP |
| | 100 nM DZNEP |
| | 10 µM ZnSO₄ |
| S5D5 | No Feed |
| S5D6 | 0.05% HSA |
| | 250 µM L-ascorbic acid |
| | 2 mM XXI |
| | 100 nM LDN |
| | 10 mM ALK5 Inhibitor II |
| | 1 mM GC 1 |
| | 2.5 µM Thiazovivin |
| S6D1-3 | 0.05% HSA 250 uM L-ascorbic acid 100 nM LDN-193189 10 uM ALK5 Inhibitor II 1 uM GC-1 2.5 µM Thiazovivin 3 nM Staurosporine 100 nM DZNEP |
| S6D4 | No Feed, supplement with Human Serum Albumin, 20% |
| S6D4-5 | 0.05% HSA 250 uM L-ascorbic acid |
| S6D6 | No feed, supplement with Human Serum Albumin, 20% |

Infusion doses were each prepared as a sterile cell suspension in HypoThermosol® in a gas-permeable bag, containing about 4×10⁸ total SC-islet cells suspended in 200 mL of HypoThermosol® FRS.

In this study, three subjects received the SC-islet compositions: Subject A1, Subject A2/B2, and Subject B2.

Subject A1 was a 64-year-old age male diagnosed with T1D at the age of 22 years, with history of impaired hypoglycemia awareness and SHEs (3 events reported in the 1 year before screening and an additional 2 events during the screening period). At time of screening, the subject presented with no residual endogenous β-cell function as indicated by undetectable C-peptide (imputed as 7 pmol/L [0.02 ng/mL]) during a 4-hour MMTT. The subject’s baseline HbA1c was 8.6%, and baseline total daily insulin dose was 34 U/day.

Subject A2/B2 was a 35-year-old female diagnosed with T1D at the age of 24 years, with history of impaired hypoglycemia awareness and SHEs (3 events reported in the 1 year before screening). At time of screening, the subject presented with no residual endogenous β-cell function as indicated by undetectable C-peptide (imputed as 7 pmol/L [0.02 ng/mL]) during a 4-hour MMTT. The subject’s baseline HbA1c was 7.5%, and baseline total daily insulin dose was 25.9 U/day.

Subject B1 was a 46-year-old male diagnosed with T1D at the age of 27 years, with a history of impaired glycemia awareness and SHEs (2 events in the 1 year before screening). At time of screening, the subject presented with no residual endogenous β-cell function as indicated by undetectable C-peptide (imputed as 7 pmol/L [0.02 ng/mL]) during a 4-hour MMTT. The subject’s baseline HbA1c was 7.6%, and baseline total daily insulin dose was 45.1 U/day.

Subject A1 received 1 infusion of VX-880 at 0.4 × 10⁹ total SC-islet cells. Prior to infusion, Subject A1 received Thymoglobulin (ATG) and sirolimus. Subject A1 was also on Etanercept. Post-infusion, Subject A1 received a dose of ATG on days 2 and 3 and received tacrolimus along with sirolimus or mycophenolate as maintenance immunosuppression throughout the study.

Subject A2 initially received an infusion of 0.4 × 10⁹ total SC-islet cells in “Part A.” Subject A2 subsequently received a second infusion 0.4 × 10⁹ total SC-islet cells and was enrolled in “Part B” as Subject B2, and so the subject is referred to as “Subject A2/B2.” Prior to infusion, Subject A2/B2 received Thymoglobulin (ATG) and sirolimus. Subject A2/B2 also received Etanercept around the infusion. Post-infusion, Subject A2/B2 received a dose of ATG on days 2 and 3 and received tacrolimus and sirolimus as maintenance immunosuppression throughout the study.

Subject B1 received 1 infusion of 0.8 × 10⁹ total SC-islet cells. Infusions were administered via the hepatic portal vein. Prior to infusion, Subject B1 received Thymoglobulin (ATG) and sirolimus. Subject B1 also received Etanercept around the infusion. Post-infusion, Subject B1 received a dose of ATG on days 2 and 3 and received tacrolimus and sirolimus as maintenance immunosuppression throughout the study.

As of the data cut-off date, Subject A1 completed follow-up visits after VX-880 infusion through Month 15, and A2/B2 completed follow-up visits after Day 29 after second infusion (second infusion occurred on Day 269 after the first infusion). Subject B1 completed visits through Day 180 after SC-islet infusion.

### C-Peptide and Glucose Response to MMTT

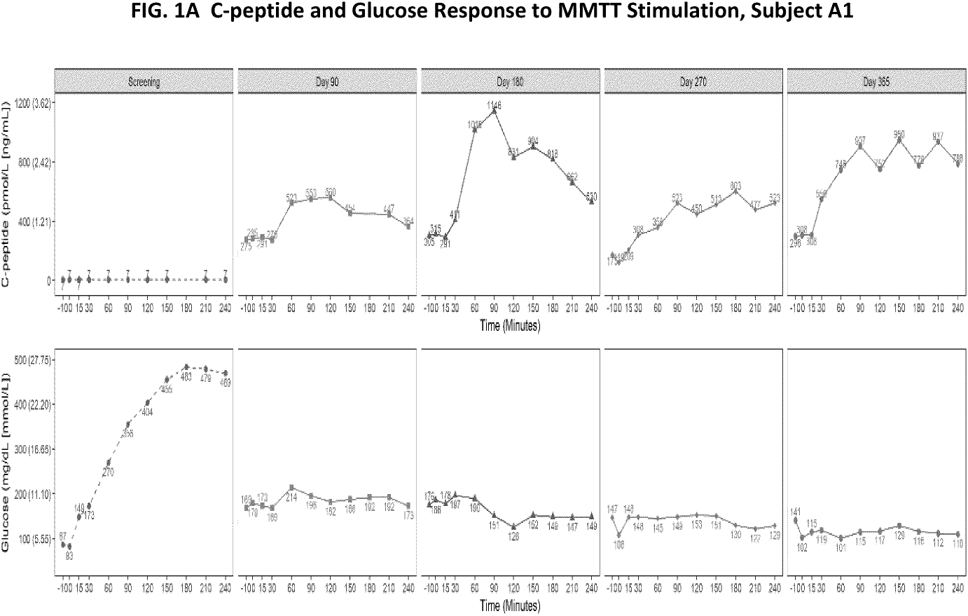

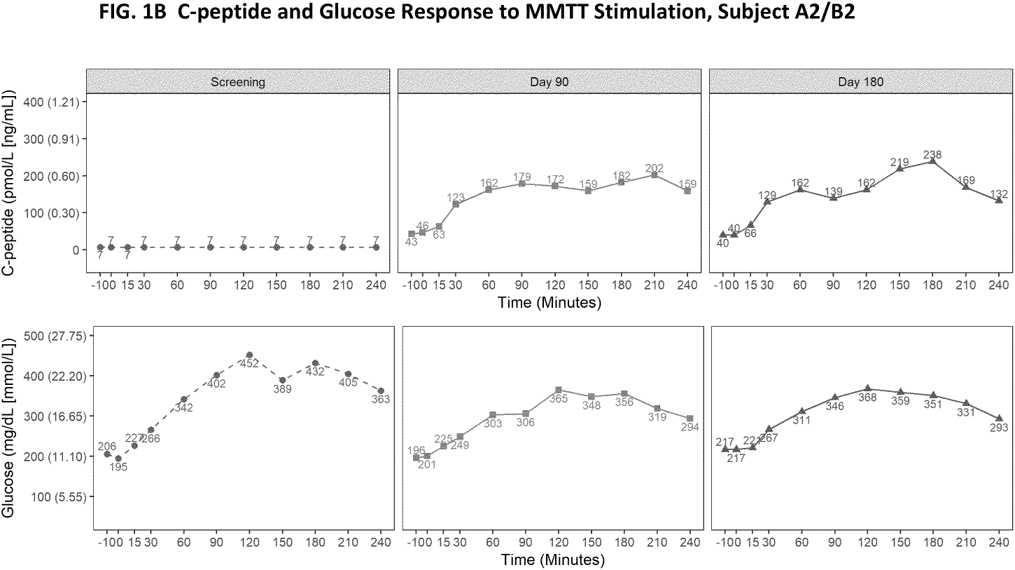

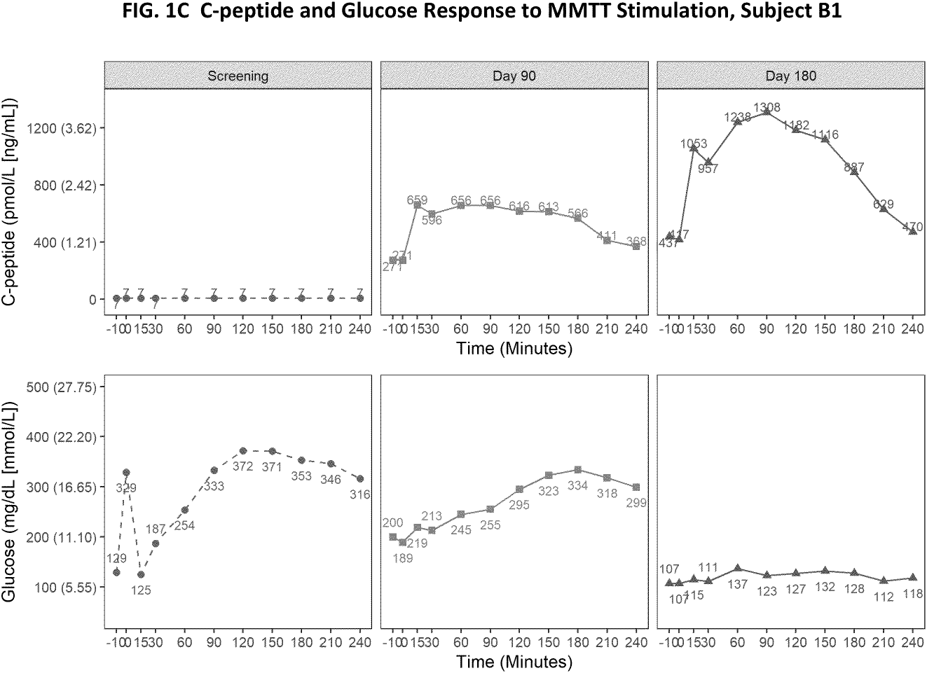

Islet cell function was evaluated at Screening and specified timepoints after infusion using a mixed meal tolerance test (MMTT) as shown in FIG. 1A, FIG. 1B and FIG. 1C. At Screening, fasting and stimulated C-peptide, a marker of endogenous insulin secretion, were below the limit of detection in all 3 subjects, indicating no endogenous insulin production. At Day 90, all 3 subjects had substantial, clinically meaningful improvements in MMTT-stimulated peak C-peptide ≥200 pmol/L (0.60 ng/mL), with concomitant reductions in glycemic excursions, indicating significant endogenous insulin secretion (FIG. 1A, FIG. 1B and FIG. 1C). Further increases in MMTT-stimulated peak C-peptide levels >1000 pmol/L (3.02 ng/mL) were seen in Subject A1 and Subject B1 at the Day 180 assessments (FIG. 1A and FIG. 1B); Day 180 was the last MMTT assessment for Subject B1 as of the data cut-off date. For Subject A1, the significant increase in MMTT-stimulated peak C-peptide levels compared to baseline and reduction in glycemic excursions were sustained through Day 365 (last MMTT assessment as of the data cut-off date (FIG. 1A).

### Subject A1

During the MMTT performed at Screening before SC-islet infusion, fasting and stimulated C-peptide were below the limit of detection, and glucose levels exceeded 400 mg/dL (22.2 mmol/L) at multiple timepoints, with a peak glucose level of 483 mg/dL (26.8 mmol/L). At the Day 90 MMTT, fasting C-peptide level was 280 pmol/L (0.85 ng/mL) with a fasting glucose level of 174 mg/dL (9.7 mmol/L). C-peptide levels increased after MMTT stimulation to a peak of 560 pmol/L (1.69 ng/mL), with concomitant glucose levels not exceeding 214 mg/dL (11.9 mmol/L) for the duration of the 4-hour MMTT assessment. Further improvements in fasting and stimulated C-peptide levels with concomitant reductions in stimulated glucose levels were also observed at Day 180 during which peak C-peptide was 1146 pmol/L (3.46 ng/mL) with concomitant glucose levels not exceeding 197 mg/dL (10.9 mmol/L) for the duration of the 4-hour MMTT assessment and remaining persistently below 160 mg/dL (8.9 mmol/L) from 90 to 240 min (FIG. 1A). Notably, the reduction in glucose levels observed during the Day 90 MMTT was reflective of endogenous insulin production; the subject did not administer any exogenous insulin for 5 consecutive days in the week before the Day 90 MMTT and did not administer any exogenous insulin on the day before and day of the Day 90 MMTT. At Day 180, the subject’s mean daily dose of exogenous insulin was only 1.4 U/day during the week before the MMTT was performed. At Day 270 and sustained at Day 365, Subject A1 was deemed to meet the criteria for insulin independence, i.e., able to titrate off insulin therapy for at least 1 week, HbA1c ≤7%, post-prandial serum glucose ≤180 mg/dL (10.0 mmol/L) at 90 minutes during MMTT, fasting serum glucose ≤126 mg/dL (7.0 mmol/L) during MMTT (at either -10 minutes or 0 minutes), and at least 1 MMTT fasting or stimulated C-peptide ≥166 pmol/L (0.50 ng/mL).

### Subject A2/B2

During the MMTT performed at screening before SC-islet infusion, fasting and stimulated C-peptide were below the limit of detection, and glucose levels exceeded 400 mg/dL (22.2 mmol/L) at multiple timepoints, with a peak glucose level of 452 mg/dL (25.1 mmol/L). At the Day 90 after the first infusion MMTT, fasting C-peptide level was 45 pmol/L (0.14 ng/mL) with a fasting glucose level of 199 mg/dL (11.0 mmol/L). C-peptide increased after MMTT stimulation to a peak of 202 pmol/L (0.61 ng/mL). Concomitant glucose levels did not exceed 365 mg/dL (20.3 mmol/L) for the duration of the 4-hour MMTT assessment (FIG. 1B). Similar fasting and stimulated C-peptide and glucose levels were observed at Day 180 after the first infusion. Subject A2/B2 received a second infusion of SC-islets on Day 269 after the first infusion and has been followed through Day 29 after the second infusion. As of the data cut-off date, MMTT was not performed after the second infusion.

### Subject B1

During the MMTT performed at Screening before SC-islet infusion, fasting and stimulated C-peptide were below the limit of detection, and glucose levels exceeded 350 mg/dL (19.4 mmol/L) at several timepoints, with a peak glucose level of 372 mg/dL (20.6 mmol/L). At the Day 90 MMTT, the fasting C-peptide level was 271 pmol/L (0.82 ng/mL) with a fasting glucose level of 195 mg/dL (10.8 mmol/L). C-peptide levels increased after MMTT stimulation to a peak of 659 pmol/L (1.99 ng/mL), with concomitant glucose levels not exceeding 334 mg/dL (18.5 mmol/L) for the duration of the 4-hour MMTT assessment (FIG. 1C). At the Day 180 MMTT, the fasting C-peptide level was 427 pmol/L (1.29 ng/mL), with a fasting glucose level of 107 mg/dL (5.9 mmol/L). C-peptide increased after MMTT stimulation to a peak of 1308 pmol/L (3.95 ng/mL). Concomitant glucose levels did not exceed 137 mg/dL (7.6 mmol/L) for the duration of the 4-hour MMTT assessment. At Day 180, Subject B1 was deemed to meet the criteria insulin independence.

### Changes In HbA1c Over Time

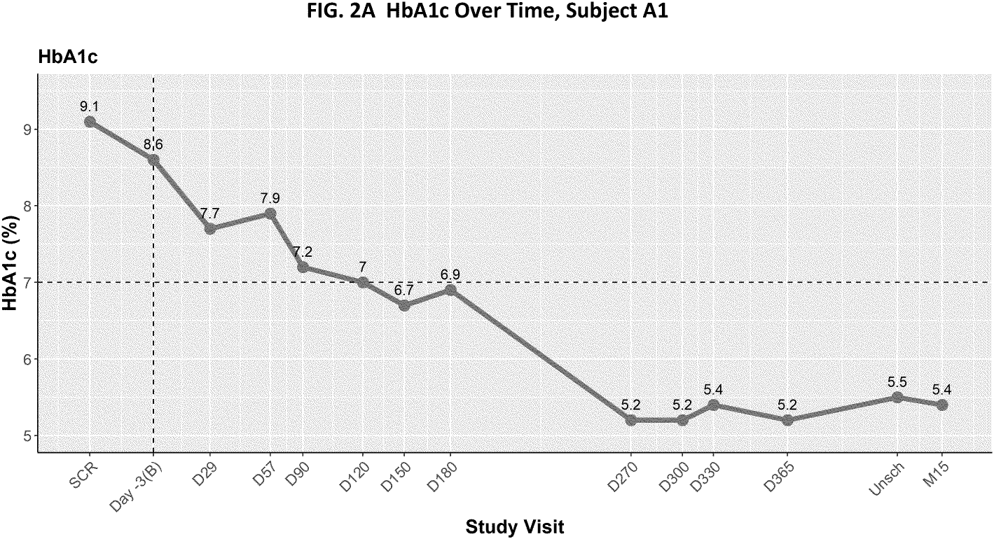

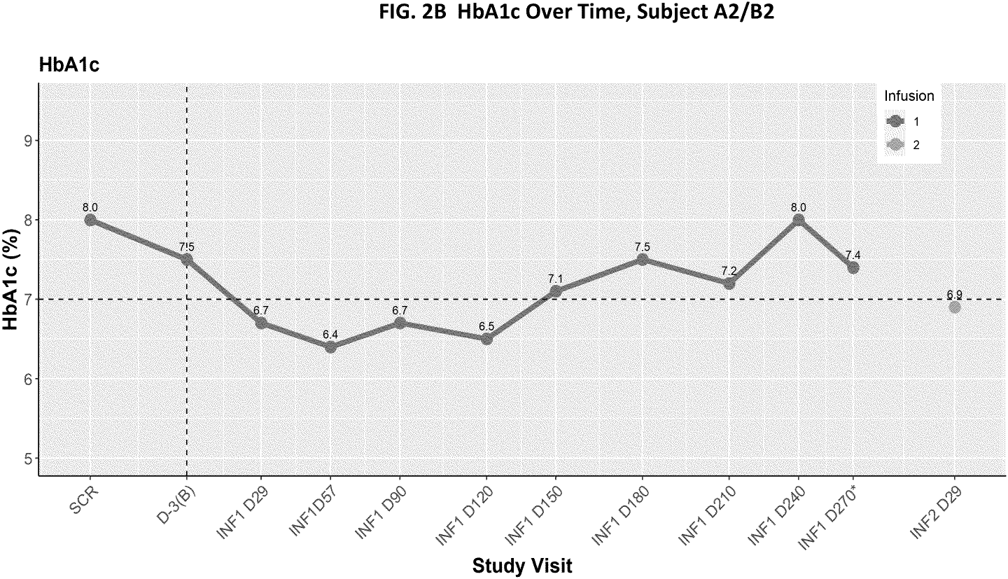

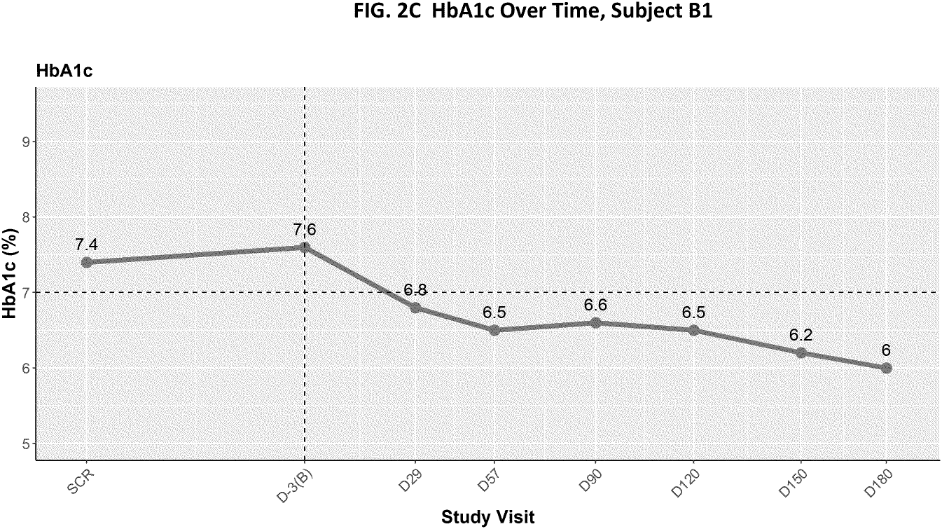

Subjects underwent frequent assessments HbA1c after treatment with SC-islets (FIG. 2A, FIG. 2B and FIG. 2C). Clinically meaningful reductions in HbA1c occurred in all subjects as early as Day 29, with all 3 subjects meeting the HbA1c target of <7% at certain time points. HbA1c <7% was maintained for Subjects A1 and B1 through the last assessment of at the data cutoff point of HbA1c (5.4% for Subject A1 at Month 15 and 6.0% for Subject B1 at Day 180). HbA1c for Subject A2/B2 increased to 8% by Day 240 after the first infusion, and then decreased to 6.9% at Day 29 after infusion of the second dose of SC-islets.

### Subject A1

Subject A1 met the HbA1c target of <7% (American Diabetes Association, 2020) by Day 150, and HbA1c continued to progressively decline over time to 5.2% at Day 270 (-3.4% change from baseline). HbA1c was stable after Day 270 and a decrease compared to baseline was sustained through the assessment at Month 15, when HbA1c was 5.4%(FIG. 2A and Table 4).

### Subject A2/B2

For Subject A2/B2, an HbA1c of 7.5% was observed at baseline, and clinically meaningful reduction in HbA1c to <7% was observed at Day 29 and sustained through Day 120 after the first infusion of SC-islets (FIG. 2B and Table 4). For Subject A2/B2 an HbA1c >7% was observed from Day 150 until the second infusion on Day 269.

### Subject B1

For Subject B1, HbA1c was 7.6% at baseline and progressively decreased after infusion with SC-islets. At Day 180, HbA1c was 6.0%, a -1.6% change from baseline (FIG. 2C and Table 4).

TABLE 4

Change from Baseline in HbA1c Over Time

| Change From Baseline at Study Visit | Subject A1 | Subject A2/B2 | Subject B1 |
|---|---|---|---|
| | HbA1c | HbA1c | HbA1c |
| Baseline | 8.6 | 7.5 | 7.6 |
| Day 29 | -0.9 | -0.8 | -0.8 |
| Day 57 | -0.7 | -1.1 | -1.1 |
| Day 90 | -1.4 | -0.8 | -1.0 |
| Day 120 | -1.6 | -1.0 | -1.1 |
| Day 150 | -1.9 | -0.4 | -1.5 |
| Day 180 | -1.7 | 0.0 | -1.6 |
| Day 210 | -- | -0.3 | |
| Day 240 | -- | 0.5 | |
| Day 270 | -3.4 | -- | |
| Day 300 | -3.4 | -- | |
| Day 330 | -3.2 | -- | |
| Day 365 | -3.4 | -- | |
| Month 15 | -3.2 | -- | -- |
| INF2 Day 29 | N/A | -0.6 | N/A |

HbA1c: hemoglobin A1c;
INF2: 2^{nd} infusion of SC-islets;
N/A: not applicable

### CGM-Derived Parameters

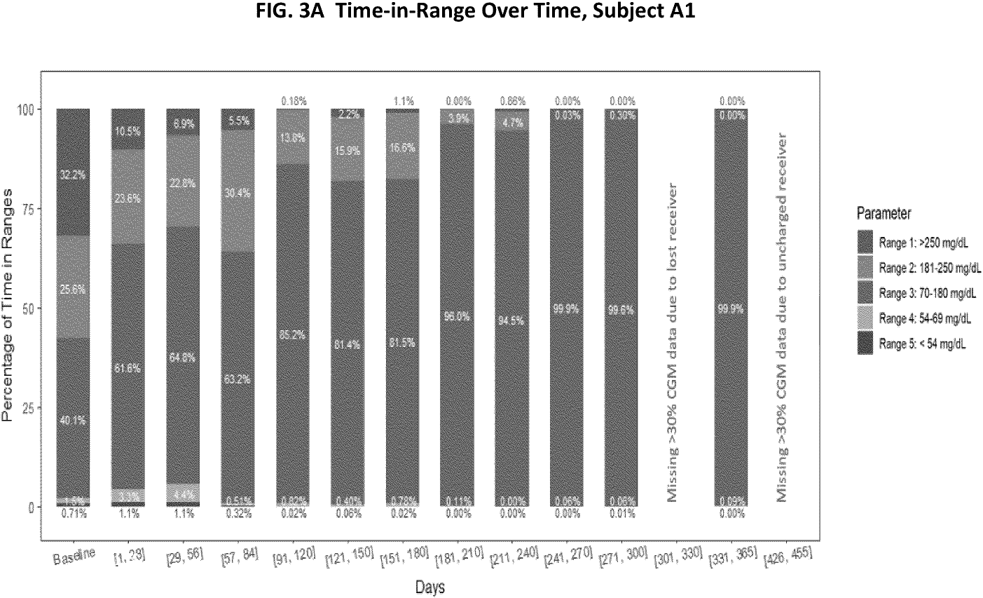

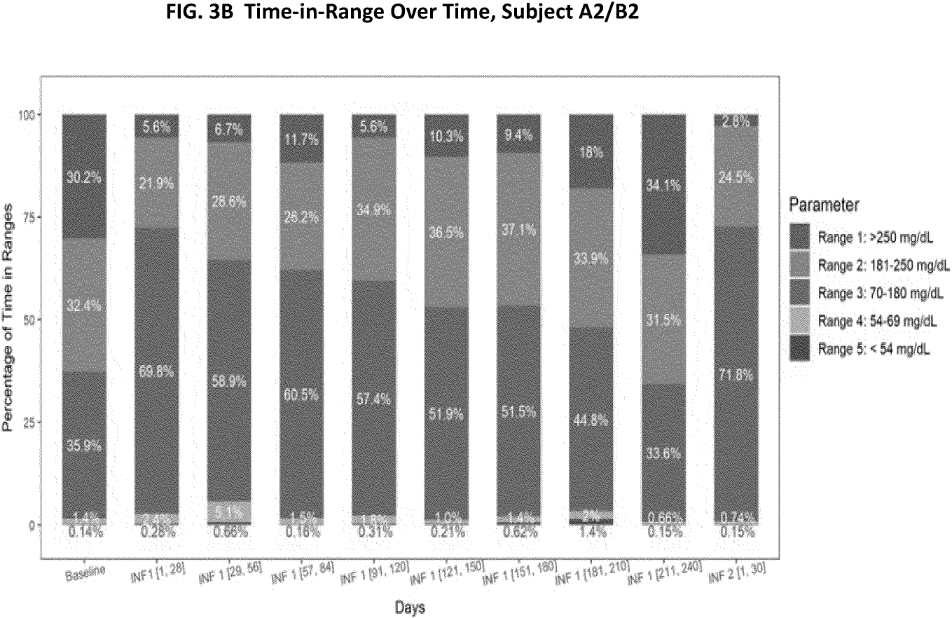

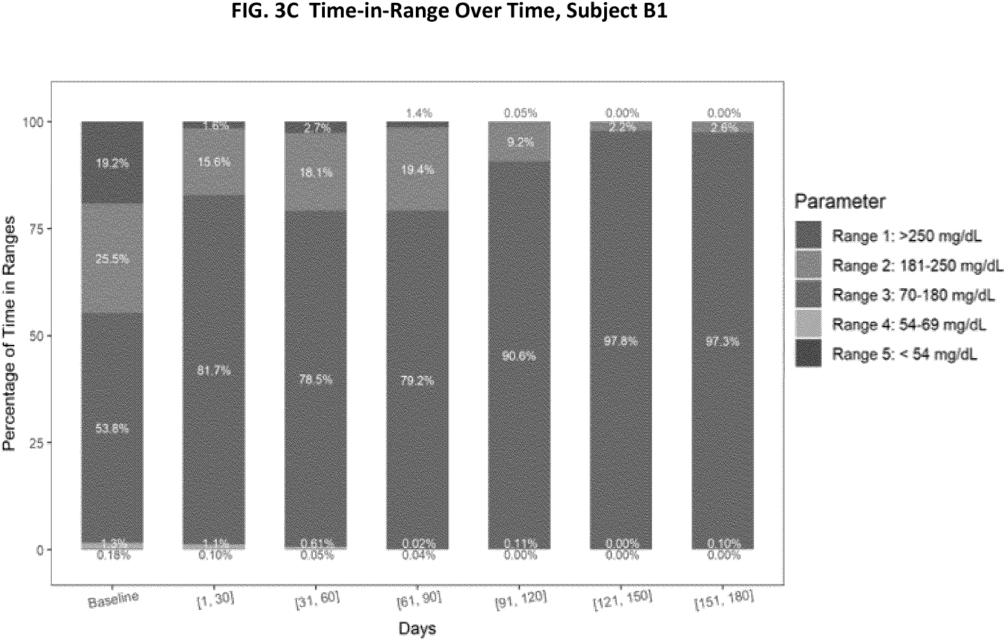

Subjects were required to continuously wear a sponsor-provided continuous glucose monitoring (CGM) device from approximately 4 weeks before SC-islet infusion (baseline) and for at least 365 days after infusion with SC-islets. Data for all subjects through the data cut-off date are summarized in FIG. 3A, FIG. 3B and FIG. 3C. Compared to baseline, there was an increase in time-in-range (70 to 180 mg/dL [3.9 to 10.0 mmol/L]) measured by CGM and a reduction in time-above-range (>180 mg/dL [>10.0 mmol/L)) for Subject A1 and Subject B1; for Subject A2, time-in-range increased at Day 29 after the first SC-islet infusion and remained above baseline through Day 210 after first infusion. For Subject A1, time-below-range (<70 mg/dL [<3.9 mmol/L]) decreased after Day 56; for Subject B1, time-below-range decreased after SC-islet infusion.

### Subject A1

Time-in-range progressively increased for Subject A1 (FIG. 3A). From Day 270 through Day 365, time-in-range for Subject A1 was >99%. A progressive and meaningful reduction of time <54 mg/dL (3.0 mmol/L) was also observed after Day 56 that correlated with the absence of SHEs after Day 35.

### Subject A2/B2

Time-in-range increased from 35.9% at baseline to 69.8% at Day 29 after the first infusion and remained above baseline through Day 210 after the first infusion (FIG. 3B). Time-above-range (>180 mg/dL [>10.0 mmol/L]) was reduced at Day 29 after the first infusion and a reduction compared to baseline was maintained through Day 210 after the first infusion.

### Subject B1

Time-in-range increased from 53.8% at baseline to 97.3% at Day 180, with concomitant reductions in time-above-range and time-below-range (FIG. 3C.).

### Change in Exogenous Insulin Total Daily Dose

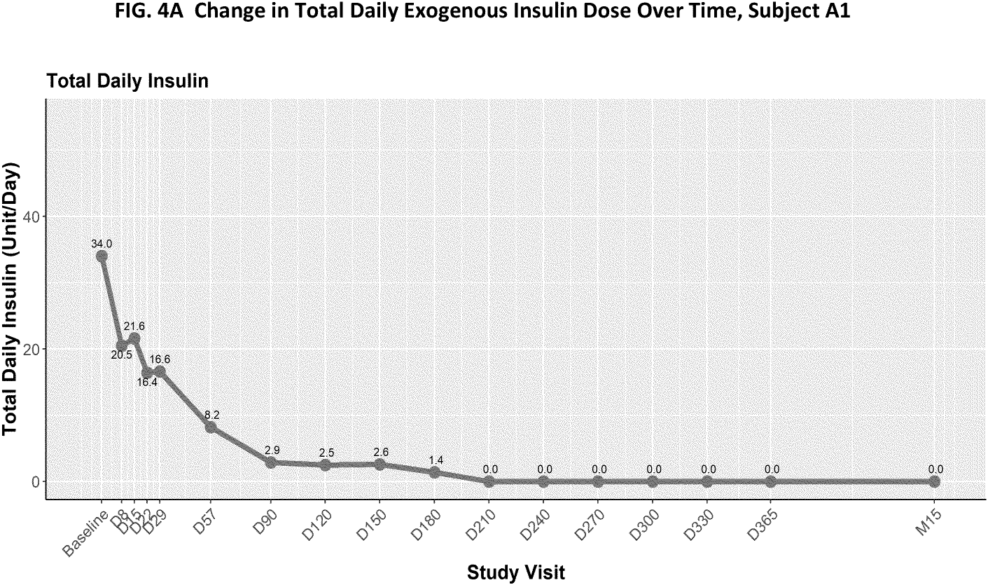

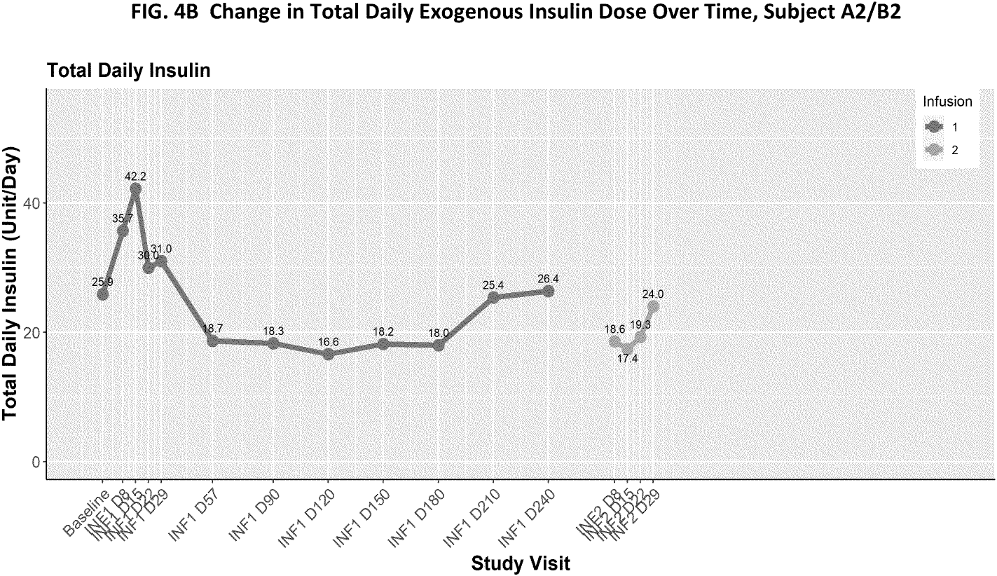

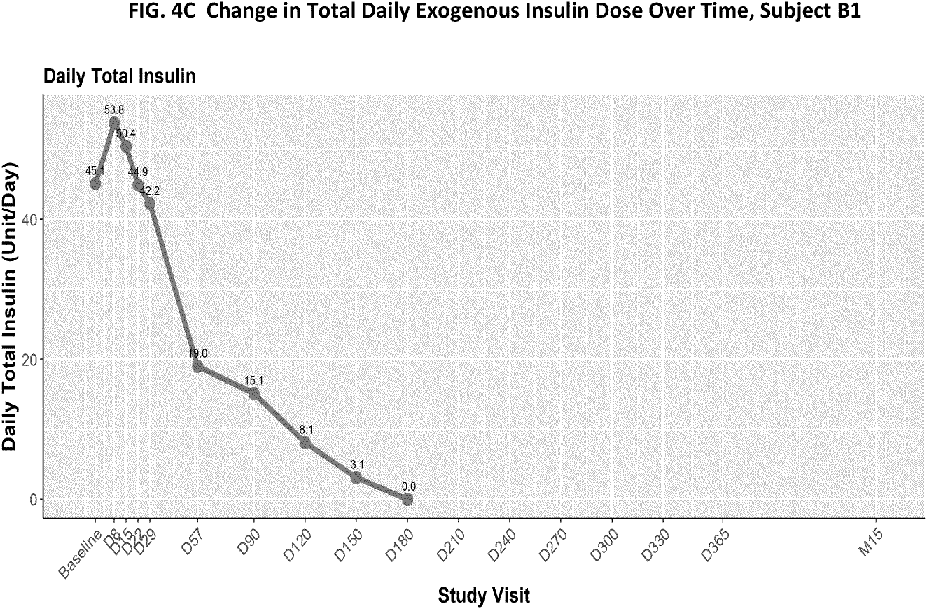

Assessment of exogenous insulin dose was performed regularly for all subjects beginning at baseline as shown in FIG. 4A, FIG. 4B and FIG. 4C. In all 3 subjects there was a clinically significant reduction in exogenous daily insulin dose by Day 90 after SC-islet treatment. In Subject A1 and Subject B1, exogenous daily insulin dose declined to zero, with improved glycemic control, and both subjects were deemed to meet the definition for insulin independence.

### Subject A1

Total daily exogenous insulin dose (mean value over 7 consecutive days [when available]) was 34 U/day at baseline decreased with time after dosing with SC-islets. Beginning at Day 210, and sustained through Month 15, Subject A1 had a mean total daily insulin dose of 0 U/day (100% reduction from baseline; FIG. 4A).

### Subject A2/B2

Total daily insulin dose for Subject A2/B2 was 25.9 U/day at baseline, and progressively decreased until Day 120 (-35.9% change from baseline) after the first SC-islet infusion. At subsequent follow-up visits through Day 240 after the first infusion, total daily insulin dose for Subject A2/B2 increased and it was similar to the baseline value at Day 240 (FIG. 4B).

### Subject B1

Total daily insulin dose for Subject B1 was 45.1 U/day at baseline and decreased to 0.0 U/day at Day 180 (100% reduction from baseline; FIG. 4C).

This study surprisingly demonstrates that the SC-islet cells disclosed herein are efficacious at a lower dose of cells than that suggested in the art. For example, in a separate study by Ramzy et al. (2021, Cell Stem Cell, 28, 2047-2061), subjects administered up to 5 × 10⁸ of stem cell-derived pancreatic endoderm cells (“PECs”) failed to show a max peak stimulated C-peptide greater than around 40 pM even after 26 or 52 months post-implantation, whereas Subjects A1 and A2 treated with 4.0 × 10⁸ SC-islet cells showed a max peak stimulated C-peptide of 560 pM and 202 pM, respectively, after only 90 days. In addition, while the treated subjects in Ramzy et al. were on average stable for HbA1c, Subject A1 showed a 3.9% HbA1c reduction at 270 days, while Subject A2 showed a 0.6% HbA1c reduction at 270 days. Moreover, while the treated subjects in Ramzy et al. showed only an average of 20% reduced insulin requirements, Subject A1 showed a 100% reduced insulin requirement by day 210, and Subject B2 (who received a single dose of 8.0 × 10⁸ SC-islet cells) was insulin independent by day 180. Ramzy et al. postulates that implanting more of its PECs could enhance outcomes, while the present study surprisingly demonstrates that SC-islets as described herein are efficacious at doses as low as 4.0 × 10⁸ SC-islet cells.

The present study also suggests that fewer SC-islet cells are needed for efficacy as compared to cadaveric islet-based therapies. Human cadaveric islets are measured in “islet equivalents” or “IEQs” based upon a 150 mm diameter islet. Based on the number of β-cells per IEQ and based on the percentage of β-cells in the clusters (“IEQs”) in the compositions administered to Subjects A1, A2/B2, and B1, the doses of 0.4 × 10⁹ and 0.8 × 10⁹ SC-islet cells may be similar to approximately 4300 IEQ/kg and 8600 IEQ/kg, respectively, for a 70 kg individual. Previous cadaveric islet studies suggest using doses higher than 4300 or 8600 IEQ/kg doses for treating diabetic patients. For example, Hering et al. (2016, Diabetes Care, 39:1230-40) referred to patients receiving a median of 11,972 IEQ/kg (with a range of 5,227-25,553 IEQ/kg) of cadaveric islet cells. Similarly, Ramzy et al. (citing Shapiro et al. 2000, N. Engl. J. Med. 343, 230-238) suggests that islet transplant recipients have better outcomes with a total islet dose of >11,000 IEQ/kg body weight. The present SC-islet study surprisingly shows strong clinical efficacy using cell doses that are well lower than 11,000 IEQ/kg.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure can be employed in practicing the present disclosure. It is intended that the following claims define the scope of the present disclosure and that methods and structures within the scope of these claims and their equivalents be covered thereby.

What is claimed is:

1. A method of treating a subject in need thereof, comprising: administering to the subject via infusion a first pharmaceutical composition comprising a population of cells in a liquid suspension, wherein the population of cells comprises from 1 × 10⁸ to 10 × 10⁸ from 3 × 10⁸ to 8.5 × 10⁸, from 4 × 10⁸ to 8.5 × 10⁸, or from 5 × 10⁸ to 8.5 × 10⁸ cells, and wherein the population of cells comprises non-native cells expressing C-peptide and ISL1.

2-16. (canceled)

17. The method of claim 1, wherein prior to the administration, the subject has a baseline Hb1Ac level of more than 7.0%, 7.3%, 7.8%, 8.0%, 8.1%, 8.2%, 8.3%, 8.4%, 8.5%, 8.6%, 8.8%, or 9.0%.

18. The method of claim 1, wherein prior to the administration, the subject receives infusion of insulin at a level of at least about 20 U/day, 22 U/day, 24 U/day, 26 U/day, 28 U/day, 30 U/day, 32 U/day, or 34 U/day.

19. The method of claim 1, wherein prior to the administration, the subject has a medical history of severe hypoglycemic events.

20. The method of claim 1, wherein prior to the administration, the subject has a medical history of impaired hypoglycemia awareness.

21. The method of claim 1, wherein prior to the administration, the subject presented with no residual endogenous islet cell function.

22. (canceled)

23. The method of claim 21, wherein the no residual endogenous islet cell function is indicated by sustained stimulated glucose levels greater than 350, 400, 450, 475, or 500 mg/dL at the Mixed Meal Tolerance Test.

24-27. (canceled)

28. The method of claim 1, wherein the subject has Type 1 diabetes.

29-30. (canceled)

31. The method of claim 1, wherein the population of cells comprises about about 3.5 × 10⁸ to about 4.5 × 10⁸ cells.

32. The method of claim 1, wherein the population of cells comprises about about 3.5 × 10⁸ to about 8.5 × 10⁸ cells.

33. The method of claim 1, wherein the subject is administered via infusion a second pharmaceutical composition comprising a population of cells in a liquid suspension, wherein the population of cells comprises from 1 × 10⁸ to 10 × 10⁸ from 3 × 10⁸ to 8.5 × 10⁸, from 4 × 10⁸ to 8.5 × 10⁸, or from 5 × 10⁸ to 8.5 × 10⁸ cells, and wherein the population of cells comprises non-native cells expressing C-peptide and ISL1, wherein the second pharmaceutical composition is administered to the subject at a later point in time than the first pharmaceutical composition.

34. The method of claim 33, wherein the second pharmaceutical composition is administered to the subject at least 3 months, 4 months, 5 months, 6 months, 7 months, 8 months, 9 months, 10 months, 11 months, 12 months, 15 months or between 3-12 months, 3-10 months, 3-9 months, 3-7 months, 3-5 months, 5-12 months, 8-10 months, 7-12 months, 9-15 months, or 9-12 months after the subject is administered the first pharmaceutical composition.

35. The method of claim 33, wherein the first pharmaceutical composition comprises 3.5 × 10⁸ to about 8.5 × 10⁸ cells and wherein the second pharmaceutical composition comprises 3.5 × 10⁸ to about 8.5 × 10⁸ cells.

36. (canceled)

37. The method of claim 1, wherein the subject is also administered at least one immunosuppressant.

38. The method of claim 37, wherein the at least one immunosuppressant comprises selected from the group consisting of Thymoglobulin, Etanercept, Basiliximab, Tacrolimus, Sirolimus, and Mycophenolate mofetil.

39-43. (canceled)

44. The method of claim 1, wherein the first pharmaceutical composition comprises a sugar.

45. (canceled)

46. The method of claim 44, wherein the liquid suspension comprises the sugar at a concentration of between about 0.05% and about 1.5%.

47. The method of claim 1, wherein the first pharmaceutical composition comprises a CMRL medium.

48. The method of claim 1, wherein the first pharmaceutical composition comprises HypoThermosol® FRS Preservation Media.

49. (canceled)

50. The method of claim 1, wherein:
(a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells in the first pharmaceutical composition express C-peptide and ISL1 but not VMAT1;
(b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells in the first pharmaceutical composition express glucagon but not somatostatin; and/or
(c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells in the first pharmaceutical composition express somatostatin but not glucagon.

51. The method of claim 1, wherein:
(a) 30-90%, 30-80%, 30-70%, 30-60%, 30-50%, 30-40%, 40-90%, 40-80%, 40-70%, 40-60%, 40-50%, 50-90%, 50-80%, 50-70%, 50-60%, 60-90%, 60-80%, 60-70%, 70-90%, 70-80%, 70-90%, 70-80%, or 80-90% of the cells in the population of cells express C-peptide and ISL1 but not VMAT1;
(b) 3-40%, 3-35%, 3-30%, 3-25%, 3-20%, 3-15%, 3-10%, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, 30-35% or 35-40% of the cells in the population of cells express glucagon but not somatostatin; and
(c) 1-20%, 1-15%, 1-12%, 1-10%, 1-8%, 1-5%, 2-20%, 2-15%, 2-12%, 2-10%, 2-8%, 2-5%, 3-20%, 3-15%, 3-12%, 3-10%, 3-8%, 3-5%, 4-20%, 4-15%, 4-12%, 4-10%, 4-8%, 4-5%, 5-20%, 5-15%, 5-12%, 5-10%, 5-8%, 7-20%, 7-15%, 7-12%, 7-10%, 9-20%, 9-15%, 9-12%, 8-10%, 8-12%, 8-15%, 8-20%, 10-20%, 10-12%, 10-15%, 12-20%, 12-15% or 15-20% of the cells in the population of cells express somatostatin but not glucagon.

52. The method of claim 1, wherein:
(a) 35-60% of the cells in the population of cells express C-peptide and ISL1 but not VMAT1;
(b) 4-25%, of the cells in the population of cells express glucagon but not somatostatin; and
(c) 1-10% of the cells in the population of cells express somatostatin but not glucagon.

53. The method of claim 1, wherein:
(a) 40-60% of the cells in the population of cells express C-peptide and ISL1 but not VMAT1;
(b) 10-25%, of the cells in the population of cells express glucagon but not somatostatin; and
(c) 4-10% of the cells in the population of cells express somatostatin but not glucagon.

54-61. (canceled)

62. The method of claim 1, wherein between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, 40-50% of the cells in the pharmaceutical composition are NKX6.1⁺/ISL1⁺ cells, as determined by flow cytometry.

63. The method of claim 1, wherein between 20-60%, 20-50%, 20-45%, 20-40%, 20-35%, 20-30%, 20-25%, 25-50%, 25-40%, 25-35%, 30-60%, 30-50%, 30-40%, 30-35%, 35-50%, or 40-50% of the cells in the pharmaceutical composition are NKX6.1⁻/ISL1⁺ cells, as determined by flow cytometry.

64. The method of claim 1, wherein between 20-50%, 20-45%, 20-40%, 20-35%, 20-30%, 20-25%, 25-50%, 25-40%, 25-35%, 30-60%, 30-50%, 30-40%, 30-35%, 35-50%, 40-50%, 10-20%, or 10-25% of the cells in the pharmaceutical composition are NKX6.1⁺/ISL1⁻ cells, as determined by flow cytometry.

65. The method of claim 1, wherein:
a) at least 30% of the cells in the composition are NKX6.1-positive, ISL1-positive cells;
b) at least 25% of the cells in the composition are NKX6.1-negative, ISL1-positive cells;
c) less than 12% of the cells in the composition are NKX6.1-negative, ISL1-negative cells; and/or
d) between 9-25% of the cells in the composition are NKX6.1-positive, ISL1-negative cells.

66. (canceled)

67. The method of claim 1, wherein the composition comprises NKX6.1⁺/ISL1⁺ cells that display a GSIS in vivo.

68-71. (canceled)

72. The method of claim 1, wherein at least a portion of the cells in the population of cells are present in plurality of cell clusters.

73. The method of claim 72, wherein the cell clusters are about 50 µm to about 500 µm, about 50 µm to about 400 µm, about 50 µm to about 300 µm, about 60 µm to about 400 µm, about 60 µm to about 300 µm, about 60 µm to about 250 µm, about 75 µm to about 400 µm, about 75 µm to about 300 µm, about 75 µm to about 250 µm, about 125 µm to about 225 µm, about 130 µm to about 160 µm, about 170 µm to about 225 µm, about 140 µm to about 200 µm, about 140 µm to about 170 µm, about 160 µm to about 220 µm, about 170 µm to about 215 µm, or about 170 µm to about 200 µm in diameter.

74-82. (canceled)

83. The method of claim 1, wherein the first pharmaceutical composition is infused into portal vein of the subject.

84. The method of claim 1, wherein the subject receives a single dose of the first pharmaceutical composition.

85-100. (canceled)

101. A pharmaceutical composition formulated for infusion, comprising a population of cells in a liquid suspension, wherein the population of cells comprises from about 1 × 10⁸ to about 10 × 10⁸, from 3 × 10⁸ to 8.5 × 10⁸, from 4 × 10⁸ to 8.5 × 10⁸, or from 5 × 10⁸ to 8.5 × 10⁸ cells, and wherein the population of cells comprises non-native cells expressing C-peptide and ISL1.

102-126. (canceled)

127. A kit, comprising:
(a) the pharmaceutical composition of claim 101, and
(b) instruction for administering the pharmaceutical composition to a subject in need thereof.

128. A kit, comprising:
(a) a pharmaceutical composition formulated for infusion, wherein the pharmaceutical composition comprises a population of cells in suspension, wherein the population of cells comprises at least about 1 × 10⁸ cells, and wherein the population of cells comprises non-native cells expressing C-peptide and ISL1; and
(b) instruction for administering the pharmaceutical composition to a subject in need thereof.

129-143. (canceled)

144. An encapsulation composition comprising between 40 × 10⁶ cells and 100 × 10⁶ cells, between 50 × 10⁶ cells and 90 × 10⁶ cells, between 60 × 10⁶ cells and 80 × 10⁶ cells, or between 70 × 10⁶ cells and 80 × 10⁶ SC-islet cells.

145-156. (canceled)

157. A method of treating a subject having Type I diabetes, comprising administering to the subject a device of claim 144.

158-169. (canceled)


SEQUENCE LISTING (17 sequences)

SEQ ID NO: 1 - protein, 116 aa, synthetic construct [sequence omitted]

SEQ ID NO: 2 - protein, 426 aa, synthetic construct
  REGION 1..426: note = polypeptide (TGF-Beta)
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 3 - protein, 130 aa, synthetic construct
  REGION 1..130: note = polypeptide (TGF-Beta)
ARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 4 - protein, 115 aa, synthetic construct
  REGION 1..115: note = polypeptide (TGF-Beta)
GLECDGKVNICCKKQFFVSFKDIGWNDWIIAPSGYHANYCEGECPSHIAGTSGSSLSFHSTVINHYACGHSPFANLKSCCVPTKLRPMSMLYYDDGQNIIKKDIQNMIVEECGCS

SEQ ID NO: 5 - protein, 426 aa, synthetic construct; identical to SEQ ID NO: 2
  REGION 1..426: note = polypeptide (TGF-Beta)

SEQ ID NO: 6 - protein, 424 aa, synthetic construct
  REGION 1..424: note = polypeptide (TGF-Beta)
MPLLWLRGFLLASCWIIVRSSPTPGSEGHGSAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 7 - protein, 424 aa, synthetic construct
  REGION 1..424: note = polypeptide (TGF-Beta)
MPLLWLRGFLLASCWIIVRSSPTPGSEGHGAAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDMGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 8 - protein, 424 aa, synthetic construct
  REGION 1..424: note = polypeptide (TGF-Beta)
MPLLWKRGFLLVICWIIVRSSPTPGSEGHSSVADCPSCALTTLSKDVPSSQPEMVEAVKKHILNMLHLRDRPNITQPVPKAALLNATKKLHVGKVGDDGYVEIEDDVGRRAEMNEVVEQTSEIITFAESGTPKKTLHFEISKEGSELSVVEHAEVWLFLKVSKANRSRTKVTIRLFQQQRQPKGNSEAAEDMEDMGLKGERSETLISEKAVDARKSTWHIFPISSSVQRLLDQGQSSLDVRIACDLCQETGASLVLLGKKKKKEDDGEGKEKDGGELTGEEEKEQSHRPFLMMLARHSEDRQHRRRERGLECDGKVNICCKKQFFVSFKDIGWSDWIIAPTGYHANYCEEECPSHIAGTSGSSLSFHSTVINHYRMRGHSPFANLKSCCVPTKLRPMSMLYYDDGQNIIKKDIQNMIVEECGCS

SEQ ID NO: 9 - protein, 425 aa, synthetic construct
  REGION 1..425: note = polypeptide (TGF-Beta)
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKKKEEEGEGKKRDGEGGAGGDEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 10 - protein, 426 aa, synthetic construct
  REGION 1..426: note = polypeptide (TGF-Beta)
MPLLWLRGFLLASCWIIVKSSPTPGSEGHSAAPNCPSCALATLPKDVPNAQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRSKVTIRLLQQQKHPQGSSDTREEAEEADLMEERSEQLISEKVVDARKSTWHIFPVSSSIQRLLDQGKSSLDIRIACDQCHETGASLVLLGKKKKKEEEGEGKKKDGGEAGAGVDEEKEQSHRPFLMLQARQSEDHPHRRRRRGLECDGKVNICCKKQFFVSFKDIGWNDWIIAPSGYHANYCEGECPSHIAGTSGSSLSFHSTVINQYRLRGHNPFANLKSCCVPTKLRPMSMLYYDDGQNIIKKDIQNMIVEECGCS

SEQ ID NO: 11 - protein, 424 aa, synthetic construct
  REGION 1..424: note = polypeptide (TGF-Beta)
MPLLWLRGFLLASCWIIVRSSPTPGSGGHSAAPDCPSCALATLPKDVPNSQPEMVEAVKKHILNMLHLKKRPDVTQPVPKAALLNAIRKLHVGKVGENGYVELEDDIGRRAEMNELMEQTSEIITFAEAGTARKTLRFEISKEGSDLSVVERAEIWLFLKVPKANRTRTKVSIRLFQQQRRPQGSADAGEEAEDVGFPEEKSEVLISEKVVDARKSTWHIFPVSSSIQRLLDQGKSALDIRTACEQCHETGASLVLLGKKKKKEEEAEGRKRDGEGAGVDEEKEQSHRPFLMLQARQSEEHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 12 - protein, 425 aa, synthetic construct
  REGION 1..425: note = polypeptide (TGF-Beta)
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISQEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKRKEEEGEGKKRDGEGGAGGDEEKEQSHRPFLMLQARQSEDHPHRRRRRGLECDGKVNICCKKQFYVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 13 - protein, 424 aa, synthetic construct
  REGION 1..424: note = polypeptide (TGF-Beta)
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 14 - protein, 395 aa, synthetic construct
  REGION 1..395: note = polypeptide (TGF-Beta)
MSPLPLLSGILLLLIRSCSLSAMVTKGSLPMSEQQAGATVCPSCALARFRKGVSESEDEGAQQDVVEAVKRHILNMLHLQERPNITHPVPRAALLNAIRKVHVGRVAKDGSVLIEDEASNRAETEQAEQTEIITFAETGEAPGIVNFLISKEGGEMSVVDQANVWIFLRLPKGNRTRANVNIRLLLQQGAGEKILAEKSVDTRRSGWHTFPASESVQSLLQRGGSTLSLRVSCPLCADARATPVLVSPGGSEREQSHRPFLMAVVRQMDELSLRRRRKRGLECDGKARVCCKRQFYVNFKDIGWNDWIIAPSGYHANYCEGDCASNVASITGNSLSFHSTVISHYRIRGYSPFTNIKSCCVPTRLRAMSMLYYNEEQKIVKKDIQNMIVEECGCS

SEQ ID NO: 15 - protein, 404 aa, synthetic construct
  REGION 1..404: note = polypeptide (TGF-Beta)
MSSLTLVNRGTAALRLFVRGLLTHSSREWLSGDGEPDDPVTPCPSCALAQRQKDSEEQTDMVEAVKRHILNMLHLNTRPNVTHPVPRAALLNAIRRLHVGRVGEDGTVEMEEDGGGLGEHREQSEEQPFEIITFAEPGDAPDIMKFDISMEGNTLSVVEQANVWLLLKVAKGSRGKGKVSVQLLQHGKADPGSADGPQEAVVSEKTVDTRRSGWHTLPVSRTVQTLLDGDSSMLSLRVSCPMCAEAGAVPILVPTESNKGKEREQSHRPFLMVVLKPAEEHPHRRSKRGLECDGKIRVCCKRQFYVNFKDIGWSDWIIAPSGYHANYCEGDCPSHVASITGSALSFHSTVINHYRMRGYSPFNNIKSCCVPTRLRAMSMLYYNEEQKIIKKDIQNMIVEECGCS

SEQ ID NO: 16 - protein, 194 aa, synthetic construct
  REGION 1..194: note = polypeptide (FGF)
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 17 - protein, 176 aa, synthetic construct
  REGION 1..176: note = polypeptide (EGF)
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETN